(19) United States
(12) Patent Application Publication
(10) Pub. No.: US 2024/0318120 A1
(43) Pub. Date: Sep. 26, 2024

(54) APPARATUSES, METHODS, AND KITS FOR MICROFLUIDIC ASSAYS

(71) Applicant: BRUKER CELLULAR ANALYSIS, INC., Emeryville, CA (US)

(72) Inventors: Patrick N. INGRAM, Emeryville, CA (US); Alexander CHIEN, Berkeley, CA (US); Ke-Chih LIN, Richmond, CA (US); Or GADISH, Richmond, CA (US); Troy A. LIONBERGER, San Francosco, CA (US); Eric K. SACKMANN, Berkeley, CA (US); Volker L.S. KURZ, Oakland, CA (US); Alexander J. MASTROIANNI, Alameda, CA (US); Randall D. LOWE, JR., Emeryville, CA (US); Jonathan Cloud Dragon HUBBARD, Oakland, CA (US)

(73) Assignee: BRUKER CELLULAR ANALYSIS, INC., Emeryville, CA (US)

(21) Appl. No.: 18/043,262

(22) PCT Filed: Aug. 30, 2021
(86) PCT No.: PCT/US2021/048196

§ 371 Date: Feb. 27, 2023

Related U.S. Application Data
(60) Provisional application No. 63/072,849, filed on Aug. 31, 2020.
(60) Provisional application No. 63/235,660, filed on Aug. 20, 2021.

(51) Int. Cl. C12M 1/34; C12M 1/00; C12M 3/06; G01N 21/05; G01N 21/64; G01N 21/77

(52) U.S. Cl. CPC C12M 41/34; C12M 23/16; C12M 41/06; C12M 41/36; G01N 21/05; G01N 21/6408; G01N 21/6458; G01N 2021/6432; G01N 2021/6482; G01N 2021/7786

(57) ABSTRACT

Methods, systems, and kits for determining a level of dissolved oxygen within a microfluidic device are provided. The microfluidic device can be suitable for cell culture. The methods, systems, and kits can further be used to determine a level of oxygen consumption in a population of biological micro-objects. In particular, the methods, systems, and kits of the present disclosure rely on flowing a fluidic medium containing a dye and a supplied partial pressure of oxygen into the microfluidic device for a period of time, wherein fluorescence emitted by the dye changes based on availability of oxygen proximate to the dye; taking a fluorescence image of an area of interest within the chamber; and correlating fluorescence of the fluorescence image of the area of interest to a reference to determine an observed partial pressure of the oxygen in the area of interest.

### CROSS-REFERENCE

The present application claims priority to U.S. Provisional Application No. 63/072,849, filed on Aug. 31, 2020, entitled “METHODS, SYSTEM AND KITS FOR IN-PEN ASSAYS,” and U.S. Provisional Application No. 63/235,660, filed on Aug. 20, 2021, entitled “APPARATUSES, METHODS, AND KITS FOR MICROFLUIDIC ASSAYS,” each of which applications is entirely incorporated herein by reference for all purposes.

### BACKGROUND

Oxygen consumption levels may be correlated with the health, viability, and/or productivity of a population of cells. Thus, it may be critical to measure oxygen levels within culture systems and/or monitor cellular oxygen consumption in order to assess such parameters. However, current microfluidic cell culture systems generally do not provide the ability to measure oxygen levels or monitor oxygen consumption. Therefore, there is a need for systems and methods for measuring oxygen levels, including methods that allow for measuring oxygen levels at multiple locations or even throughout such microfluidic cell culture systems. There is also a need for systems and methods for monitoring oxygen consumption by cells being cultured in such microfluidic devices. Further, most microfluidic devices are constructed using materials permeable to oxygen, which may reduce the accuracy of the oxygen consumption level measurements. Thus, there is a need for microfluidic devices that are substantially impermeable to oxygen.

### SUMMARY

In one aspect, a method of determining a level of oxygen in a medium disposed within a microfluidic device comprising a flow region and one or more chambers fluidically coupled to the flowing region is provided. In accordance with various embodiments, the method comprises: flowing a fluidic medium containing a dye and a supplied partial pressure of oxygen into the microfluidic device for a period of time, wherein fluorescence emitted by the dye changes based on availability of oxygen proximate to the dye; taking a fluorescence image of an area of interest (AOI) within the flow region or one or more of the chambers; and correlating fluorescence detected in the fluorescence image of the AOI with a reference to determine an observed level (e.g., a partial pressure) of oxygen in the AOL. In accordance with various embodiments, the method further comprises: determining a level of oxygen consumption by a biological micro-object or a population of biological micro-objects (e.g., a clonal population) disposed within one of the one or more chambers. In accordance with various embodiments, the method further comprises: comparing the determined level of oxygen consumption with a threshold value; and selecting the biological micro-object or the population of biological micro-objects (e.g., a clonal population) if the determined level of oxygen consumption is above the threshold value. In accordance with various embodiments, the method further comprises: forecasting a level of productivity of an expanded population of biological micro-objects expanded from the biological micro-object or the population of biological micro-objects based at least in part upon the determined level of oxygen consumption. In accordance with various embodiments, the method further comprises: determining a number of biological micro-objects present in the chamber, wherein the forecast level of productivity is based at least in part on the determined number of biological micro-objects in the chamber. In accordance with various embodiments, the method further comprises: comparing the forecast level of productivity with a threshold value; and selecting the biological micro-object or the population of biological micro-objects (e.g., a clonal population) if the forecast level of productivity is above the threshold value. In accordance with various embodiments, the selected biological micro-object or the population of biological micro-objects is removed from the microfluidic device (e.g., exported) and, optionally, cultured so as to produce an expanded population of biological micro-objects. In accordance with various embodiments, the expanded population of biological micro-objects is expanded at least partially following export from the microfluidic device (e.g., in a macro-scale culture device).

In another aspect, a method of determining a level of oxygen consumption by a biological micro-object or a population of biological micro-objects is provided. In accordance with various embodiments, the method comprises: disposing the biological micro-object or the clonal population of biological micro-objects in a chamber of a microfluidic device comprising a flow region, wherein the chamber is fluidically connected to the flow region; flowing a fluidic medium containing a dye and a supplied partial pressure of oxygen into the microfluidic device for a period of time, wherein fluorescence emitted by the dye changes based on availability of oxygen proximate to the dye; taking a fluorescence image of an area of interest within the flow region and/or the chamber; correlating fluorescence detected in the fluorescence image of the area of interest with a reference to determine an observed level (e.g., partial pressure) of oxygen in the area of interest; and determining the level of oxygen consumption by the biological micro-object or the population of biological micro-objects disposed within the chamber.

In another aspect, a method of selecting a biological micro-object or a population of biological micro-objects is provided. In accordance with various embodiments, the method comprises: disposing the biological micro-object or the clonal population of biological micro-objects in a chamber of a microfluidic device comprising a flow region, wherein the chamber is fluidically connected to the flow region; flowing a fluidic medium containing a dye and a supplied partial pressure of oxygen into the microfluidic device for a period of time, wherein fluorescence emitted by the dye changes based on availability of oxygen proximate to the dye; taking a fluorescence image of an area of interest within the flow region and/or the chamber; correlating fluorescence detected in the fluorescence image of the area of interest with a reference to determine an observed level (e.g., partial pressure) of oxygen in the area of interest; determining the level of oxygen consumption by the biological micro-object or the population of biological micro-objects disposed within the chamber; and selecting the biological micro-object or the population of biological micro-objects if the determined level of oxygen consumption is above a threshold value.

In another aspect, a method of forecasting a level of productivity of a population of biological micro-objects expanded from a biological micro-object or a clonal population of biological micro-objects is provided. In accordance with various embodiments, the method comprises: disposing a biological micro-object or a clonal population of biological micro-objects in a chamber of a microfluidic device comprising a flow region, wherein the chamber is fluidically connected to the flow region; flowing a fluidic medium containing a dye and a supplied partial pressure of oxygen into the microfluidic device for a period of time, wherein fluorescence emitted by the dye changes based on availability of oxygen proximate to the dye; taking a fluorescence image of an area of interest within the flow region and/or the chamber; correlating fluorescence detected in the fluorescence image of the area of interest with a reference to determine an observed level (e.g., partial pressure) of oxygen in the area of interest; determining a level of oxygen consumption by the biological micro-object or the population of biological micro-objects disposed within the chamber; and forecasting a level of productivity of the expanded population of biological micro-objects expanded from the biological micro-object or the clonal population of biological micro-objects, wherein the forecast level of productivity is based at least in part upon the determined level of oxygen consumption. In accordance with various embodiments, the microfluidic device comprises a plurality of chambers, each fluidically connected to the flow region, wherein there is a plurality of biological micro-objects and/or populations of biological micro-objects, each one of the biological micro-objects and/or populations of biological micro-objects disposed within a corresponding chamber of the plurality of chambers, and wherein selecting the biological micro-object or the population of biological micro-objects comprises selecting one or more of the plurality of biological micro-objects and/or populations of biological micro-objects.

With regard to any of the foregoing aspects and embodiments, and in accordance with various embodiments thereof, the dye comprises a soluble and diffusible dye. In accordance with various embodiments, the dye comprises a ruthenium complex. In accordance with various embodiments, the fluorescence emitted by the dye is quenched when the dye is in proximity to oxygen and fluoresces when the dye is not in proximity to oxygen. In accordance with various embodiments, the level of oxygen corresponds to a decrease in the observed partial pressure of the oxygen compared to the supplied partial pressure. In accordance with various embodiments, the fluidic medium is flowed at a flow rate ranging between 0.1 microliter/s and 10 microliters/s. In accordance with various embodiments, the biological micro-object or population of biological micro-objects in the chamber of the microfluidic device is cultured under aerobic conditions comprising flowing a fluidic medium through the fluidic region, wherein the fluidic medium comprises at least a minimum supplied partial pressure of oxygen of 0.04 bar. In accordance with various embodiments, the AOI is disposed within the chamber at a location wherein transference of components of the fluidic medium flowing in the flow region (e.g., a channel to which the chamber is fluidically connected) is dominated by diffusion. In accordance with various embodiments, the AOI is disposed in the flow region (e.g., a channel), at a position proximal to an opening from the chamber to the flow region. In accordance with various embodiments, the AOI contains no biological micro-objects. In accordance with various embodiments, the fluidic medium comprises a liquid medium, a gaseous medium, or a mixture thereof. In accordance with various embodiments, the flowing the fluidic medium containing the dye and the supplied partial pressure of oxygen into the microfluidic device comprises alternately flowing a liquid medium into the microfluidic device and flowing a gaseous medium comprising the supplied partial pressure of oxygen into the microfluidic device. In accordance with various embodiments, the medium comprises a liquid medium saturated with the supplied partial pressure of the oxygen. In accordance with various embodiments, the correlating the fluorescence of the fluorescence image of the AOI to a reference to determine the observed partial pressure of the oxygen at the AOI comprises identifying an intensity of the fluorescence on a reference curve correlating fluorescence intensities of the dye with supplied partial pressures of the oxygen. In accordance with various embodiments, the methods further comprise constructing a reference curve correlating an observed intensity of fluorescence of the dye with a supplied partial pressure of oxygen. In accordance with various embodiments, the constructing the reference curve comprises: flowing a liquid medium through the channel of the microfluidic device for a first reference period of time, wherein the liquid medium comprises the dye and a first supplied partial pressure of the oxygen; detecting a first fluorescence intensity of the dye within a selected region of the microfluidic device; flowing a second liquid medium through the channel of the microfluidic device for a second reference period of time, wherein the second liquid medium comprises the dye and a second supplied partial pressure of the oxygen; detecting a second fluorescence intensity of the dye within the selected region of the microfluidic device; and correlating each of the first and the second fluorescence intensities with the first and second supplied partial pressures of the oxygen, respectively. In accordance with various embodiments, the constructing the reference curve further comprises flowing liquid media comprising the dye and successive supplied partial pressures of the oxygen at successive points in time; detecting successive fluorescence intensities of the dye at the successive points in time; and correlating each of the fluorescence intensities with a supplied partial pressure of the oxygen. In accordance with various embodiments, the microfluidic device does not contain any biological micro-objects while constructing the reference curve. In accordance with various embodiments, the selected supplied partial pressure of oxygen is from about 0.02 bar to about 0.21 bar. In accordance with various embodiments, the methods further comprise detecting fluorescence intensities associated with at least three, four, five, or more supplied partial pressures of the oxygen. In accordance with various embodiments, the fluorescence image is taken under a perfusion condition. In accordance with various embodiments, the perfusion condition comprises a constant flow, a steady state flow, a pre-programmed flow with one or more defined flow rates, or zero flow after equilibrium. In accordance with various embodiments, the microfluidic device comprises a plurality of chambers, and the methods further comprise: introducing the population of biological micro-objects into the plurality of chambers. In accordance with various embodiments, the flow region of the microfluidic device comprises a plurality of channels, and the methods further comprise: introducing the population of biological micro-objects into the plurality of channels. In accordance with various embodiments, the flowing the fluidic medium and the taking the fluorescence image are performed at a selected temperature. In accordance with various embodiments, the temperature is from about 20° C. to about 40° C. In accordance with various embodiments, the temperature is from about 28° C. to about 30° C. In accordance with various embodiments, the flowing the fluidic medium and the taking the fluorescence image is performed at a selected pH. In accordance with various embodiments, the pH is from about 3.0 to about 9.0. In accordance with various embodiments, the methods further comprise taking a plurality of fluorescence images at a plurality of timestamps and correlating a respective fluorescence of each fluorescence image to determine a respective observed partial pressure of the oxygen in the AOI at the respective timestamp. In accordance with various embodiments, the methods further comprise taking a plurality of fluorescence images at a plurality of points within the AOL. In accordance with various embodiments, the chamber comprises a sequestration pen, wherein the sequestration comprises an isolation region and a connection region, and wherein the connection region has a proximal opening to the channel and a distal opening to the isolation region. In accordance with various embodiments, the isolation region comprises a single opening to the connection region. In accordance with various embodiments, the population of biological micro-objects is disposed within the isolation region of the sequestration pen. In accordance with various embodiments, the methods further comprise taking a plurality of fluorescence images at a plurality of points extending from the isolation region of the sequestration pen along an axis of diffusion towards the channel. In accordance with various embodiments, the AOI comprises at least part of the connection region. In accordance with various embodiments, the microfluidic device comprises a plurality of exterior surfaces, wherein each of the plurality of exterior surface is oxygen-impermeable. In accordance with various embodiments, the microfluidic device comprises a plurality of exterior surfaces and wherein at least a portion of one or more exterior surfaces of the plurality is coated with an oxygen-impermeable film.

In accordance with various embodiments, the oxygen-impermeable film has an oxygen permeability of at least 1 cm³ mm·m⁻² day⁻¹atm⁻¹. In accordance with various embodiments, the oxygen-impermeable film has an oxygen permeability of at most 20 cm³ mm·m⁻² day⁻¹atm⁻¹. In accordance with various embodiments, the oxygen-impermeable film comprises Parylene N, Parylene C, Parylene D, Parylene HT, epoxy, Torr-Seal epoxy, or any combination thereof. In accordance with various embodiments, the oxygen-impermeable film has a thickness of at least 1 nanometer (nm). In accordance with various embodiments, the oxygen-impermeable film has a thickness of at most 10 micrometers (μm). In accordance with various embodiments, the microfluidic device comprises a plurality of exterior surfaces and the supplied partial pressure of oxygen is delivered to at least a portion of one or more exterior surfaces of the plurality. In accordance with various embodiments, the supplied partial pressure of oxygen is delivered to at least the portion of the one or more exterior surfaces by one or more tubes placed in proximity to the one or more exterior surfaces, the one or more tubes comprising one or more holes (or lumens) configured to allow the supplied partial pressure of oxygen to flow therethrough. In accordance with various embodiments, the supplied partial pressure of oxygen is delivered to the at least the portion of the one or more exterior surfaces by an oxygen bath surrounding the microfluidic device.

In another aspect, a system is provided. In accordance with various embodiments, the system comprises: a microfluidic device comprising: a flow region (e.g., comprising a channel); a chamber configured to receive a population of biological micro-objects therein, wherein the chamber opens to the flow region (or a channel thereof); and a plurality of exterior surfaces, wherein each exterior surface of the plurality is impermeable to oxygen.

In another aspect, a system is provided. In accordance with various embodiments, the system comprises: a microfluidic device comprising: a flow region (e.g., comprising a channel); a chamber configured to receive a population of biological micro-objects therein, wherein the chamber opens to the flow region (or a channel thereof); and a plurality of exterior surfaces, wherein at least a portion of one or more exterior surfaces of the plurality are coated with an oxygen-impermeable film.

In accordance with various embodiments, the oxygen-impermeable film has an oxygen permeability of 20 cm³ mm·m⁻²day⁻¹atm⁻¹ or less (e.g., 15 cm³ mm·m⁻² day⁻¹atm⁻¹ or less, 10 cm³ mm·m⁻² day⁻¹atm⁻¹ or less, or 5 cm³mm·m⁻² day⁻¹atm⁻¹ or less.) In accordance with various embodiments, the oxygen-impermeable film has an oxygen permeability is about 1 cm³ mm·m⁻² day⁻¹atm⁻¹ to about 20 cm³ mm·m⁻² day⁻¹atm⁻¹. In accordance with various embodiments, wherein the oxygen-impermeable film comprises Parylene N, Parylene C, Parylene D, Parylene HT, epoxy, Torr-Seal epoxy, or any combination thereof. In accordance with various embodiments, the oxygen-impermeable film has a thickness of at least 1 nanometer (nm). In accordance with various embodiments, the oxygen-impermeable film has a thickness of at most 10 micrometers (μm). In accordance with various embodiments, the microfluidic device comprises a plurality of chambers, each chamber of the plurality configured to receive one or more (e.g., a population of) biological micro-objects therein. In accordance with various embodiments, the flow region of the microfluidic device comprises a plurality of channels. In accordance with various embodiments, the chamber comprises a sequestration pen, wherein the sequestration pen comprises an isolation region and a connection region, and wherein the connection region has a proximal opening to the flow region (or a channel of the flow region) and a distal opening to the isolation region. In accordance with various embodiments, the isolation region comprises a single opening to the connection region. In accordance with various embodiments, the isolation region of the sequestration pen is configured to receive the population of biological micro-objects therein.

In another aspect, a system is provided. In accordance with various embodiments, the system comprises: an oxygen delivery module; a nest comprising a support structure configured to support a microfluidic device in proximity to the oxygen delivery module; a gas source in fluidic communication with the oxygen delivery module; and a controller configured to control a flow of gas from the gas source to the oxygen delivery module. In accordance with various embodiments, the oxygen delivery module comprises one or more tubes, the one or more tubes comprising one or more holes (or lumens) configured to allow a supplied partial pressure of oxygen to flow therethrough. In accordance with various embodiments, the oxygen delivery module comprises an oxygen bath surrounding the microfluidic device. In accordance with various embodiments, the nest is configured to provide a fluidic connection between the system and said microfluidic device. In accordance with various embodiments, the nest further comprises a socket configured to provide an electrical interface between the system and said microfluidic device. In accordance with various embodiments, the system further comprises a fluidic medium source comprising a sparging component in fluidic communication with the gas source. In accordance with various embodiments, the system further comprises a structured light source positioned to direct structured light to said microfluidic device and configured to thereby activate phototransistors within said microfluidic device. In accordance with various embodiments, the system further comprises a microfluidic device disposed on the support structure, the microfluidic device comprising: a flow region (e.g., a channel); and a chamber configured to receive a population of biological micro-objects therein, wherein the chamber opens to the flow region. In accordance with various embodiments, the microfluidic device comprises a plurality of chambers, each chamber of the plurality configured to receive a population of biological micro-objects therein. In accordance with various embodiments, the flow region of the microfluidic device comprises a plurality of channels. In accordance with various embodiments, the chamber comprises a sequestration pen, wherein the sequestration comprises an isolation region and a connection region, and wherein the connection region has a proximal opening to the flow region (or a channel of the flow region) and a distal opening to the isolation region. In accordance with various embodiments, the isolation region comprises a single opening to the connection region.

In another aspect, a kit is provided. In accordance with various embodiments, the kit comprises: a microfluidic device comprising: a flow region (e.g., a channel); a chamber configured to receive a population of biological micro-objects therein, wherein the chamber opens to the flow region (or a channel of the flow region); and a plurality of exterior surfaces, wherein each exterior surface of the plurality is impermeable to oxygen or wherein at least a portion of one or more exterior surfaces of the plurality are coated with an oxygen-impermeable film; and a buffer. In accordance with various embodiments, the kit further comprises a fluidic medium containing a dye configured to emit a fluorescence signal that changes based on availability of oxygen proximate to the dye. In accordance with various embodiments, the dye comprises a soluble and diffusible dye. In accordance with various embodiments, the dye comprises a ruthenium complex.

In another aspect, a kit is provided. In accordance with various embodiments, the kit comprises: a microfluidic device comprising: a flow region (e.g., a channel); a chamber configured to receive a population of biological micro-objects therein, wherein the chamber opens to the flow region (or a channel of the flow region); and a plurality of exterior surfaces, wherein each exterior surface of the plurality is impermeable to oxygen or wherein at least a portion of one or more exterior surfaces of the plurality are coated with an oxygen-impermeable film; and a fluidic medium containing a dye configured to emit a fluorescence signal that changes based on availability of oxygen proximate to the dye. In accordance with various embodiments, the dye comprises a soluble and diffusible dye. In accordance with various embodiments, the dye comprises a ruthenium complex. In accordance with various embodiments, the kit further comprises a buffer.

Additional methods and systems are provided in the claims attached hereto.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a more complete understanding of the principles disclosed herein, and the advantages thereof, reference is now made to the following descriptions taken in conjunction with the accompanying drawings, in which:

FIG. 1A illustrates a microfluidic device and a system with associated control equipment according to some embodiments of the disclosure.

FIG. 1B illustrates a microfluidic device with sequestration pens according to an embodiment of the disclosure.

FIGS. 2A-2B illustrate a microfluidic device having sequestration pens according to some embodiments of the disclosure.

FIG. 2A depicts a vertical cross-section of microfluidic device according to some embodiments of the disclosure.

FIG. 2B shows a horizontal cross-section of microfluidic device according to some embodiments of the disclosure.

FIG. 2C illustrates a sequestration pen of a microfluidic device according to some embodiments of the disclosure.

FIG. 3 illustrates a sequestration pen of a microfluidic device according to some embodiments of the disclosure.

FIGS. 4A-4B illustrate electrokinetic features of a microfluidic device according to some embodiments of the disclosure.

FIG. 4A shows a side cross-sectional view of a portion of an enclosure of the microfluidic device according to some embodiments of the disclosure.

FIG. 4B shows a top cross-sectional view of a portion of an enclosure of the microfluidic device according to some embodiments of the disclosure.

FIG. 5A illustrates a system for use with a microfluidic device and associated control equipment according to some embodiments of the disclosure.

FIG. 5B illustrates an imaging device according to some embodiments of the disclosure.

FIG. 6 illustrates heatmaps of normalized fluorescence intensity categorized by perfusion conditions at various timestamps of a microfluidic device according to some embodiments of the disclosure.

FIG. 7A illustrates heatmaps of normalized oxygen level/consumption at a specified perfusion condition at various timestamps of a microfluidic device according to some embodiments of the disclosure.

FIG. 7B illustrates heatmaps of normalized oxygen level/consumption at various perfusion conditions at a fixed perfusion period of time of a microfluidic device according to some embodiments of the disclosure.

FIG. 8 is a graphical representation showing normalized oxygen level/consumption as a function of biomass (e.g., a population of biological micro-objects) for sequestration pens of a microfluidic device according to some embodiments of the disclosure.

FIGS. 9A-9B illustrate normalized fluorescence intensity as a function of oxygen level in channels of a microfluidic device according to some embodiments of the disclosure.

FIG. 9A shows a plot of normalized fluorescence intensity taken during standard perfusion in accordance with some embodiments of the present disclosure.

FIG. 9B shows a plot of normalized fluorescence intensity taken without perfusion in accordance with some embodiments of the present disclosure.

FIGS. 9C-9D illustrate normalized fluorescence intensity as a function of oxygen level in sequestration pens of a microfluidic device according to some embodiments of the disclosure.

FIG. 9C shows a plot of normalized fluorescence intensity taken during standard perfusion in accordance with some embodiments of the present disclosure.

FIG. 9D shows a plot of normalized fluorescence intensity taken without perfusion in accordance with some embodiments of the present disclosure.

FIG. 10 illustrates a flow chart for an example method of determining oxygen consumption level in a population of biological micro-objects in sequestrations pens according to various embodiments of the present disclosure.

FIG. 11 illustrates an example approach of converting acquired fluorescence images into data for correlating fluorescence of an AOI to a reference to determine the dissolved oxygen level in accordance with some embodiments of the present disclosure.

FIG. 12A illustrates a first example approach of generating a dissolved oxygen (DO) standard curve in accordance with some embodiments of the present disclosure.

FIG. 12B illustrates a second example approach of generating a DO standard curve in accordance with some embodiments of the present disclosure.

FIG. 13 illustrates exemplary DO standard curves generated by the process of FIG. 12A in accordance with some embodiments of the present disclosure.

FIG. 14 illustrates a first example approach of performing a DO perfusion assay in accordance with some embodiments of the present disclosure.

FIG. 15 illustrates a second example approach of performing a DO perfusion assay in accordance with some embodiments of the present disclosure.

FIG. 16 shows an oxygen delivery system comprising one or more tubes with one or more holes (or lumens) in accordance with some embodiments of the present disclosure.

FIG. 17 shows the variability of the normalized fluorescence intensity at a 0.4 mg/mL dye concentration in accordance with some embodiments of the present disclosure.

FIGS. 18A-18B show the improvement in dissolved oxygen uniformity achieved by sealing the microfluidic chip from external gas exchange using Parylene in accordance with some embodiments of the present disclosure.

FIG. 18A shows an exemplary average normalized intensity of dissolved oxygen fluorescence signal in a sequestration pen of a Parylene-sealed microfluidic device in accordance with some embodiments of the present disclosure.

FIG. 18B shows an exemplary average normalized intensity of dissolved oxygen fluorescence signal in a channel of a Parylene-sealed microfluidic device in accordance with some embodiments of the present disclosure.

FIGS. 19A-19B show the different performance levels of various sealing techniques in limiting external gas exchange to improve dissolved oxygen uniformity in accordance with some embodiments of the present disclosure.

FIG. 19A shows an exemplary average normalized intensity of dissolved oxygen fluorescence signal in sequestration pens of microfluidic chips sealed using Torr-Seal, placed in a gas bath, and sealed using Parylene in accordance with some embodiments of the present disclosure.

FIG. 19B shows an exemplary average normalized intensity of dissolved oxygen fluorescence signal in channels of microfluidic chips sealed using Torr-Seal, placed in a gas bath, and sealed using Parylene in accordance with some embodiments of the present disclosure.

FIGS. 20A-20B show an example of how the above-described non-uniformities in external gas exchange impact the dissolved oxygen signal as observed over the whole chip in accordance with some embodiments of the present disclosure.

FIG. 20A shows an exemplary uniformity of dissolved oxygen fluorescence signal across sequestration pens of microfluidic chips sealed using Torr-Seal, place in a gas bath, and sealed using Parylene in accordance with some embodiments of the present disclosure.

FIG. 20B shows an exemplary uniformity of dissolved oxygen fluorescence signal across channels of microfluidic chips sealed using Torr-Seal, placed in a gas bath, and sealed using Parylene in accordance with some embodiments of the present disclosure.

FIG. 21A shows an exemplary brightfield image of cells in sequestration pens in accordance with some embodiments of the present disclosure.

FIG. 21B shows an exemplary fluorescence image of dissolved oxygen in the sequestration pens in accordance with some embodiments of the present disclosure.

FIG. 22 shows a system configured to implement the methods described herein in accordance with some embodiments of the present disclosure.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Where dimensions of microfluidic features are described as having a width or an area, the dimension typically is described relative to an x-axial and/or y-axial dimension, both of which lie within a plane that is parallel to the substrate and/or cover of the microfluidic device. The height of a microfluidic feature may be described relative to a z-axial direction, which is perpendicular to a plane that is parallel to the substrate and/or cover of the microfluidic device. In some instances, a cross sectional area of a microfluidic feature, such as a channel or a passageway, may be in reference to a x-axial/z-axial, a y-axial/z-axial, or an x-axial/y-axial area.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one. As used herein, the term “plurality” can be 2, 3, 4, 5, 6, 7, 8, 9, 10, or more.

As used herein: μm means micrometer, μm³ means cubic micrometer, pL means picoliter, nL means nanoliter, and μL (or uL) means microliter.

As used herein, “air” refers to the composition of gases predominating in the atmosphere of the earth. The four most plentiful gases are nitrogen (typically present at a concentration of about 78% by volume, e.g., in a range from about 70-80%), oxygen (typically present at about 20.95% by volume at sea level, e.g. in a range from about 10% to about 25%), argon (typically present at about 1.0% by volume, e.g. in a range from about 0.1% to about 3%), and carbon dioxide (typically present at about 0.04%, e.g., in a range from about 0.01% to about 0.07%). Air may have other trace gases such as methane, nitrous oxide or ozone, trace pollutants and organic materials such as pollen, diesel particulates and the like. Air may include water vapor (typically present at about 0.25% or may be present in a range from about 10 ppm to about 5% by volume). Air may be provided for use in culturing experiments as a filtered, controlled composition and may be conditioned as described herein.

As used herein, the term “disposed” encompasses within its meaning “located.”

As used herein, a “microfluidic device” or “microfluidic apparatus” is a device that includes one or more discrete microfluidic circuits configured to hold a fluid, each microfluidic circuit comprised of fluidically interconnected circuit elements, including but not limited to region(s), flow path(s), channel(s), chamber(s), and/or pen(s), and at least one port configured to allow the fluid (and, optionally, micro-objects suspended in the fluid) to flow into and/or out of the microfluidic device. Typically, a microfluidic circuit of a microfluidic device will include a flow region, which may include a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 μL. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 μL. The microfluidic circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of microfluidic device having a microfluidic circuit that contains at least one circuit element configured to hold a volume of fluid of less than about 1 μL, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 pL to 1 nL, 100 pL to 2 nL, 100 pL to 5 nL, 250 pL to 2 nL, 250 pL to 5 nL, 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, one or more (e.g., all) of the at least one circuit elements are configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A microfluidic device or a nanofluidic device may be referred to herein as a “microfluidic chip” or a “chip”; or “nanofluidic chip” or “chip”.

A “microfluidic channel” or “flow channel” as used herein refers to flow region of a microfluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is about 100,000 microns to about 500,000 microns, including any value therebetween. In some embodiments, the horizontal dimension is about 100 microns to about 1000 microns (e.g., about 150 to about 500 microns) and the vertical dimension is about 25 microns to about 200 microns, (e.g., from about 40 to about 150 microns). It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein. The flow channel may include valves, and the valves may be of any type known in the art of microfluidics. Examples of microfluidic channels that include valves are disclosed in U.S. Pat. Nos. 6,408,878 and 9,227,200, each of which is herein incorporated by reference in its entirety.

As used herein, the term “obstruction” refers generally to a bump or similar type of structure that is sufficiently large so as to partially (but not completely) impede movement of target micro-objects between two different regions or circuit elements in a microfluidic device. The two different regions/circuit elements can be, for example, a microfluidic sequestration pen and a microfluidic channel, or a connection region and an isolation region of a microfluidic sequestration pen.

As used herein, the term “constriction” refers generally to a narrowing of a width of a circuit element (or an interface between two circuit elements) in a microfluidic device. The constriction can be located, for example, at the interface between a microfluidic sequestration pen and a microfluidic channel, or at the interface between an isolation region and a connection region of a microfluidic sequestration pen.

As used herein, the term “transparent” refers to a material which allows visible light to pass through without substantially altering the light as is passes through.

As used herein, “brightfield” illumination and/or image refers to white light illumination of the microfluidic field of view from a broad-spectrum light source, where contrast is formed by absorbance of light by objects in the field of view.

As used herein, “structured light” is projected light that is modulated to provide one or more illumination effects. A first illumination effect may be projected light illuminating a portion of a surface of a device without illuminating (or at least minimizing illumination of) an adjacent portion of the surface, e.g., a projected light pattern, as described more fully below, used to activate DEP forces within a DEP substrate. When using structured light patterns to activate DEP forces, the intensity, e.g., variation in duty cycle of a structured light modulator such as a DMD, may be used to change the optical power applied to the light activated DEP actuators, and thus change DEP force without changing the nominal voltage or frequency. Another illumination effect that may be produced by structured light includes projected light that may be corrected for surface irregularities and for irregularities associated with the light projection itself, e.g., fall-off at the edge of an illuminated field. Structured light is typically generated by a structured light modulator, such as a digital mirror device (DMD), a microshutter array system (MSA), a liquid crystal display (LCD), or the like. Illumination of a small area of the surface, e.g., a selected area of interest, with structured light improves the signal-to-noise-ratio (SNR), as illumination of only the selected area of interest reduces stray/scattered light, thereby lowering the dark level of the image. An important aspect of structured light is that it may be changed quickly over time. A light pattern from the structured light modulator, e.g., DMD, may be used to autofocus on difficult targets such as clean mirrors or surfaces that are far out of focus. Using a clean mirror, a number of self-test features may be replicated such as measurement of modulation transfer function and field curvature/tilt, without requiring a more expensive Shack-Hartmann sensor. In another use of structured light patterns, spatial power distribution may be measured at the sample surface with a simple power meter, in place of a camera. Structured light patterns may also be used as a reference feature for optical module/system component alignment as well used as a manual readout for manual focus. Another illumination effect made possible by use of structured light patterns is selective curing, e.g., solidification of hydrogels within the microfluidic device.

As used herein, the term “micro-object” refers generally to any microscopic object that may be isolated and/or manipulated in accordance with the present disclosure. Non-limiting examples of micro-objects include: inanimate micro-objects such as microparticles; microbeads (e.g., polystyrene beads, glass beads, amorphous solid substrates, Luminex™ beads, or the like); magnetic beads; microrods; microwires; quantum dots, and the like; biological micro-objects such as cells; biological organelles; vesicles, or complexes; synthetic vesicles; liposomes (e.g., synthetic or derived from membrane preparations); lipid nanorafts, and the like; or a combination of inanimate micro-objects and biological micro-objects (e.g., microbeads attached to cells, liposome-coated micro-beads, liposome-coated magnetic beads, or the like). Beads may include moieties/molecules covalently or non-covalently attached, such as fluorescence labels, proteins (including receptor molecules), carbohydrates, antigens, small molecule signaling moieties, or other chemical/biological species capable of use in an assay. In some variations, beads/solid substrates including moieties/molecules may be capture beads, e.g., configured to bind molecules including small molecules, peptides, proteins or nucleic acids present in proximity either selectively or nonselectively. In one nonlimiting example, a capture bead may include a nucleic acid sequence configured to bind nucleic acids having a specific nucleic acid sequence or the nucleic acid sequence of the capture bead may be configured to bind a set of nucleic acids having related nucleic acid sequences. Either type of binding may be understood to be selective. Capture beads containing moieties/molecules may bind nonselectively when binding of structurally different but physico-chemically similar molecules is performed, for example, size exclusion beads or zeolites configured to capture molecules of selected size or charge. Lipid nanorafts have been described, for example, in Ritchie et al. (2009) “Reconstitution of Membrane Proteins in Phospholipid Bilayer Nanodiscs,” Methods Enzymol., 464:211-231.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g., about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the term “maintaining (a) cell(s)” refers to providing an environment comprising both fluidic and gaseous components and, optionally a surface, that provides the conditions necessary to keep the cells viable and/or expanding.

As used herein, the term “expanding” when referring to cells, refers to increasing in cell number.

As referred to herein, “gas permeable” means that the material or structure is permeable to at least one of oxygen, carbon dioxide, or nitrogen. In some embodiments, the gas permeable material or structure is permeable to more than one of oxygen, carbon dioxide and nitrogen and may further be permeable to all three of these gases.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion, and may encompass perfusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result. Flowing can comprise pulling solution through and out of the microfluidic channel (e.g., aspirating) or pushing fluid into and through a microfluidic channel (e.g., perfusing).

The phrase “substantially no flow” refers to a rate of flow of a fluidic medium that, when averaged over time, is less than the rate of diffusion of components of a material (e.g., an analyte of interest) into or within the fluidic medium. The ratio of a rate of flow of a component in a fluidic medium (i.e., advection) divided by the rate of diffusion of such component can be expressed by a dimensionless Peclet number. Thus, a region within a microfluidic device that experiences substantially no flow in one in which the Peclet number is less than 1. The Peclet number associated with a particular region within the microfluidic device can vary with the component or components of the fluidic medium being considered (e.g., the analyte of interest), as the rate of diffusion of a component or components in a fluidic medium can depend on, for example, temperature, the size, mass, and/or shape of the component(s), and the strength of interactions between the component(s) and the fluidic medium. In certain embodiments, the Peclet number associated with a particular region of the microfluidic device and a component located therein can be 0.95 or less, 0.9 or less, 0.85 or less, 0.8 or less, 0.75 or less, 0.7 or less, 0.65 or less, 0.6 or less, 0.55 or less, 0.5 or less, 0.4 or less, 0.3 or less, 0.2 or less, 0.1 or less, 0.05 or less, 0.01 or less, 0.005 or less, or 0.001 or less.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the device.

As used herein, a “flow path” refers to one or more fluidically connected circuit elements (e.g., channel(s), region(s), chamber(s) and the like) that define, and are subject to, the trajectory of a flow of medium. A flow path is thus an example of a swept region of a microfluidic device. Other circuit elements (e.g., unswept regions) may be fluidically connected with the circuit elements that comprise the flow path without being subject to the flow of medium in the flow path.

As used herein, “isolating a micro-object” confines a micro-object to a defined area within the microfluidic device.

As used herein, “pen” or “penning” refers to disposing micro-objects within a chamber (e.g., a sequestration pen) within the microfluidic device. Forces used to pen a micro-object may be any suitable force as described herein such as dielectrophoresis (DEP), e.g., an optically actuated dielectrophoretic force (OEP); gravity; magnetic forces; or tilting. In some embodiments, penning a plurality of micro-objects may reposition substantially all the micro-objects. In some other embodiments, a selected number of the plurality of micro-objects may be penned, and the remainder of the plurality may not be penned. In some embodiments, when selected micro-objects are penned, a DEP force, e.g., an optically actuated DEP force or a magnetic force may be used to reposition the selected micro-objects. Typically, micro-objects may be introduced to a flow region, e.g., a microfluidic channel, of the microfluidic device and introduced into a chamber by penning.

As used herein, “unpen” or “unpenning” refers to repositioning micro-objects from within a chamber, e.g., a sequestration pen, to a new location within a flow region, e.g., a microfluidic channel, of the microfluidic device. Forces used to unpen a micro-object may be any suitable force as described herein such as dielectrophoresis, e.g., an optically actuated dielectrophoretic force; gravity; magnetic forces; or tilting. In some embodiments, unpenning a plurality of micro-objects may reposition substantially all the micro-objects. In some other embodiments, a selected number of the plurality of micro-objects may be unpenned, and the remainder of the plurality may not be unpenned. In some embodiments, when selected micro-objects are unpenned, a DEP force, e.g., an optically actuated DEP force or a magnetic force may be used to reposition the selected micro-objects.

As used herein, “export” or “exporting” refers to repositioning micro-objects from a location within a flow region, e.g., a microfluidic channel, of a microfluidic device to a location outside of the microfluidic device, such as a 96 well plate or other receiving vessel. The orientation of the chamber(s) having an opening to the microfluidic channel permits easy export of micro-objects that have been positioned or repositioned (e.g., unpenned from a chamber) to be disposed within the microfluidic channel. Micro-objects within the microfluidic channel may be exported without requiring disassembly (e.g., removal of the cover of the device) or insertion of a tool into the chamber(s) or microfluidic channel to remove micro-objects for further processing.

A microfluidic (or nanofluidic) device can comprise “swept” regions and “unswept” regions. As used herein, a “swept” region is comprised of one or more fluidically interconnected circuit elements of a microfluidic circuit, each of which experiences a flow of medium when fluid is flowing through the microfluidic circuit. The circuit elements of a swept region can include, for example, regions, channels, and all or parts of chambers. As used herein, an “unswept” region is comprised of one or more fluidically interconnected circuit element of a microfluidic circuit, each of which experiences substantially no flux of fluid when fluid is flowing through the microfluidic circuit. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region. For example, a flow channel of a micro-fluidic device is an example of a swept region while an isolation region (described in further detail below) of a microfluidic device is an example of an unswept region.

As used herein, a “non-sweeping” rate of fluidic medium flow means a rate of flow sufficient to permit components of a second fluidic medium in an isolation region of the sequestration pen to diffuse into the first fluidic medium in the flow region and/or components of the first fluidic medium to diffuse into the second fluidic medium in the isolation region; and further wherein the first medium does not substantially flow into the isolation region.

As used herein, “capture moiety” is a chemical or biological species, functionality, or motif that provides a recognition site for a micro-object. A selected class of micro-objects may recognize the in situ-generated capture moiety and may bind or have an affinity for the in situ-generated capture moiety. Non-limiting examples include antigens, antibodies, and cell surface binding motifs.

As used herein, “antibody” refers to an immunoglobulin (Ig) and includes both polyclonal and monoclonal antibodies; primatized (e.g., humanized); murine; mouse-human; mouse-primate; and chimeric; and may be an intact molecule, a fragment thereof (such as scFv, Fv, Fd, Fab, Fab′ and F(ab)′2 fragments), or multimers or aggregates of intact molecules and/or fragments; and may occur in nature or be produced, e.g., by immunization, synthesis or genetic engineering. An “antibody fragment,” as used herein, refers to fragments, derived from or related to an antibody, which bind antigen and which in some embodiments may be derivatized to exhibit structural features that facilitate clearance and uptake, e.g., by the incorporation of galactose residues. This includes, e.g., F(ab), F(ab)′2, scFv, light chain variable region (VL), heavy chain variable region (VH), and combinations thereof.

The capability of biological micro-objects (e.g., biological cells) to produce specific biological materials (e.g., proteins, such as antibodies) can be assayed in such a microfluidic device. In a specific embodiment of an assay, sample material comprising biological micro-objects (e.g., cells) to be assayed for production of an analyte of interest (e.g., a biomolecule of interest) can be loaded into a swept region of the microfluidic device. Ones of the biological micro-objects (e.g., mammalian cells, such as human cells) can be selected for particular characteristics and disposed in unswept regions. The remaining sample material can then be flowed out of the swept region and an assay material flowed into the swept region. Because the selected biological micro-objects are in unswept regions, the selected biological micro-objects are not substantially affected by the flowing out of the remaining sample material or the flowing in of the assay material. The selected biological micro-objects can be allowed to produce the analyte of interest, which can diffuse from the unswept regions into the swept region, where the analyte of interest can react with the assay material to produce localized detectable reactions, each of which can be correlated to a particular unswept region. Any unswept region associated with a detected reaction can be analyzed to assess which, if any, of the biological micro-objects in the unswept region are sufficient producers of the analyte of interest.

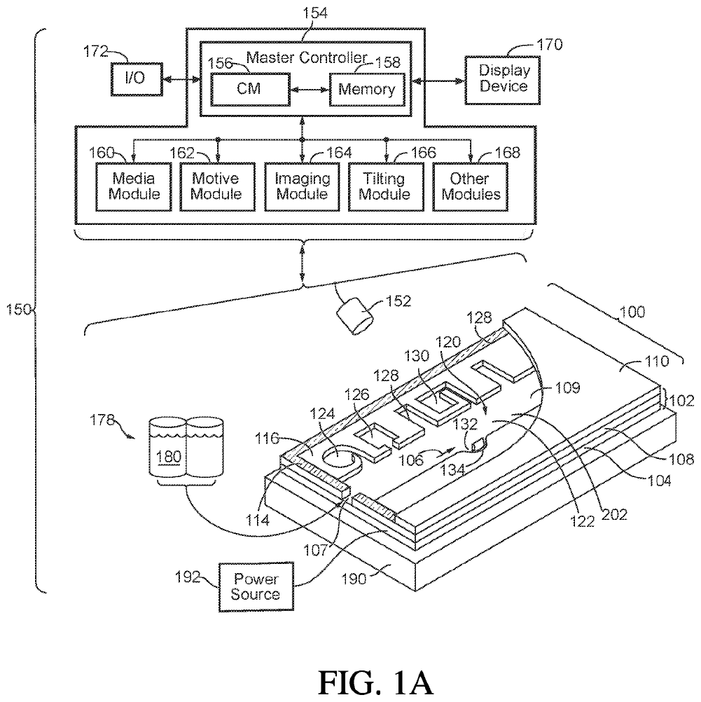

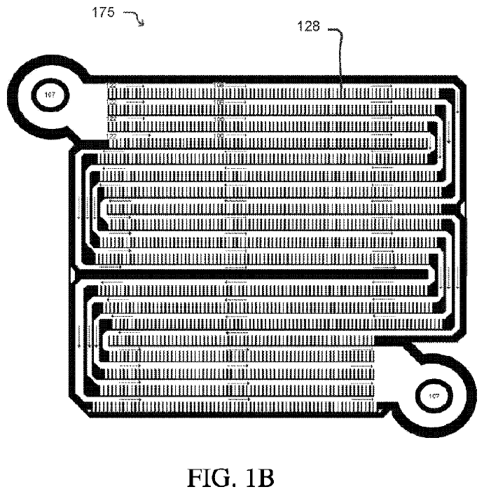

Microfluidic devices/systems featuring cross-applicability. It should be appreciated that various features of microfluidic devices, systems, and motive technologies described herein may be combinable or interchangeable. For example, features described herein with reference to the microfluidic device 100, 175, 200, 300, 320, 400, 450, 520 and system attributes as described in FIGS. 1A-5B may be combinable or interchangeable.

Microfluidic devices. FIG. 1A illustrates an example of a microfluidic device 100. A perspective view of the microfluidic device 100 is shown having a partial cut-away of its cover 110 to provide a partial view into the microfluidic device 100. The microfluidic device 100 generally comprises a microfluidic circuit 120 comprising a flow path 106 through which a fluidic medium 180 can flow, optionally carrying one or more micro-objects (not shown) into and/or through the microfluidic circuit 120.

As generally illustrated in FIG. 1A, the microfluidic circuit 120 is defined by an enclosure 102. Although the enclosure 102 can be physically structured in different configurations, in the example shown in FIG. 1A the enclosure 102 is depicted as comprising a support structure 104 (e.g., a base), a microfluidic circuit structure 108, and a cover 110. The support structure 104, microfluidic circuit structure 108, and cover 110 can be attached to each other. For example, the microfluidic circuit structure 108 can be disposed on an inner surface 109 of the support structure 104, and the cover 110 can be disposed over the microfluidic circuit structure 108. Together with the support structure 104 and cover 110, the microfluidic circuit structure 108 can define the elements of the microfluidic circuit 120, forming a three-layer structure.

The support structure 104 can be at the bottom and the cover 110 at the top of the microfluidic circuit 120 as illustrated in FIG. 1A. Alternatively, the support structure 104 and the cover 110 can be configured in other orientations. For example, the support structure 104 can be at the top and the cover 110 at the bottom of the microfluidic circuit 120. Regardless, there can be one or more ports 107 each comprising a passage into or out of the enclosure 102. Examples of a passage include a valve, a gate, a pass-through hole, or the like. As illustrated, port 107 is a pass-through hole created by a gap in the microfluidic circuit structure 108. However, the port 107 can be situated in other components of the enclosure 102, such as the cover 110. Only one port 107 is illustrated in FIG. 1A but the microfluidic circuit 120 can have two or more ports 107. For example, there can be a first port 107 that functions as an inlet for fluid entering the microfluidic circuit 120, and there can be a second port 107 that functions as an outlet for fluid exiting the microfluidic circuit 120. Whether a port 107 function as an inlet or an outlet can depend upon the direction that fluid flows through flow path 106.

The support structure 104 can comprise one or more electrodes (not shown) and a substrate or a plurality of interconnected substrates. For example, the support structure 104 can comprise one or more semiconductor substrates, each of which is electrically connected to an electrode (e.g., all or a subset of the semiconductor substrates can be electrically connected to a single electrode). The support structure 104 can further comprise a printed circuit board assembly (“PCBA”). For example, the semiconductor substrate(s) can be mounted on a PCBA.

The microfluidic circuit structure 108 can define circuit elements of the microfluidic circuit 120. Such circuit elements can comprise spaces or regions that can be fluidly interconnected when microfluidic circuit 120 is filled with fluid, such as flow regions (which may include or be one or more flow channels), chambers (which class of circuit elements may also include sub-classes including sequestration pens), traps, and the like. Circuit elements can also include barriers, and the like. In the microfluidic circuit 120 illustrated in FIG. 1A, the microfluidic circuit structure 108 comprises a frame 114 and a microfluidic circuit material 116. The frame 114 can partially or completely enclose the microfluidic circuit material 116. The frame 114 can be, for example, a relatively rigid structure substantially surrounding the microfluidic circuit material 116. For example, the frame 114 can comprise a metal material. However, the microfluidic circuit structure need not include a frame 114. For example, the microfluidic circuit structure can consist of (or consist essentially of) the microfluidic circuit material 116.

The microfluidic circuit material 116 can be patterned with cavities or the like to define the circuit elements and interconnections of the microfluidic circuit 120, such as chambers, pens and microfluidic channels. The microfluidic circuit material 116 can comprise a flexible material, such as a flexible polymer (e.g., rubber, plastic, elastomer, silicone, polydimethylsiloxane (“PDMS”), or the like), which can be gas permeable. Other examples of materials that can form the microfluidic circuit material 116 include molded glass, an etchable material such as silicone (e.g., photo-patternable silicone or “PPS”), photo-resist (e.g., SU8), or the like. In some embodiments, such materials—and thus the microfluidic circuit material 116—can be rigid and/or substantially impermeable to gas. Regardless, microfluidic circuit material 116 can be disposed on the support structure 104 and inside the frame 114.

The microfluidic circuit 120 can include a flow region in which one or more chambers can be disposed and/or fluidically connected thereto. A chamber can have one or more openings fluidically connecting the chamber with one or more flow regions. In some embodiments, a flow region comprises or corresponds to a microfluidic channel 122. Although a single microfluidic circuit 120 is illustrated in FIG. 1A, suitable microfluidic devices can include a plurality (e.g., 2 or 3) of such microfluidic circuits. In some embodiments, the microfluidic device 100 can be configured to be a nanofluidic device. As illustrated in FIG. 1A, the microfluidic circuit 120 may include a plurality of microfluidic sequestration pens 124, 126, 128, and 130, where each sequestration pens may have one or more openings. In some embodiments of sequestration pens, a sequestration pen may have only a single opening in fluidic communication with the flow path 106. In some other embodiments, a sequestration pen may have more than one opening in fluidic communication with the flow path 106, e.g., n number of openings, but with n−1 openings that are valved, such that all but one opening is closable. When all the valved openings are closed, the sequestration pen limits exchange of materials from the flow region into the sequestration pen to occur only by diffusion. In some embodiments, the sequestration pens comprise various features and structures (e.g., isolation regions) that have been optimized for retaining micro-objects within the sequestration pen (and therefore within a microfluidic device such as microfluidic device 100) even when a medium 180 is flowing through the flow path 106.

The cover 110 can be an integral part of the frame 114 and/or the microfluidic circuit material 116. Alternatively, the cover 110 can be a structurally distinct element, as illustrated in FIG. 1A. The cover 110 can comprise the same or different materials than the frame 114 and/or the microfluidic circuit material 116. In some embodiments, the cover 110 can be an integral part of the microfluidic circuit material 116. Similarly, the support structure 104 can be a separate structure from the frame 114 or microfluidic circuit material 116 as illustrated, or an integral part of the frame 114 or microfluidic circuit material 116. Likewise, the frame 114 and microfluidic circuit material 116 can be separate structures as shown in FIG. 1A or integral portions of the same structure. Regardless of the various possible integrations, the microfluidic device can retain a three-layer structure that includes a base layer and a cover layer that sandwich a middle layer in which the microfluidic circuit 120 is located.

In some embodiments, the cover 110 can comprise a rigid material. The rigid material may be glass or a material with similar properties. In some embodiments, the cover 110 can comprise a deformable material. The deformable material can be a polymer, such as PDMS. In some embodiments, the cover 110 can comprise both rigid and deformable materials. For example, one or more portions of cover 110 (e.g., one or more portions positioned over sequestration pens 124, 126, 128, 130) can comprise a deformable material that interfaces with rigid materials of the cover 110. Microfluidic devices having covers that include both rigid and deformable materials have been described, for example, in U.S. Pat. No. 10,058,865 (Breinlinger et al.), the contents of which are incorporated herein by reference. In some embodiments, the cover 110 can further include one or more electrodes. The one or more electrodes can comprise a conductive oxide, such as indium-tin-oxide (ITO), which may be coated on glass or a similarly insulating material. Alternatively, the one or more electrodes can be flexible electrodes, such as single-walled nanotubes, multi-walled nanotubes, nanowires, clusters of electrically conductive nanoparticles, or combinations thereof, embedded in a deformable material, such as a polymer (e.g., PDMS). Flexible electrodes that can be used in microfluidic devices have been described, for example, in U.S. Pat. No. 9,227,200 (Chiou et al.), the contents of which are incorporated herein by reference. In some embodiments, the cover 110 and/or the support structure 104 can be transparent to light. The cover 110 may also include at least one material that is gas permeable (e.g., PDMS or PPS).

In the example shown in FIG. 1A, the microfluidic circuit 120 is illustrated as comprising a microfluidic channel 122 and sequestration pens 124, 126, 128, 130. Each pen comprises an opening to channel 122, but otherwise is enclosed such that the pens can substantially isolate micro-objects inside the pen from fluidic medium 180 and/or micro-objects in the flow path 106 of channel 122 or in other pens. The walls of the sequestration pen extend from the inner surface 109 of the base to the inside surface of the cover 110 to provide enclosure. The opening of the sequestration pen to the microfluidic channel 122 is oriented at an angle to the flow 106 of fluidic medium 180 such that flow 106 is not directed into the pens. The vector of bulk fluid flow in channel 122 may be tangential or parallel to the plane of the opening of the sequestration pen, and is not directed into the opening of the pen. In some instances, pens 124, 126, 128, 130 are configured to physically isolate one or more micro-objects within the microfluidic circuit 120. Sequestration pens in accordance with the present disclosure can comprise various shapes, surfaces and features that are optimized for use with DEP, OET, OEW, fluid flow, magnetic forces, centripetal, and/or gravitational forces, as will be discussed and shown in detail below.

The microfluidic circuit 120 may comprise any number of microfluidic sequestration pens. Although five sequestration pens are shown, microfluidic circuit 120 may have fewer or more sequestration pens. As shown, microfluidic sequestration pens 124, 126, 128, and 130 of microfluidic circuit 120 each comprise differing features and shapes which may provide one or more benefits useful for maintaining, isolating, assaying or culturing biological micro-objects. In some embodiments, the microfluidic circuit 120 comprises a plurality of identical microfluidic sequestration pens.

In the embodiment illustrated in FIG. 1A, a single flow path 106 containing a single channel 122 is shown. However, other embodiments may contain multiple channels 122 within a single flow path 106, as shown in FIG. 1B. The microfluidic circuit 120 further comprises an inlet valve or port 107 in fluid communication with the flow path 106, whereby fluidic medium 180 can access the flow path 106 (and channel 122). In some instances, the flow path 106 comprises a substantially straight path. In other instances, the flow path 106 is arranged in a non-linear or winding manner, such as a zigzag pattern, whereby the flow path 106 travels across the microfluidic device 100 two or more times, e.g., in alternating directions. The flow in the flow path 106 may proceed from inlet to outlet or may be reversed and proceed from outlet to inlet.

One example of a multi-channel device, microfluidic device 175, is shown in FIG. 1B, which may be like microfluidic device 100 in other respects. Microfluidic device 175 and its constituent circuit elements (e.g., channels 122 and sequestration pens 128) may have any of the dimensions discussed herein. The microfluidic circuit illustrated in FIG. 1B has two inlet/outlet ports 107 and a flow path 106 containing four distinct channels 122. The number of channels into which the microfluidic circuit is sub-divided may be chosen to reduce fluidic resistance. For example, the microfluidic circuit may include 1, 2, 3, 4, 5, 6, 7, 8, 9, 10, or more channels to provide a selected range of fluidic resistance. Microfluidic device 175 further comprises a plurality of sequestration pens opening off of each channel 122, where each of the sequestration pens is similar to sequestration pen 128 of FIG. 1A, and may have any of the dimensions or functions of any sequestration pen as described herein. However, the sequestration pens of microfluidic device 175 can have different shapes, such as any of the shapes of sequestration pens 124, 126, or 130 of FIG. 1A or as described anywhere else herein. Moreover, microfluidic device 175 can include sequestration pens having a mixture of different shapes. In some instances, a plurality of sequestration pens is configured (e.g., relative to a channel 122) such that the sequestration pens can be loaded with target micro-objects in parallel.

Returning to FIG. 1A, microfluidic circuit 120 further may include one or more optional micro-object traps 132. The optional traps 132 may be formed in a wall forming the boundary of a channel 122, and may be positioned opposite an opening of one or more of the microfluidic sequestration pens 124, 126, 128, 130. The optional traps 132 may be configured to receive or capture a single micro-object from the flow path 106, or may be configured to receive or capture a plurality of micro-objects from the flow path 106. In some instances, the optional traps 132 comprise a volume approximately equal to the volume of a single target micro-object. In some instances, the trap 132 comprises a side passage 134 that is smaller than the target micro-object in order to facilitate flow through the trap 132.

Sequestration pens. The microfluidic devices described herein may include one or more sequestration pens, where each sequestration pen is suitable for holding one or more micro-objects (e.g., biological cells, or groups of cells that are associated together). The sequestration pens may be disposed within and open to a flow region, which in some embodiments is a microfluidic channel. Each of the sequestration pens can have one or more openings for fluidic communication to one or more microfluidic channels. In some embodiments, a sequestration pen may have only one opening to a microfluidic channel.

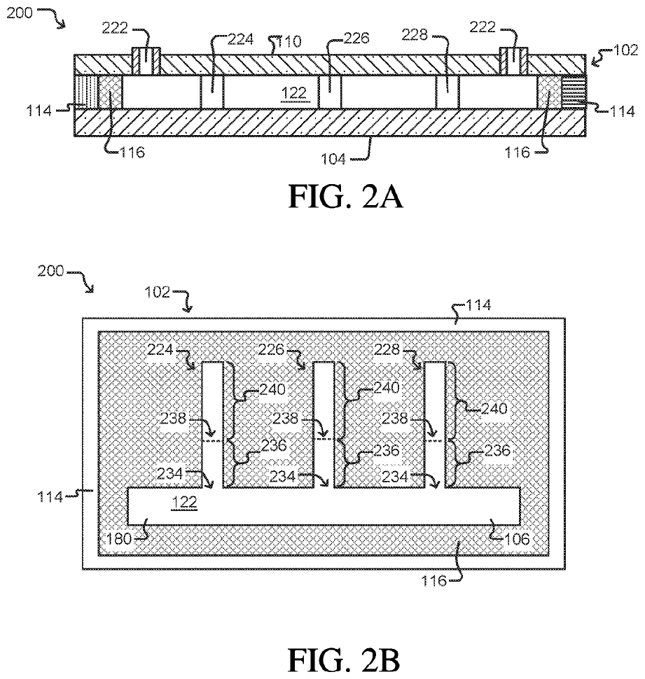

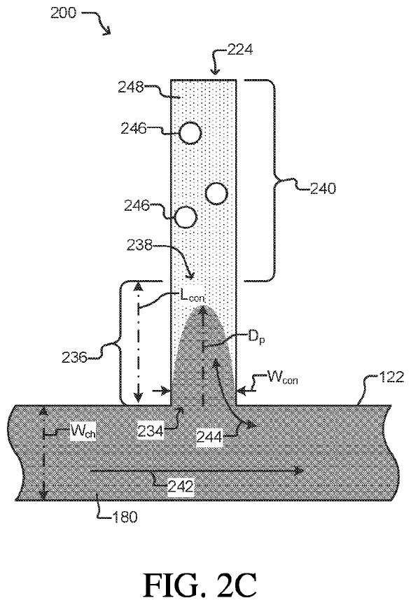

FIGS. 2A-2C show sequestration pens 224, 226, and 228 of a microfluidic device 200, which may be like sequestration pen 128 of FIG. 1A. Each sequestration pen 224, 226, and 228 can comprise an isolation region 240 and a connection region 236 fluidically connecting the isolation region 240 to a flow region, which may, in some embodiments include a microfluidic channel, such as channel 122. The connection region 236 can comprise a proximal opening 234 to the flow region (e.g., microfluidic channel 122) and a distal opening 238 to the isolation region 240. The connection region 236 can be configured so that the maximum penetration depth of a flow of a fluidic medium (not shown) flowing in the microfluidic channel 122 past the sequestration pen 224, 226, and 228 does not extend into the isolation region 240, as discussed below for FIG. 2C. In some embodiments, streamlines from the flow in the microfluidic channel do not enter the isolation region. Thus, due to the connection region 236, a micro-object (not shown) or other material (not shown) disposed in the isolation region 240 of a sequestration pen 224, 226, and 228 can be isolated from, and not substantially affected by, a flow of fluidic medium 180 in the microfluidic channel 122.

The sequestration pens 224, 226, and 228 of FIGS. 2A-2C each have a single opening which opens directly to the microfluidic channel 122. The opening of the sequestration pen may open laterally from the microfluidic channel 122, as shown in FIG. 2A, which depicts a vertical cross-section of microfluidic device 200. FIG. 2B shows a horizontal cross-section of microfluidic device 200. An electrode activation substrate 206 can underlie both the microfluidic channel 122 and the sequestration pens 224, 226, and 228. The upper surface of the electrode activation substrate 206 within an enclosure of a sequestration pen, forming the floor of the sequestration pen, can be disposed at the same level or substantially the same level of the upper surface the of electrode activation substrate 206 within the microfluidic channel 122 (or flow region if a channel is not present), forming the floor of the flow channel (or flow region, respectively) of the microfluidic device. The electrode activation substrate 206 may be featureless or may have an irregular or patterned surface that varies from its highest elevation to its lowest depression by less than about 3 micrometers (microns), 2.5 microns, 2 microns, 1.5 microns, 1 micron, 0.9 microns, 0.5 microns, 0.4 microns, 0.2 microns, 0.1 microns or less. The variation of elevation in the upper surface of the substrate across both the microfluidic channel 122 (or flow region) and sequestration pens may be equal to or less than about 10%, 7%, 5%, 3%, 2%, 1%. 0.9%, 0.8%, 0.5%, 0.3% or 0.1% of the height of the walls of the sequestration pen. Alternatively, the variation of elevation in the upper surface of the substrate across both the microfluidic channel 122 (or flow region) and sequestration pens may be equal to or less than about 2%, 1%. 0.9%, 0.8%, 0.5%, 0.3%, 0.2%, or 0.1% of the height of the substrate. While described in detail for the microfluidic device 200, this may also apply to any of the microfluidic devices described herein.

The microfluidic channel 122 and connection region 236 can be examples of swept regions, and the isolation regions 240 of the sequestration pens 224, 226, and 228 can be examples of unswept regions. Sequestration pens like 224, 226, 228 have isolation regions wherein each isolation region has only one opening, which opens to the connection region of the sequestration pen. Fluidic media exchange in and out of the isolation region so configured can be limited to occurring substantially only by diffusion. As noted, the microfluidic channel 122 and sequestration pens 224, 226, and 228 can be configured to contain one or more fluidic media 180. In the example shown in FIGS. 2A-2B, ports 222 are connected to the microfluidic channel 122 and allow the fluidic medium 180 to be introduced into or removed from the microfluidic device 200. Prior to introduction of the fluidic medium 180, the microfluidic device may be primed with a gas such as carbon dioxide gas. Once the microfluidic device 200 contains the fluidic medium 180, the flow 242 (see FIG. 2C) of fluidic medium 180 in the microfluidic channel 122 can be selectively generated and stopped. For example, as shown, the ports 222 can be disposed at different locations (e.g., opposite ends) of the flow region (microfluidic channel 122), and a flow 242 of the fluidic medium can be created from one port 222 functioning as an inlet to another port 222 functioning as an outlet.

FIG. 2C illustrates a detailed view of an example of a sequestration pen 224, which may contain one or more micro-objects 246, according to some embodiments. The flow 242 of fluidic medium 180 in the microfluidic channel 122 past the proximal opening 234 of the connection region 236 of sequestration pen 224 can cause a secondary flow 244 of the fluidic medium 180 into and out of the sequestration pen 224. To sequester the micro-objects 246 in the isolation region 240 of the sequestration pen 224 from the secondary flow 244, the length L_{con} of the connection region 236 of the sequestration pen 224 (i.e., from the proximal opening 234 to the distal opening 238) should be greater than the penetration depth Dₚ of the secondary flow 244 into the connection region 236. The penetration depth Dₚ depends upon a number of factors, including the shape of the microfluidic channel 122, which may be defined by a width W_{con} of the connection region 236 at the proximal opening 234; a width W_{ch} of the microfluidic channel 122 at the proximal opening 234; a height H_{ch} of the channel 122 at the proximal opening 234; and the width of the distal opening 238 of the connection region 236. Of these factors, the width W_{con} of the connection region 236 at the proximal opening 234 and the height H_{ch} of the channel 122 at the proximal opening 234 tend to be the most significant. In addition, the penetration depth Dₚ can be influenced by the velocity of the fluidic medium 180 in the channel 122 and the viscosity of fluidic medium 180. However, these factors (i.e., velocity and viscosity) can vary widely without dramatic changes in penetration depth Dₚ. For example, for a microfluidic chip 200 having a width W_{con} of the connection region 236 at the proximal opening 234 of about 50 microns, a height H_{ch} of the channel 122 at the proximal opening 122 of about 40 microns, and a width W_{ch} of the microfluidic channel 122 at the proximal opening 122 of about 100 microns to about 150 microns, the penetration depth Dₚ of the secondary flow 244 ranges from less than 1.0 times W_{con} (i.e., less than 50 microns) at a flow rate of 0.1 microliters/sec to about 2.0 times W_{con} (i.e., about 100 microns) at a flow rate of 20 microliters/sec, which represents an increase in Dₚ of only about 2.5-fold over a 200-fold increase in the velocity of the fluidic medium 180.

In some embodiments, the walls of the microfluidic channel 122 and sequestration pen 224, 226, or 228 can be oriented as follows with respect to the vector of the flow 242 of fluidic medium 180 in the microfluidic channel 122: the microfluidic channel width W_{ch} (or cross-sectional area of the microfluidic channel 122) can be substantially perpendicular to the flow 242 of medium 180; the width W_{con} (or cross-sectional area) of the connection region 236 at opening 234 can be substantially parallel to the flow 242 of medium 180 in the microfluidic channel 122; and/or the length L_{con} of the connection region can be substantially perpendicular to the flow 242 of medium 180 in the microfluidic channel 122. The foregoing are examples only, and the relative position of the microfluidic channel 122 and sequestration pens 224, 226 and 228 can be in other orientations with respect to each other.

In some embodiments, for a given microfluidic device, the configurations of the microfluidic channel 122 and the opening 234 may be fixed, whereas the rate of flow 242 of fluidic medium 180 in the microfluidic channel 122 may be variable. Accordingly, for each sequestration pen 224, a maximal velocity Vₘₐₓ for the flow 242 of fluidic medium 180 in channel 122 may be identified that ensures that the penetration depth Dₚ of the secondary flow 244 does not exceed the length L_{con} of the connection region 236. When Vₘₐₓ is not exceeded, the resulting secondary flow 244 can be wholly contained within the connection region 236 and does not enter the isolation region 240. Thus, the flow 242 of fluidic medium 180 in the microfluidic channel 122 (swept region) is prevented from drawing micro-objects 246 out of the isolation region 240, which is an unswept region of the microfluidic circuit, resulting in the micro-objects 246 being retained within the isolation region 240. Accordingly, selection of microfluidic circuit element dimensions and further selection of the operating parameters (e.g., velocity of fluidic medium 180) can prevent contamination of the isolation region 240 of sequestration pen 224 by materials from the microfluidic channel 122 or another sequestration pen 226 or 228. It should be noted, however, that for many microfluidic chip configurations, there is no need to worry about Vₘₐₓ per se, because the chip will break from the pressure associated with flowing fluidic medium 180 at high velocity through the chip before Vₘₐₓ can be achieved.

Components (not shown) in the first fluidic medium 180 in the microfluidic channel 122 can mix with the second fluidic medium 248 in the isolation region 240 substantially only by diffusion of components of the first medium 180 from the microfluidic channel 122 through the connection region 236 and into the second fluidic medium 248 in the isolation region 240. Similarly, components (not shown) of the second medium 248 in the isolation region 240 can mix with the first medium 180 in the microfluidic channel 122 substantially only by diffusion of components of the second medium 248 from the isolation region 240 through the connection region 236 and into the first medium 180 in the microfluidic channel 122.

In some embodiments, the first medium 180 can be the same medium or a different medium than the second medium 248. In some embodiments, the first medium 180 and the second medium 248 can start out being the same, then become different (e.g., through conditioning of the second medium 248 by one or more cells in the isolation region 240, or by changing the medium 180 flowing through the microfluidic channel 122).

As illustrated in FIG. 2C, the width W_{con} of the connection region 236 can be uniform from the proximal opening 234 to the distal opening 238. The width W_{con} of the connection region 236 at the distal opening 238 can be any of the values identified herein for the width W_{con} of the connection region 236 at the proximal opening 234. In some embodiments, the width of the isolation region 240 at the distal opening 238 can be substantially the same as the width W_{con} of the connection region 236 at the proximal opening 234. Alternatively, the width W_{con} of the connection region 236 at the distal opening 238 can be different (e.g., larger or smaller) than the width W_{con} of the connection region 236 at the proximal opening 234. In some embodiments, the width W_{con} of the connection region 236 may be narrowed or widened between the proximal opening 234 and distal opening 238. For example, the connection region 236 may be narrowed or widened between the proximal opening and the distal opening, using a variety of different geometries (e.g., chamfering the connection region, beveling the connection region). Further, any part or subpart of the connection region 236 may be narrowed or widened (e.g., a portion of the connection region adjacent to the proximal opening 234).

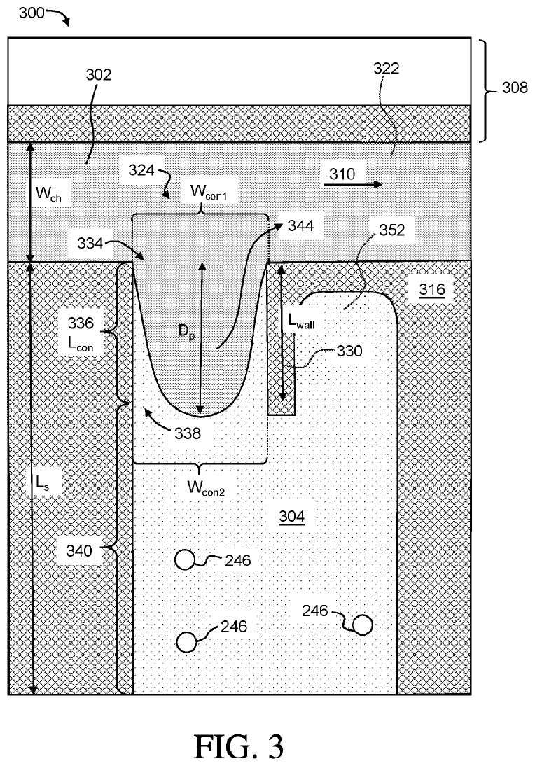

FIG. 3 depicts another exemplary embodiment of a microfluidic device 300 containing microfluidic circuit structure 308, which includes a channel 322 and sequestration pen 324, which has features and properties like any of the sequestration pens described herein for microfluidic devices 100, 175, 200, 400, 520 and any other microfluidic devices described herein.

The exemplary microfluidic devices of FIG. 3 include a microfluidic channel 322, having a width W_{ch}, as described herein, and containing a flow 310 of first fluidic medium 302 and one or more sequestration pens 324 (only one illustrated in FIG. 3). The sequestration pens 324 each have a length Lₛ, a connection region 336, and an isolation region 340, where the isolation region 340 contains a second fluidic medium 304. The connection region 336 has a proximal opening 334, having a width W_{con1}, which opens to the microfluidic channel 322, and a distal opening 338, having a width W_{con2}, which opens to the isolation region 340. The width W_{con1} may or may not be the same as W_{con2}, as described herein. The walls of each sequestration pen 324 may be formed of microfluidic circuit material 316, which may further form the connection region walls 330. A connection region wall 330 can correspond to a structure that is laterally positioned with respect to the proximal opening 334 and at least partially extends into the enclosed portion of the sequestration pen 324. In some embodiments, the length L_{con} of the connection region 336 is at least partially defined by length L_{wall} of the connection region wall 330. The connection region wall 330 may have a length L_{wall}, selected to be more than the penetration depth Dₚ of the secondary flow 344. Thus, the secondary flow 344 can be wholly contained within the connection region without extending into the isolation region 340.

The connection region wall 330 may define a hook region 352, which is a sub-region of the isolation region 340 of the sequestration pen 324. Since the connection region wall 330 extends into the inner cavity of the sequestration pen, the connection region wall 330 can act as a physical barrier to shield hook region 352 from secondary flow 344, with selection of the length of L_{wall}, contributing to the extent of the hook region. In some embodiments, the longer the length L_{wall} of the connection region wall 330, the more sheltered the hook region 352.

In sequestration pens configured like those of FIGS. 2A-2C and 3, the isolation region may have a shape and size of any type, and may be selected to regulate diffusion of nutrients, reagents, and/or media into the sequestration pen to reach to a far wall of the sequestration pen, e.g., opposite the proximal opening of the connection region to the flow region (or microfluidic channel). The size and shape of the isolation region may further be selected to regulate diffusion of waste products and/or secreted products of a biological micro-object out from the isolation region to the flow region via the proximal opening of the connection region of the sequestration pen. In general, the shape of the isolation region is not critical to the ability of the sequestration pen to isolate micro-objects from direct flow in the flow region.

In some other embodiments of sequestration pens, the isolation region may have more than one opening fluidically connecting the isolation region with the flow region of the microfluidic device. However, for an isolation region having a number of n openings fluidically connecting the isolation region to the flow region (or two or more flow regions), n−1 openings can be valved. When the n−1 valved openings are closed, the isolation region has only one effective opening, and exchange of materials into/out of the isolation region occurs only by diffusion.

Examples of microfluidic devices having pens in which biological micro-objects can be placed, cultured, and/or monitored have been described, for example, in U.S. Pat. No. 9,857,333 (Chapman, et al.), U.S. Pat. No. 10,010,882 (White, et al.), and U.S. Pat. No. 9,889,445 (Chapman, et al.), each of which is incorporated herein by reference in its entirety.

Microfluidic circuit element dimensions. Various dimensions and/or features of the sequestration pens and the microfluidic channels to which the sequestration pens open, as described herein, may be selected to limit introduction of contaminants or unwanted micro-objects into the isolation region of a sequestration pen from the flow region/microfluidic channel; limit the exchange of components in the fluidic medium from the channel or from the isolation region to substantially only diffusive exchange; facilitate the transfer of micro-objects into and/or out of the sequestration pens; and/or facilitate growth or expansion of the biological cells. Microfluidic channels and sequestration pens, for any of the embodiments described herein, may have any suitable combination of dimensions, may be selected by one of skill from the teachings of this disclosure.

For any of the microfluidic devices described herein, a microfluidic channel may have a uniform cross sectional height along its length that is a substantially uniform cross sectional height, and may be any cross sectional height as described herein. At any point along the microfluidic channel, the substantially uniform cross sectional height of the channel, the upper surface of which is defined by the inner surface of the cover and the lower surface of which is defined by the inner surface of the base, may be substantially the same as the cross sectional height at any other point along the channel, e.g., having a cross sectional height that is no more than about 10%, about 9%, about 8%, about 7%, about 6%, about 5%, about 4%, about 3%, about 2% or about 1% or less, different from the cross-sectional height of any other location within the channel.

Additionally, the chamber(s), e.g., sequestration pen(s), of the microfluidic devices described herein, may be disposed substantially in a coplanar orientation relative to the microfluidic channel into which the chamber(s) open. That is, the enclosed volume of the chamber(s) is formed by an upper surface that is defined by the inner surface of the cover, a lower surface defined by the inner surface of the base, and walls defined by the microfluidic circuit material. Therefore, the lower surface of the chamber(s) may be coplanar to the lower surface of the microfluidic channel, e.g., substantially coplanar. The upper surface of the chamber may be coplanar to the upper surface of the microfluidic channel, e.g., substantially coplanar. Accordingly, the chamber(s) may have a cross-sectional height, which may have any values as described herein, that is the same as the channel, e.g., substantially the same, and the chamber(s) and microfluidic channel(s) within the microfluidic device may have a substantially uniform cross-sectional height throughout the flow region of the microfluidic device, and may be substantially coplanar throughout the microfluidic device.

Coplanarity of the lower surfaces of the chamber(s) and the microfluidic channel(s) can offer distinct advantage with repositioning micro-objects within the microfluidic device using DEP or magnetic force. Penning and unpenning of micro-objects, and in particular selective penning/selective unpenning, can be greatly facilitated when the lower surfaces of the chamber(s) and the microfluidic channel to which the chamber(s) open have a coplanar orientation.

The proximal opening of the connection region of a sequestration pen may have a width (e.g., W_{con} or W_{con1}) that is at least as large as the largest dimension of a micro-object (e.g., a biological cell, which may be a plant cell, such as a plant protoplast) for which the sequestration pen is intended. In some embodiments, the proximal opening has a width (e.g., W_{con} or W_{con1}) of about 20 microns, about 40 microns, about 50 microns, about 60 microns, about 75 microns, about 100 microns, about 150 microns, about 200 microns, or about 300 microns. The foregoing are examples only, and the width (e.g., W_{con} or W_{con1}) of a proximal opening can be selected to be a value between any of the values listed above (e.g., about 20-200 microns, about 20-150 microns, about 20-100 microns, about 20-75 microns, about 20-60 microns, about 50-300 microns, about 50-200 microns, about 50-150 microns, about 50-100 microns, about 50-75 microns, about 75-150 microns, about 75-100 microns, about 100-300 microns, about 100-200 microns, or about 200-300 microns).

In some embodiments, the connection region of the sequestration pen may have a length (e.g., L_{con}) from the proximal opening to the distal opening to the isolation region of the sequestration pen that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25. times, at least 2.5 times, at least 2.75 times, at least 3.0 times, at least 3.5 times, at least 4.0 times, at least 4.5 times, at least 5.0 times, at least 6.0 times, at least 7.0 times, at least 8.0 times, at least 9.0 times, or at least 10.0 times the width (e.g., W_{con} or W_{con1}) of the proximal opening. Thus, for example, the proximal opening of the connection region of a sequestration pen may have a width (e.g., W_{con} or W_{con1}) from about 20 microns to about 200 microns (e.g., about 50 microns to about 150 microns), and the connection region may have a length L_{con} that is at least 1.0 times (e.g., at least 1.5 times, or at least 2.0 times) the width of the proximal opening. As another example, the proximal opening of the connection region of a sequestration pen may have a width (e.g., W_{con} or W_{con1}) from about 20 microns to about 100 microns (e.g., about 20 microns to about 60 microns), and the connection region may have a length L_{con} that is at least 1.0 times (e.g., at least 1.5 times, or at least 2.0 times) the width of the proximal opening.

The microfluidic channel of a microfluidic device to which a sequestration pen opens may have specified size (e.g., width or height). In some embodiments, the height (e.g., H_{ch}) of the microfluidic channel at a proximal opening to the connection region of a sequestration pen can be within any of the following ranges: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height (e.g., H_{ch}) of the microfluidic channel (e.g., 122) can be selected to be between any of the values listed above. Moreover, the height (e.g., H_{ch}) of the microfluidic channel 122 can be selected to be any of these heights in regions of the microfluidic channel other than at a proximal opening of a sequestration pen.

The width (e.g., W_{ch}) of the microfluidic channel at the proximal opening to the connection region of a sequestration pen can be within any of the following ranges: about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-1000 microns, 50-500 microns, 50-400 microns, 50-300 microns, 50-250 microns, 50-200 microns, 50-150 microns, 50-100 microns, 50-80 microns, 60-300 microns, 60-200 microns, 60-150 microns, 60-100 microns, 60-80 microns, 70-500 microns, 70-400 microns, 70-300 microns, 70-250 microns, 70-200 microns, 70-150 microns, 70-100 microns, 80-100 microns, 90-400 microns, 90-300 microns, 90-250 microns, 90-200 microns, 90-150 microns, 100-300 microns, 100-250 microns, 100-200 microns, 100-150 microns, 100-120 microns, 200-800 microns, 200-700 microns, or 200-600 microns. The foregoing are examples only, and the width (e.g., W_{ch}) of the microfluidic channel can be a value selected to be between any of the values listed above. Moreover, the width (e.g., W_{ch}) of the microfluidic channel can be selected to be in any of these widths in regions of the microfluidic channel other than at a proximal opening of a sequestration pen. In some embodiments, the width W_{ch} of the microfluidic channel at the proximal opening to the connection region of the sequestration pen (e.g., taken transverse to the direction of bulk flow of fluid through the channel) can be substantially perpendicular to a width (e.g., W_{con} or W_{con1}) of the proximal opening.

A cross-sectional area of the microfluidic channel at a proximal opening to the connection region of a sequestration pen can be about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the microfluidic channel at the proximal opening can be selected to be between any of the values listed above. In various embodiments, and the cross-sectional area of the microfluidic channel at regions of the microfluidic channel other than at the proximal opening can also be selected to be between any of the values listed above. In some embodiments, the cross-sectional area is selected to be a substantially uniform value for the entire length of the microfluidic channel.

In some embodiments, the microfluidic chip is configured such that the proximal opening (e.g., 234 or 334) of the connection region of a sequestration pen may have a width (e.g., W_{con} or W_{con1}) from about 20 microns to about 200 microns (e.g., about 50 microns to about 150 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. As another example, the proximal opening (e.g., 234 or 334) of the connection region of a sequestration pen may have a width (e.g., W_{con} or W_{con1}) from about 20 microns to about 100 microns (e.g., about 20 microns to about 60 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. The foregoing are examples only, and the width (e.g., W_{con} or W_{con1}) of the proximal opening (e.g., 234 or 274), the length (e.g., L_{con}) of the connection region, and/or the width (e.g., W_{ch}) of the microfluidic channel (e.g., 122 or 322), can be a value selected to be between any of the values listed above. Generally, however, the width (W_{con} or W_{con1}) of the proximal opening of the connection region of a sequestration pen is less than the width (W_{ch}) of the microfluidic channel. In some embodiments, the width (W_{con} or W_{con1}) of the proximal opening is about 8%, 9%, 10%, 11%, 12%, 13%, 14%, 15%, 16%, 17%, 18%, 19%, 20%, 22%, 24%, 25%, or 30% of the width (W_{ch}) of the microfluidic channel. That is, the width (W_{ch}) of the microfluidic channel may be at least 2.5 times, 3.0 times, 3.5 times, 4.0 times, 4.5 times, 5.0 times, 6.0 times, 7.0 times, 8.0 times, 9.0 times or at least 10.0 times the width (W_{con} or W_{con1}) of the proximal opening of the connection region of the sequestration pen.

In some embodiments, the size W_{C} (e.g., cross-sectional width W_{ch}, diameter, area, or the like) of the channel 122, 322, 618, 718 can be about one and a quarter (1.25), about one and a half (1.5), about two, about two and a half (2.5), about three (3), or more times the size W_{O} (e.g., cross-sectional width W_{con}, diameter, area, or the like) of a chamber opening, e.g., sequestration pen opening 234, 334, and the like. This can reduce the extent of secondary flow and the rate of diffusion (or diffusion flux) through the opening 234, 334 for materials diffusing from a selected chamber (e.g., like sequestration pens 224, 226 of FIG. 2B) into channel 122, 322, 618, 718 and subsequently re-entering a downstream or adjacent chamber (e.g., like sequestration pen 228). The rate of diffusion of a molecule (e.g., a biomolecule of interest, such as an antibody) is dependent on a number of factors, including (without limitation) temperature, viscosity of the medium, and the coefficient of diffusion D₀ of the molecule. For example, the D₀ for an IgG antibody in aqueous solution at about 20° C. is about 4.4×10⁻⁷ cm²/sec, while the kinematic viscosity of cell culture medium is about 9×10−4 m²/sec. Thus, an antibody in cell culture medium at about 20° C. can have a rate of diffusion of about 0.5 microns/sec. Accordingly, in some embodiments, a time period for diffusion from a biological micro-object located within a sequestration pen such as 224, 226, 228, 324 into the channel 122, 322, 618, 718 can be about 10 minutes or less (e.g., about 9, 8, 7, 6, 5 minutes, or less). The time period for diffusion can be manipulated by changing parameters that influence the rate of diffusion. For example, the temperature of the media can be increased (e.g., to a physiological temperature such as about 37° C.) or decreased (e.g., to about 15° C., 10° C., or 4° C.) thereby increasing or decreasing the rate of diffusion, respectively. Alternatively, or in addition, the concentrations of solutes in the medium can be increased or decreased as discussed herein to isolate a selected pen from solutes from other upstream pens.

Accordingly, in some variations, the width (e.g., W_{ch}) of the microfluidic channel at the proximal opening to the connection region of a sequestration pen may be about 50 to 500 microns, about 50 to 300 microns, about 50 to 200 microns, about 70 to 500 microns, about to 70-300 microns, about 70 to 250 microns, about 70 to 200 microns, about 70 to 150 microns, about 70 to 100 microns, about 80 to 500 microns, about 80 to 300 microns, about 80 to 250 microns, about 80 to 200 microns, about 80 to 150 microns, about 90 to 500 microns, about 90 to 300 microns, about 90 to 250 microns, about 90 to 200 microns, about 90 to 150 microns, about 100 to 500 microns, about 100 to 300 microns, about 100 to 250 microns, about 100 to 200 microns, or about 100 to 150 microns. In some embodiments, the width W_{ch} of the microfluidic channel at the proximal opening to the connection region of a sequestration pen may be about 70 to 250 microns, about 80 to 200 microns, or about 90 to 150 microns. The width W_{con} of the opening of the chamber (e.g., sequestration pen) may be about 20 to 100 microns; about 30 to 90 microns; or about 20 to 60 microns. In some embodiments, W_{ch} is about 70-250 microns and W_{con} is about 20 to 100 microns; W_{ch} is about 80 to 200 microns and W_{con} is about 30 to 90 microns; W_{ch} is about 90 to 150 microns, and W_{con} is about 20 to 60 microns; or any combination of the widths of W_{ch} and W_{con} thereof.

In some embodiments, the proximal opening (e.g., 234 or 334) of the connection region of a sequestration pen has a width (e.g., W_{con} or W_{con1}) that is 2.0 times or less (e.g., 2.0, 1.9, 1.8, 1.5, 1.3, 1.0, 0.8, 0.5, or 0.1 times) the height (e.g., H_{ch}) of the flow region/microfluidic channel at the proximal opening, or has a value that lies within a range defined by any two of the foregoing values.

In some embodiments, the width W_{con1} of a proximal opening (e.g., 234 or 334) of a connection region of a sequestration pen may be the same as a width W_{con2} of the distal opening (e.g., 238 or 338) to the isolation region thereof. In some embodiments, the width W_{con1} of the proximal opening may be different than a width W_{con2} of the distal opening, and W_{con1} and/or W_{con2} may be selected from any of the values described for W_{con} or W_{con1}. In some embodiments, the walls (including a connection region wall) that define the proximal opening and distal opening may be substantially parallel with respect to each other. In some embodiments, the walls that define the proximal opening and distal opening may be selected to not be parallel with respect to each other.

The length (e.g., L_{con}) of the connection region can be about 1-600 microns, 5-550 microns, 10-500 microns, 15-400 microns, 20-300 microns, 20-500 microns, 40-400 microns, 60-300 microns, 80-200 microns, about 100-150 microns, about 20-300 microns, about 20-250 microns, about 20-200 microns, about 20-150 microns, about 20-100 microns, about 30-250 microns, about 30-200 microns, about 30-150 microns, about 30-100 microns, about 30-80 microns, about 30-50 microns, about 45-250 microns, about 45-200 microns, about 45-100 microns, about 45-80 microns, about 45-60 microns, about 60-200 microns, about 60-150 microns, about 60-100 microns or about 60-80 microns. The foregoing are examples only, and length (e.g., L_{con}) of a connection region can be selected to be a value that is between any of the values listed above.

The connection region wall of a sequestration pen may have a length (e.g., L_{wall}) that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, or at least 3.5 times the width (e.g., W_{con} or W_{con1}) of the proximal opening of the connection region of the sequestration pen. In some embodiments, the connection region wall may have a length L_{wall} of about 20-200 microns, about 20-150 microns, about 20-100 microns, about 20-80 microns, or about 20-50 microns. The foregoing are examples only, and a connection region wall may have a length L_{wall} selected to be between any of the values listed above.

A sequestration pen may have a length Lₛ of about 40-600 microns, about 40-500 microns, about 40-400 microns, about 40-300 microns, about 40-200 microns, about 40-100 microns or about 40-80 microns. The foregoing are examples only, and a sequestration pen may have a length Lₛ selected to be between any of the values listed above.

According to some embodiments, a sequestration pen may have a specified height (e.g., Hₛ). In some embodiments, a sequestration pen has a height Hₛ of about 20 microns to about 200 microns (e.g., about 20 microns to about 150 microns, about 20 microns to about 100 microns, about 20 microns to about 60 microns, about 30 microns to about 150 microns, about 30 microns to about 100 microns, about 30 microns to about 60 microns, about 40 microns to about 150 microns, about 40 microns to about 100 microns, or about 40 microns to about 60 microns). The foregoing are examples only, and a sequestration pen can have a height Hₛ selected to be between any of the values listed above.

The height H_{con} of a connection region at a proximal opening of a sequestration pen can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{con} of the connection region can be selected to be between any of the values listed above. Typically, the height H_{con} of the connection region is selected to be the same as the height H_{ch} of the microfluidic channel at the proximal opening of the connection region. Additionally, the height Hₛ of the sequestration pen is typically selected to be the same as the height H_{con} of a connection region and/or the height H_{ch} of the microfluidic channel. In some embodiments, Hₛ, H_{con}, and H_{ch} may be selected to be the same value of any of the values listed above for a selected microfluidic device.

The isolation region can be configured to contain only one, two, three, four, five, or a similar relatively small number of micro-objects. In other embodiments, the isolation region may contain more than 10, more than 50 or more than 100 micro-objects. Accordingly, the volume of an isolation region can be, for example, at least 1×10⁴, 1×10⁵, 5×10⁵, 8×10⁵, 1×10⁶, 2×10⁶, 4×10⁶, 6×10⁶, 1×10⁷, 3×10⁷, 5×10⁷ 1×10⁸, 5×10⁸, or 8×10⁸ cubic microns, or more. The foregoing are examples only, and the isolation region can be configured to contain numbers of micro-objects and volumes selected to be between any of the values listed above (e.g., a volume between 1×10⁵ cubic microns and 5×10⁵ cubic microns, between 5×10⁵ cubic microns and 1×10⁶ cubic microns, between 1×10⁶ cubic microns and 2×10⁶ cubic microns, or between 2×10⁶ cubic microns and 1×10⁷ cubic microns).

According to some embodiments, a sequestration pen of a microfluidic device may have a specified volume. The specified volume of the sequestration pen (or the isolation region of the sequestration pen) may be selected such that a single cell or a small number of cells (e.g., 2-10 or 2-5) can rapidly condition the medium and thereby attain favorable (or optimal) growth conditions. In some embodiments, the sequestration pen has a volume of about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more. In some embodiments, the sequestration pen has a volume of about 1 nanoliter to about 50 nanoliters, 2 nanoliters to about 25 nanoliters, 2 nanoliters to about 20 nanoliters, about 2 nanoliters to about 15 nanoliters, or about 2 nanoliters to about 10 nanoliters. The foregoing are examples only, and a sequestration pen can have a volume selected to be any value that is between any of the values listed above.

According to some embodiments, the flow of fluidic medium within the microfluidic channel (e.g., 122 or 322) may have a specified maximum velocity (e.g., Vₘₐₓ). In some embodiments, the maximum velocity (e.g., Vₘₐₓ) may be set at around 0.2, 0.5, 0.7, 1.0, 1.3, 1.5, 2.0, 2.5, 3.0, 3.5, 4.0, 4.5, 5.0, 5.5, 6.0, 6.7, 7.0, 7.5, 8.0, 8.5, 9.0, 10, 11, 12, 13, 14, 15, 16, 17, 18, 19, 20, 22, 24, or 25 microliters/sec. The foregoing are examples only, and the flow of fluidic medium within the microfluidic channel can have a maximum velocity (e.g., Vₘₐₓ) selected to be a value between any of the values listed above. The flow of fluidic medium within the microfluidic channel typically may be flowed at a rate less than the Vₘₐₓ. While the Vₘₐₓ may vary depending on the specific size and numbers of channel and sequestration pens opening thereto, a fluidic medium may be flowed at about 0.1 microliters/sec to about 20 microliters/sec; about 0.1 microliters/sec to about 15 microliters/sec; about 0.1 microliters/sec to about 12 microliters/sec, about 0.1 microliters/sec to about 10 microliters/sec; about 0.1 microliter/sec to about 7 microliters/sec without exceeding the Vₘₐₓ. In some portions of a typical workflow, a flow rate of a fluidic medium may be about 0.1 microliters/sec; about 0.5 microliters/sec; about 1.0 microliters/sec; about 2.0 microliters/sec; about 3.0 microliters/sec; about 4.0 microliters/sec; about 5.0 microliters/sec; about 6.0 microliters/sec; about 7.0 microliters/sec; about 8.0 microliters/sec; about 9.0 microliters/sec; about 10.0 microliters/sec; about 11.0 microliters/sec; or any range defined by two of the foregoing values, e.g., 1-5 microliters/sec or 5-10 microliters/sec. The flow rate of a fluidic medium in the microfluidic channel may be equal to or less than about 12 microliters/sec; about 10 microliters/sec; about 8 microliters/sec, or about 6 microliters/sec.

In various embodiment, the microfluidic device has sequestration pens configured as in any of the embodiments discussed herein where the microfluidic device has about 5 to about 10 sequestration pens, about 10 to about 50 sequestration pens, about 25 to about 200 sequestration pens, about 100 to about 500 sequestration pens, about 200 to about 1000 sequestration pens, about 500 to about 1500 sequestration pens, about 1000 to about 2500 sequestration pens, about 2000 to about 5000 sequestration pens, about 3500 to about 7000 sequestration pens, about 5000 to about 10,000 sequestration pens, about 7,500 to about 15,000 sequestration pens, about 12,500 to about 20,000 sequestration pens, about 15,000 to about 25,000 sequestration pens, about 20,000 to about 30,000 sequestration pens, about 25,000 to about 35,000 sequestration pens, about 30,000 to about 40,000 sequestration pens, about 35,000 to about 45,000 sequestration pens, or about 40,000 to about 50,000 sequestration pens. The sequestration pens need not all be the same size and may include a variety of configurations (e.g., different widths, different features within the sequestration pen).

Coating solutions and coating agents. In some embodiments, at least one inner surface of the microfluidic device includes a coating material that provides a layer of organic and/or hydrophilic molecules suitable for maintenance, expansion and/or movement of biological micro-object(s) (i.e., the biological micro-object exhibits increased viability, greater expansion and/or greater portability within the microfluidic device). The conditioned surface may reduce surface fouling, participate in providing a layer of hydration, and/or otherwise shield the biological micro-objects from contact with the non-organic materials of the microfluidic device interior.

In some embodiments, substantially all the inner surfaces of the microfluidic device include the coating material. The coated inner surface(s) may include the surface of a flow region (e.g., channel), chamber, or sequestration pen, or a combination thereof. In some embodiments, each of a plurality of sequestration pens has at least one inner surface coated with coating materials. In other embodiments, each of a plurality of flow regions or channels has at least one inner surface coated with coating materials. In some embodiments, at least one inner surface of each of a plurality of sequestration pens and each of a plurality of channels is coated with coating materials. The coating may be applied before or after introduction of biological micro-object(s), or may be introduced concurrently with the biological micro-object(s). In some embodiments, the biological micro-object(s) may be imported into the microfluidic device in a fluidic medium that includes one or more coating agents. In other embodiments, the inner surface(s) of the microfluidic device (e.g., a microfluidic device having an electrode activation substrate such as, but not limited to, a device including dielectrophoresis (DEP) electrodes) may be treated or “primed” with a coating solution comprising a coating agent prior to introduction of the biological micro-object(s) into the microfluidic device. Any convenient coating agent/coating solution can be used, including but not limited to: serum or serum factors, bovine serum albumin (BSA), polymers, detergents, enzymes, and any combination thereof.

Synthetic polymer-based coating materials. The at least one inner surface may include a coating material that comprises a polymer. The polymer may be non-covalently bound (e.g., it may be non-specifically adhered) to the at least one surface. The polymer may have a variety of structural motifs, such as found in block polymers (and copolymers), star polymers (star copolymers), and graft or comb polymers (graft copolymers), all of which may be suitable for the methods disclosed herein. A wide variety of alkylene ether containing polymers may be suitable for use in the microfluidic devices described herein, including but not limited to Pluronic® polymers such as Pluronic® L44, L64, P85, and F127 (including F127NF). Other examples of suitable coating materials are described in US2016/0312165, the contents of which are herein incorporated by reference in their entirety.

Covalently linked coating materials. In some embodiments, the at least one inner surface includes covalently linked molecules that provide a layer of organic and/or hydrophilic molecules suitable for maintenance/expansion of biological micro-object(s) within the microfluidic device, providing a conditioned surface for such cells. The covalently linked molecules include a linking group, wherein the linking group is covalently linked to one or more surfaces of the microfluidic device, as described below. The linking group is also covalently linked to a surface modifying moiety configured to provide a layer of organic and/or hydrophilic molecules suitable for maintenance/expansion/movement of biological micro-object(s).

In some embodiments, the covalently linked moiety configured to provide a layer of organic and/or hydrophilic molecules suitable for maintenance/expansion of biological micro-object(s) may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); amino groups (including derivatives thereof, such as, but not limited to alkylated amines, hydroxyalkylated amino group, guanidinium, and heterocylic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaines; sulfamic acids; or amino acids.

In various embodiments, the covalently linked moiety configured to provide a layer of organic and/or hydrophilic molecules suitable for maintenance/expansion of biological micro-object(s) in the microfluidic device may include non-polymeric moieties such as an alkyl moiety, amino acid moiety, alcohol moiety, amino moiety, carboxylic acid moiety, phosphonic acid moiety, sulfonic acid moiety, sulfamic acid moiety, or saccharide moiety. Alternatively, the covalently linked moiety may include polymeric moieties, which may include any of these moieties.

In some embodiments, a microfluidic device may have a hydrophobic layer upon the inner surface of the base which includes a covalently linked alkyl moiety. The covalently linked alkyl moiety may comprise carbon atoms forming a linear chain (e.g., a linear chain of at least 10 carbons, or at least 14, 16, 18, 20, 22, or more carbons) and may be an unbranched alkyl moiety. In some embodiments, the alkyl group may include a substituted alkyl group (e.g., some of the carbons in the alkyl group can be fluorinated or perfluorinated). In some embodiments, the alkyl group may include a first segment, which may include a perfluoroalkyl group, joined to a second segment, which may include a non-substituted alkyl group, where the first and second segments may be joined directly or indirectly (e.g., by means of an ether linkage). The first segment of the alkyl group may be located distal to the linking group, and the second segment of the alkyl group may be located proximal to the linking group.

In other embodiments, the covalently linked moiety may include at least one amino acid, which may include more than one type of amino acid. Thus, the covalently linked moiety may include a peptide or a protein. In some embodiments, the covalently linked moiety may include an amino acid which may provide a zwitterionic surface to support cell growth, viability, portability, or any combination thereof.

In other embodiments, the covalently linked moiety may further include a streptavidin or biotin moiety. In some embodiments, a modified biological moiety such as, for example, a biotinylated protein or peptide may be introduced to the inner surface of a microfluidic device bearing covalently linked streptavidin, and couple via the covalently linked streptavidin to the surface, thereby providing a modified surface presenting the protein or peptide.

In other embodiments, the covalently linked moiety may include at least one alkylene oxide moiety and may include any alkylene oxide polymer as described above. One useful class of alkylene ether containing polymers is polyethylene glycol (PEG M_{w}<100,000 Da) or alternatively polyethylene oxide (PEO, M_{w}>100,000). In some embodiments, a PEG may have an M_{w} of about 1000 Da, 5000 Da, 10,000 Da or 20,000 Da. In some embodiments, the PEG polymer may further be substituted with a hydrophilic or charged moiety, such as but not limited to an alcohol functionality or a carboxylic acid moiety.

The covalently linked moiety may include one or more saccharides. The covalently linked saccharides may be mono-, di-, or polysaccharides. The covalently linked saccharides may be modified to introduce a reactive pairing moiety which permits coupling or elaboration for attachment to the surface. One exemplary covalently linked moiety may include a dextran polysaccharide, which may be coupled indirectly to a surface via an unbranched linker.

The coating material providing a conditioned surface may comprise only one kind of covalently linked moiety or may include more than one different kind of covalently linked moiety. For example, a polyethylene glycol conditioned surface may have covalently linked alkylene oxide moieties having a specified number of alkylene oxide units which are all the same, e.g., having the same linking group and covalent attachment to the surface, the same overall length, and the same number of alkylene oxide units. Alternatively, the coating material may have more than one kind of covalently linked moiety attached to the surface. For example, the coating material may include the molecules having covalently linked alkylene oxide moieties having a first specified number of alkylene oxide units and may further include a further set of molecules having bulky moieties such as a protein or peptide connected to a covalently attached alkylene oxide linking moiety having a greater number of alkylene oxide units. The different types of molecules may be varied in any suitable ratio to obtain the surface characteristics desired. For example, the conditioned surface having a mixture of first molecules having a chemical structure having a first specified number of alkylene oxide units and second molecules including peptide or protein moieties, which may be coupled via a biotin/streptavidin binding pair to the covalently attached alkylene linking moiety, may have a ratio of first molecules:second molecules of about 99:1; about 90:10; about 75:25; about 50:50; about 30:70; about 20:80; about 10:90; or any ratio selected to be between these values. In this instance, the first set of molecules having different, less sterically demanding termini and fewer backbone atoms can help to functionalize the entire substrate surface and thereby prevent undesired adhesion or contact with the silicon/silicon oxide, hafnium oxide or alumina making up the substrate itself. The selection of the ratio of mixture of first molecules to second molecules may also modulate the surface modification introduced by the second molecules bearing peptide or protein moieties.

Conditioned surface properties. Various factors can alter the physical thickness of the conditioned surface, such as the manner in which the conditioned surface is formed on the substrate (e.g., vapor deposition, liquid phase deposition, spin coating, flooding, and electrostatic coating). In some embodiments, the conditioned surface may have a thickness of about 1 nm to about 10 nm. In some embodiments, the covalently linked moieties of the conditioned surface may form a monolayer when covalently linked to the surface of the microfluidic device (which may include an electrode activation substrate having dielectrophoresis (DEP) or electrowetting (EW) electrodes) and may have a thickness of less than 10 nm (e.g., less than 5 nm, or about 1.5 to 3.0 nm). These values are in contrast to that of a surface prepared by spin coating, for example, which may typically have a thickness of about 30 nm. In some embodiments, the conditioned surface does not require a perfectly formed monolayer to be suitably functional for operation within a DEP-configured microfluidic device. In other embodiments, the conditioned surface formed by the covalently linked moieties may have a thickness of about 10 nm to about 50 nm.

Unitary or Multi-part conditioned surface. The covalently linked coating material may be formed by reaction of a molecule which already contains the moiety configured to provide a layer of organic and/or hydrophilic molecules suitable for maintenance/expansion of biological micro-object(s) in the microfluidic device, and may have a structure of Formula I, as shown below. Alternatively, the covalently linked coating material may be formed in a two-part sequence, having a structure of Formula II, by coupling the moiety configured to provide a layer of organic and/or hydrophilic molecules suitable for maintenance and/or expansion of biological micro-object(s) to a surface modifying ligand that itself has been covalently linked to the surface. In some embodiments, the surface may be formed in a two-part or three-part sequence, including a streptavidin/biotin binding pair, to introduce a protein, peptide, or mixed modified surface.

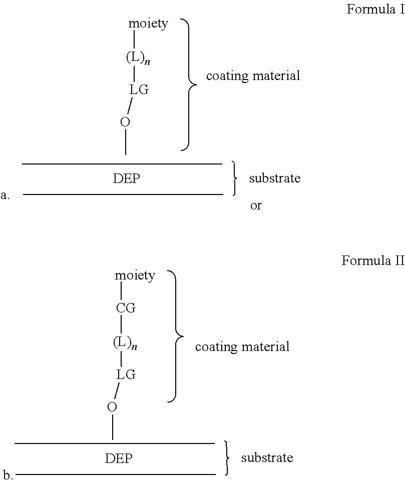

The coating material may be linked covalently to oxides of the surface of a DEP-configured or EW-configured substrate. The coating material may be attached to the oxides via a linking group (“LG”), which may be a siloxy or phosphonate ester group formed from the reaction of a siloxane or phosphonic acid group with the oxides. The moiety configured to provide a layer of organic and/or hydrophilic molecules suitable for maintenance/expansion of biological micro-object(s) in the microfluidic device can be any of the moieties described herein. The linking group LG may be directly or indirectly connected to the moiety configured to provide a layer of organic and/or hydrophilic molecules suitable for maintenance/expansion of biological micro-object(s) in the microfluidic device. When the linking group LG is directly connected to the moiety, optional linker (“L”) is not present and n is 0. When the linking group LG is indirectly connected to the moiety, linker L is present and n is 1. The linker L may have a linear portion where a backbone of the linear portion may include 1 to 200 non-hydrogen atoms selected from any combination of silicon, carbon, nitrogen, oxygen, sulfur and/or phosphorus atoms, subject to chemical bonding limitations as is known in the art. It may be interrupted with any combination of one or more moieties, which may be chosen from ether, amino, carbonyl, amido, and/or phosphonate groups, arylene, heteroarylene, or heterocyclic groups. In some embodiments, the coupling group CG represents the resultant group from reaction of a reactive moiety Rₓ and a reactive pairing moiety Rₚₓ (i.e., a moiety configured to react with the reactive moiety Rₓ). CG may be a carboxamidyl group, a triazolylene group, substituted triazolylene group, a carboxamidyl, thioamidyl, an oxime, a mercaptyl, a disulfide, an ether, or alkenyl group, or any other suitable group that may be formed upon reaction of a reactive moiety with its respective reactive pairing moiety. In some embodiments, CG may further represent a streptavidin/biotin binding pair.

Further details of suitable coating treatments and modifications, as well as methods of preparation, may be found at U.S. Patent Application Publication No. US2016/0312165 (Lowe, Jr., et al.), U.S. Patent Application Publication No US2017/0173580 (Lowe, Jr., et al), International Patent Application Publication WO2017/205830 (Lowe, Jr., et al.), and International Patent Application Publication WO2019/01880 (Beemiller et al.), each of which disclosures is herein incorporated by reference in its entirety.

Microfluidic device motive technologies. The microfluidic devices described herein can be used with any type of motive technology. As described herein, the control and monitoring equipment of the system can comprise a motive module for selecting and moving objects, such as micro-objects or droplets, in the microfluidic circuit of a microfluidic device. The motive technology(ies) may include, for example, dielectrophoresis (DEP), electrowetting (EW), and/or other motive technologies. The microfluidic device can have a variety of motive configurations, depending upon the type of object being moved and other considerations. Returning to FIG. 1A, for example, the support structure 104 and/or cover 110 of the microfluidic device 100 can comprise DEP electrode activation substrates for selectively inducing motive forces on micro-objects in the fluidic medium 180 in the microfluidic circuit 120 and thereby select, capture, and/or move individual micro-objects or groups of micro-objects.

In some embodiments, motive forces are applied across the fluidic medium 180 (e.g., in the flow path and/or in the sequestration pens) via one or more electrodes (not shown) to manipulate, transport, separate and sort micro-objects located therein. For example, in some embodiments, motive forces are applied to one or more portions of microfluidic circuit 120 in order to transfer a single micro-object from the flow path 106 into a desired microfluidic sequestration pen. In some embodiments, motive forces are used to prevent a micro-object within a sequestration pen from being displaced therefrom. Further, in some embodiments, motive forces are used to selectively remove a micro-object from a sequestration pen that was previously collected in accordance with the embodiments of the current disclosure.

In some embodiments, the microfluidic device is configured as an optically-actuated electrokinetic device, such as in optoelectronic tweezer (OET) and/or optoelectrowetting (OEW) configured device. Examples of suitable OET configured devices (e.g., containing optically actuated dielectrophoresis electrode activation substrates) can include those illustrated in U.S. Pat. No. RE 44,711 (Wu, et al.) (originally issued as U.S. Pat. No. 7,612,355), U.S. Pat. No. 7,956,339 (Ohta, et al.), U.S. Pat. No. 9,908,115 (Hobbs et al.), and U.S. Pat. No. 9,403,172 (Short et al), each of which is incorporated herein by reference in its entirety. Examples of suitable OEW configured devices can include those illustrated in U.S. Pat. No. 6,958,132 (Chiou, et al.), and U.S. Pat. No. 9,533,306 (Chiou, et al.), each of which is incorporated herein by reference in its entirety. Examples of suitable optically-actuated electrokinetic devices that include combined OET/OEW configured devices can include those illustrated in U.S. Patent Application Publication No. 2015/0306598 (Khandros, et al.), U.S. Patent Application Publication No 2015/0306599 (Khandros, et al.), and U.S. Patent Application Publication No. 2017/0173580 (Lowe, et al.), each of which is incorporated herein by reference in its entirety.

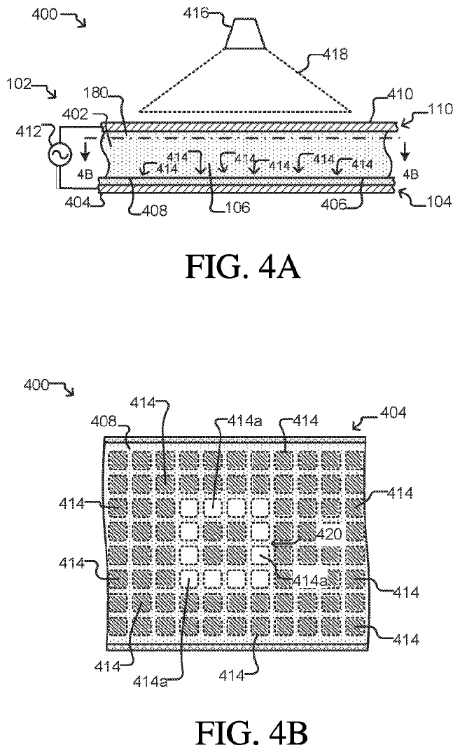

It should be understood that, for purposes of simplicity, the various examples of FIGS. 1-5B may illustrate portions of microfluidic devices while not depicting other portions. Further, FIGS. 1-5B may be part of, and implemented as, one or more microfluidic systems. In one non-limiting example, FIGS. 4A and 4B show a side cross-sectional view and a top cross-sectional view, respectively, of a portion of an enclosure 102 of the microfluidic device 400 having a region/chamber 402, which may be part of a fluidic circuit element having a more detailed structure, such as a growth chamber, a sequestration pen (which may be like any sequestration pen described herein), a flow region, or a flow channel. For instance, microfluidic device 400 may be similar to microfluidic devices 100, 175, 200, 300, 520 or any other microfluidic device as described herein. Furthermore, the microfluidic device 400 may include other fluidic circuit elements and may be part of a system including control and monitoring equipment 152, described above, having one or more of the media module 160, motive module 162, imaging module 164, optional tilting module 166, and other modules 168. Microfluidic devices 175, 200, 300, 520 and any other microfluidic devices described herein may similarly have any of the features described in detail for FIGS. 1A-1B and 4A-4B.

As shown in the example of FIG. 4A, the microfluidic device 400 includes a support structure 104 having a bottom electrode 404 and an electrode activation substrate 406 overlying the bottom electrode 404, and a cover 110 having a top electrode 410, with the top electrode 410 spaced apart from the bottom electrode 404. The top electrode 410 and the electrode activation substrate 406 define opposing surfaces of the region/chamber 402. A fluidic medium 180 contained in the region/chamber 402 thus provides a resistive connection between the top electrode 410 and the electrode activation substrate 406. A power source 412 configured to be connected to the bottom electrode 404 and the top electrode 410 and create a biasing voltage between the electrodes, as required for the generation of DEP forces in the region/chamber 402, is also shown. The power source 412 can be, for example, an alternating current (AC) power source.

In certain embodiments, the microfluidic device 200 illustrated in FIGS. 4A and 4B can have an optically-actuated DEP electrode activation substrate. Accordingly, changing patterns of light 418 from the light source 416, which may be controlled by the motive module 162, can selectively activate and deactivate changing patterns of DEP electrodes at regions 414 of the inner surface 408 of the electrode activation substrate 406. (Hereinafter the regions 414 of a microfluidic device having a DEP electrode activation substrate are referred to as “DEP electrode regions.”) As illustrated in FIG. 4B, a light pattern 418 directed onto the inner surface 408 of the electrode activation substrate 406 can illuminate select DEP electrode regions 414a (shown in white) in a pattern, such as a square. The non-illuminated DEP electrode regions 414 (cross-hatched) are hereinafter referred to as “dark” DEP electrode regions 414. The relative electrical impedance through the DEP electrode activation substrate 406 (i.e., from the bottom electrode 404 up to the inner surface 408 of the electrode activation substrate 406 which interfaces with the fluidic medium 180 in the flow region 106) is greater than the relative electrical impedance through the fluidic medium 180 in the region/chamber 402 (i.e., from the inner surface 408 of the electrode activation substrate 406 to the top electrode 410 of the cover 110) at each dark DEP electrode region 414. An illuminated DEP electrode region 414a, however, exhibits a reduced relative impedance through the electrode activation substrate 406 that is less than the relative impedance through the fluidic medium 180 in the region/chamber 402 at each illuminated DEP electrode region 414a.

With the power source 412 activated, the foregoing DEP configuration creates an electric field gradient in the fluidic medium 180 between illuminated DEP electrode regions 414a and adjacent dark DEP electrode regions 414, which in turn creates local DEP forces that attract or repel nearby micro-objects (not shown) in the fluidic medium 180. DEP electrodes that attract or repel micro-objects in the fluidic medium 180 can thus be selectively activated and deactivated at many different such DEP electrode regions 414 at the inner surface 408 of the region/chamber 402 by changing light patterns 418 projected from a light source 416 into the microfluidic device 400. Whether the DEP forces attract or repel nearby micro-objects can depend on such parameters as the frequency of the power source 412 and the dielectric properties of the fluidic medium 180 and/or micro-objects (not shown). Depending on the frequency of the power applied to the DEP configuration and selection of fluidic media (e.g., a highly conductive media such as PBS or other media appropriate for maintaining biological cells), negative DEP forces may be produced. Negative DEP forces may repel the micro-objects away from the location of the induced non-uniform electrical field. In some embodiments, a microfluidic device incorporating DEP technology may generate negative DEP forces.

The square pattern 420 of illuminated DEP electrode regions 414a illustrated in FIG. 4B is an example only. Any pattern of the DEP electrode regions 414 can be illuminated (and thereby activated) by the pattern of light 418 projected into the microfluidic device 400, and the pattern of illuminated/activated DEP electrode regions 414 can be repeatedly changed by changing or moving the light pattern 418.

In some embodiments, the electrode activation substrate 406 can comprise or consist of a photoconductive material. In such embodiments, the inner surface 408 of the electrode activation substrate 406 can be featureless. For example, the electrode activation substrate 406 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 m. In such embodiments, the DEP electrode regions 414 can be created anywhere and in any pattern on the inner surface 408 of the electrode activation substrate 406, in accordance with the light pattern 418. The number and pattern of the DEP electrode regions 214 thus need not be fixed, but can correspond to the light pattern 418. Examples of microfluidic devices having a DEP configuration comprising a photoconductive layer such as discussed above have been described, for example, in U.S. Pat. No. RE 44,711 (Wu, et al.) (originally issued as U.S. Pat. No. 7,612,355), each of which is incorporated herein by reference in its entirety.

In other embodiments, the electrode activation substrate 406 can comprise a substrate comprising a plurality of doped layers, electrically insulating layers (or regions), and electrically conductive layers that form semiconductor integrated circuits, such as is known in semiconductor fields. For example, the electrode activation substrate 406 can comprise a plurality of phototransistors, including, for example, lateral bipolar phototransistors, with each phototransistor corresponding to a DEP electrode region 414. Alternatively, the electrode activation substrate 406 can comprise electrodes (e.g., conductive metal electrodes) controlled by phototransistor switches, with each such electrode corresponding to a DEP electrode region 414. The electrode activation substrate 406 can include a pattern of such phototransistors or phototransistor-controlled electrodes. The pattern, for example, can be an array of substantially square phototransistors or phototransistor-controlled electrodes arranged in rows and columns. Alternatively, the pattern can be an array of substantially hexagonal phototransistors or phototransistor-controlled electrodes that form a hexagonal lattice. Regardless of the pattern, electric circuit elements can form electrical connections between the DEP electrode regions 414 at the inner surface 408 of the electrode activation substrate 406 and the bottom electrode 404, and those electrical connections (i.e., phototransistors or electrodes) can be selectively activated and deactivated by the light pattern 418, as described above.

Examples of microfluidic devices having electrode activation substrates that comprise phototransistors have been described, for example, in U.S. Pat. No. 7,956,339 (Ohta et al.) and U.S. Pat. No. 9,908,115 (Hobbs et al.), the entire contents of each of which are incorporated herein by reference. Examples of microfluidic devices having electrode activation substrates that comprise electrodes controlled by phototransistor switches have been described, for example, in U.S. Pat. No. 9,403,172 (Short et al.), which is incorporated herein by reference in its entirety.

In some embodiments of a DEP configured microfluidic device, the top electrode 410 is part of a first wall (or cover 110) of the enclosure 102, and the electrode activation substrate 406 and bottom electrode 404 are part of a second wall (or support structure 104) of the enclosure 102. The region/chamber 402 can be between the first wall and the second wall. In other embodiments, the electrode 410 is part of the second wall (or support structure 104) and one or both of the electrode activation substrate 406 and/or the electrode 410 are part of the first wall (or cover 110). Moreover, the light source 416 can alternatively be used to illuminate the enclosure 102 from below.

With the microfluidic device 400 of FIGS. 4A-4B having a DEP electrode activation substrate, the motive module 162 of control and monitoring equipment 152, as described for FIG. 1A herein, can select a micro-object (not shown) in the fluidic medium 180 in the region/chamber 402 by projecting a light pattern 418 into the microfluidic device 400 to activate a first set of one or more DEP electrodes at DEP electrode regions 414a of the inner surface 408 of the electrode activation substrate 406 in a pattern (e.g., square pattern 420) that surrounds and captures the micro-object. The motive module 162 can then move the in situ-generated captured micro-object by moving the light pattern 418 relative to the microfluidic device 400 to activate a second set of one or more DEP electrodes at DEP electrode regions 414. Alternatively, the microfluidic device 400 can be moved relative to the light pattern 418.

In other embodiments, the microfluidic device 400 may be a DEP configured device that does not rely upon light activation of DEP electrodes at the inner surface 408 of the electrode activation substrate 406. For example, the electrode activation substrate 406 can comprise selectively addressable and energizable electrodes positioned opposite to a surface including at least one electrode (e.g., cover 110). Switches (e.g., transistor switches in a semiconductor substrate) may be selectively opened and closed to activate or inactivate DEP electrodes at DEP electrode regions 414, thereby creating a net DEP force on a micro-object (not shown) in region/chamber 402 in the vicinity of the activated DEP electrodes. Depending on such characteristics as the frequency of the power source 412 and the dielectric properties of the medium (not shown) and/or micro-objects in the region/chamber 402, the DEP force can attract or repel a nearby micro-object. By selectively activating and deactivating a set of DEP electrodes (e.g., at a set of DEP electrodes regions 414 that forms a square pattern 420), one or more micro-objects in region/chamber 402 can be selected and moved within the region/chamber 402. The motive module 162 in FIG. 1A can control such switches and thus activate and deactivate individual ones of the DEP electrodes to select, and move particular micro-objects (not shown) around the region/chamber 402. Microfluidic devices having a DEP electrode activation substrate that includes selectively addressable and energizable electrodes are known in the art and have been described, for example, in U.S. Pat. No. 6,294,063 (Becker, et al.) and U.S. Pat. No. 6,942,776 (Medoro), each of which is incorporated herein by reference in its entirety.

Regardless of whether the microfluidic device 400 has a dielectrophoretic electrode activation substrate, an electrowetting electrode activation substrate or a combination of both a dielectrophoretic and an electrowetting activation substrate, a power source 412 can be used to provide a potential (e.g., an AC voltage potential) that powers the electrical circuits of the microfluidic device 400. The power source 412 can be the same as, or a component of, the power source 192 referenced in FIG. 1A. Power source 412 can be configured to provide an AC voltage and/or current to the top electrode 410 and the bottom electrode 404. For an AC voltage, the power source 412 can provide a frequency range and an average or peak power (e.g., voltage or current) range sufficient to generate net DEP forces (or electrowetting forces) strong enough to select and move individual micro-objects (not shown) in the region/chamber 402, as discussed above, and/or to change the wetting properties of the inner surface 408 of the support structure 104 in the region/chamber 402, as also discussed above. Such frequency ranges and average or peak power ranges are known in the art. See, e.g., U.S. Pat. No. 6,958,132 (Chiou, et al.), U.S. Pat. No. RE44,711 (Wu, et al.) (originally issued as U.S. Pat. No. 7,612,355), and U.S. Patent Application Publication Nos. 2014/0124370 (Short, et al.), 2015/0306598 (Khandros, et al.), 2015/0306599 (Khandros, et al.), and 2017/0173580 (Lowe, Jr. et al.), each of which disclosures are herein incorporated by reference in its entirety.

Other forces may be utilized within the microfluidic devices, alone or in combination, to move selected micro-objects. Bulk fluidic flow within the microfluidic channel may move micro-objects within the flow region. Localized fluidic flow, which may be operated within the microfluidic channel, within a sequestration pen, or within another kind of chamber (e.g., a reservoir) can also be used to move selected micro-objects. Localized fluidic flow can be used to move selected micro-objects out of the flow region into a non-flow region such as a sequestration pen or the reverse, from a non-flow region into a flow region. The localized flow can be actuated by deforming a deformable wall of the microfluidic device, as described in U.S. Pat. No. 10,058,865 (Breinlinger, et al.), which is incorporated herein by reference in its entirety.

Gravity may be used to move micro-objects within the microfluidic channel, into a sequestration pen, and/or out of a sequestration pen or other chamber, as described in U.S. Pat. No. 9,744,533 (Breinlinger, et al.), which is incorporated herein by reference in its entirety. Use of gravity (e.g., by tilting the microfluidic device and/or the support to which the microfluidic device is attached) may be useful for bulk movement of cells into or out of the sequestration pens from/to the flow region. Magnetic forces may be employed to move micro-objects including paramagnetic materials, which can include magnetic micro-objects attached to or associated with a biological micro-object. Alternatively, or in additional, centripetal forces may be used to move micro-objects within the microfluidic channel, as well as into or out of sequestration pens or other chambers in the microfluidic device.

In another alternative mode of moving micro-objects, laser-generated dislodging forces may be used to export micro-objects or assist in exporting micro-objects from a sequestration pen or any other chamber in the microfluidic device, as described in International Patent Publication No. WO2017/117408 (Kurz, et al.), which is incorporated herein by reference in its entirety.

In some embodiments, DEP forces are combined with other forces, such as fluidic flow (e.g., bulk fluidic flow in a channel or localized fluidic flow actuated by deformation of a deformable surface of the microfluidic device, laser generated dislodging forces, and/or gravitational force), so as to manipulate, transport, separate and sort micro-objects and/or droplets within the microfluidic circuit 120. In some embodiments, the DEP forces can be applied prior to the other forces. In other embodiments, the DEP forces can be applied after the other forces. In still other instances, the DEP forces can be applied in an alternating manner with the other forces. For the microfluidic devices described herein, repositioning of micro-objects may not generally rely upon gravity or hydrodynamic forces to position or trap micro-objects at a selected position. Gravity may be chosen as one form of repositioning force, but the ability to reposition of micro-objects within the microfluidic device does not rely solely upon the use of gravity. While fluid flow in the microfluidic channels may be used to introduce micro-objects into the microfluidic channels (e.g., flow region), such regional flow is not relied upon to pen or unpen micro-objects, while localized flow (e.g., force derived from actuating a deformable surface) may, in some embodiments, be selected from amongst the other types of repositioning forces described herein to pen or unpen micro-objects or to export them from the microfluidic device.

When DEP is used to reposition micro-objects, bulk fluidic flow in a channel is generally stopped prior to applying DEP to micro-objects to reposition the micro-objects within the microfluidic circuit of the device, whether the micro-objects are being repositioned from the channel into a sequestration pen or from a sequestration pen into the channel. Bulk fluidic flow may be resumed thereafter.

System. Returning to FIG. 1A, a system 150 for operating and controlling microfluidic devices is shown, such as for controlling the microfluidic device 100. The electrical power source 192 can provide electric power to the microfluidic device 100, providing biasing voltages or currents as needed. The electrical power source 192 can, for example, comprise one or more alternating current (AC) and/or direct current (DC) voltage or current sources.

System 150 can further include a media source 178. The media source 178 (e.g., a container, reservoir, or the like) can comprise multiple sections or containers, each for holding a different fluidic medium 180. Thus, the media source 178 can be a device that is outside of and separate from the microfluidic device 100, as illustrated in FIG. 1A. Alternatively, the media source 178 can be located in whole or in part inside the enclosure 102 of the microfluidic device 100. For example, the media source 178 can comprise reservoirs that are part of the microfluidic device 100.

FIG. 1A also illustrates simplified block diagram depictions of examples of control and monitoring equipment 152 that constitute part of system 150 and can be utilized in conjunction with a microfluidic device 100. As shown, examples of such control and monitoring equipment 152 can include a master controller 154 comprising a media module 160 for controlling the media source 178, a motive module 162 for controlling movement and/or selection of micro-objects (not shown) and/or medium (e.g., droplets of medium) in the microfluidic circuit 120, an imaging module 164 for controlling an imaging device (e.g., a camera, microscope, light source or any combination thereof) for capturing images (e.g., digital images), and an optional tilting module 166 for controlling the tilting of the microfluidic device 100. The control equipment 152 can also include other modules 168 for controlling, monitoring, or performing other functions with respect to the microfluidic device 100. As shown, the monitoring equipment 152 can further include a display device 170 and an input/output device 172.

The master controller 154 can comprise a control module 156 and a digital memory 158. The control module 156 can comprise, for example, a digital processor configured to operate in accordance with machine executable instructions (e.g., software, firmware, source code, or the like) stored as non-transitory data or signals in the memory 158. Alternatively, or in addition, the control module 156 can comprise hardwired digital circuitry and/or analog circuitry. The media module 160, motive module 162, imaging module 164, optional tilting module 166, and/or other modules 168 can be similarly configured. Thus, functions, processes acts, actions, or steps of a process discussed herein as being performed with respect to the microfluidic device 100 or any other microfluidic apparatus can be performed by any one or more of the master controller 154, media module 160, motive module 162, imaging module 164, optional tilting module 166, and/or other modules 168 configured as discussed above. Similarly, the master controller 154, media module 160, motive module 162, imaging module 164, optional tilting module 166, and/or other modules 168 may be communicatively coupled to transmit and receive data used in any function, process, act, action or step discussed herein.

The media module 160 controls the media source 178. For example, the media module 160 can control the media source 178 to input a selected fluidic medium 180 into the enclosure 102 (e.g., through an inlet port 107). The media module 160 can also control removal of media from the enclosure 102 (e.g., through an outlet port (not shown)). One or more media can thus be selectively input into and removed from the microfluidic circuit 120. The media module 160 can also control the flow of fluidic medium 180 in the flow path 106 inside the microfluidic circuit 120. The media module 160 may also provide conditioning gaseous conditions to the media source 178, for example, providing an environment containing 5% CO₂ (or higher). The media module 160 may also control the temperature of an enclosure of the media source, for example, to provide feeder cells in the media source with proper temperature control.

Motive module. The motive module 162 can be configured to control selection and movement of micro-objects (not shown) in the microfluidic circuit 120. The enclosure 102 of the microfluidic device 100 can comprise one or more electrokinetic mechanisms including a dielectrophoresis (DEP) electrode activation substrate, optoelectronic tweezers (OET) electrode activation substrate, electrowetting (EW) electrode activation substrate, and/or an opto-electrowetting (OEW) electrode activation substrate, where the motive module 162 can control the activation of electrodes and/or transistors (e.g., phototransistors) to select and move micro-objects and/or droplets in the flow path 106 and/or within sequestration pens 124, 126, 128, and 130. The electrokinetic mechanism(s) may be any suitable single or combined mechanism as described within the paragraphs describing motive technologies for use within the microfluidic device. A DEP configured device may include one or more electrodes that apply a non-uniform electric field in the microfluidic circuit 120 sufficient to exert a dielectrophoretic force on micro-objects in the microfluidic circuit 120. An OET configured device may include photo-activatable electrodes to provide selective control of movement of micro-objects in the microfluidic circuit 120 via light-induced dielectrophoresis.

The imaging module 164 can control the imaging device. For example, the imaging module 164 can receive and process image data from the imaging device. Image data from the imaging device can comprise any type of information captured by the imaging device (e.g., the presence or absence of micro-objects, droplets of medium, accumulation of label, such as fluorescence label, etc.). Using the information captured by the imaging device, the imaging module 164 can further calculate the position of objects (e.g., micro-objects, droplets of medium) and/or the rate of motion of such objects within the microfluidic device 100.

The imaging device (part of imaging module 164, discussed below) can comprise a device, such as a digital camera, for capturing images inside microfluidic circuit 120. In some instances, the imaging device further comprises a detector having a fast frame rate and/or high sensitivity (e.g., for low light applications). The imaging device can also include a mechanism for directing stimulating radiation and/or light beams into the microfluidic circuit 120 and collecting radiation and/or light beams reflected or emitted from the microfluidic circuit 120 (or micro-objects contained therein). The emitted light beams may be in the visible spectrum and may, e.g., include fluorescence emissions. The reflected light beams may include reflected emissions originating from an LED or a wide spectrum lamp, such as a mercury lamp (e.g., a high-pressure mercury lamp) or a Xenon arc lamp. The imaging device may further include a microscope (or an optical train), which may or may not include an eyepiece.

Support Structure. System 150 may further comprise a support structure 190 configured to support and/or hold the enclosure 102 comprising the microfluidic circuit 120. In some embodiments, the optional tilting module 166 can be configured to activate the support structure 190 to rotate the microfluidic device 100 about one or more axes of rotation. The optional tilting module 166 can be configured to support and/or hold the microfluidic device 100 in a level orientation (i.e., at 0° relative to x- and y-axes), a vertical orientation (i.e., at 900 relative to the x-axis and/or the y-axis), or any orientation therebetween. The orientation of the microfluidic device 100 (and the microfluidic circuit 120) relative to an axis is referred to herein as the “tilt” of the microfluidic device 100 (and the microfluidic circuit 120). For example, support structure 190 can optionally be used to tilt the microfluidic device 100 (e.g., as controlled by optional tilting module 166) to 0.1°, 0.2°, 0.3°, 0.4°, 0.5°, 0.6°, 0.7°, 0.8°, 0.9°, 1°, 2°, 3°, 4°, 5°, 10°, 15°, 20°, 25°, 30°, 35°, 40°, 45°, 50°, 55°, 60°, 650, 70°, 75°, 80°, 90° relative to the x-axis or any degree therebetween. When the microfluidic device is tilted at angles greater than about 15, tilting may be performed to create bulk movement of micro-objects into/out of sequestration pens from/into the flow region (e.g., microfluidic channel). In some embodiments, the support structure 190 can hold the microfluidic device 100 at a fixed angle of 0.1°, 0.2°, 0.3°, 0.4°, 0.5°, 0.6°, 0.7°, 0.8°, 0.9°, 1°, 2°, 3°, 4°, 5°, or 10° relative to the x-axis (horizontal), so long as DEP is an effective force to move micro-objects out of the sequestration pens into the microfluidic channel. Since the surface of the electrode activation substrate is substantially flat, DEP forces may be used even when the far end of the sequestration pen, opposite its opening to the microfluidic channel, is disposed at a position lower in a vertical direction than the microfluidic channel.

In some embodiments where the microfluidic device is tilted or held at a fixed angle relative to horizontal, the microfluidic device 100 may be disposed in an orientation such that the inner surface of the base of the flow path 106 is positioned at an angle above or below the inner surface of the base of the one or more sequestration pens opening laterally to the flow path. The term “above” as used herein denotes that the flow path 106 is positioned higher than the one or more sequestration pens on a vertical axis defined by the force of gravity (i.e., an object in a sequestration pen above a flow path 106 would have a higher gravitational potential energy than an object in the flow path), and inversely, for positioning of the flow path 106 below one or more sequestration pens. In some embodiments, the support structure 190 may be held at a fixed angle of less than about 5°, about 4°, about 3° or less than about 2° relative to the x-axis (horizontal), thereby placing the sequestration pens at a lower potential energy relative to the flow path. In some other embodiments, when long term culturing (e.g., for more than about 2, 3, 4, 5, 6, 7 or more days) is performed within the microfluidic device, the device may be supported on a culturing support and may be tilted at a greater angle of about 10°, 15°, 20°, 25°, 30°, or any angle therebetween to retain biological micro-objects within the sequestration pens during the long-term culturing period. At the end of the culturing period, the microfluidic device containing the cultured biological micro-objects may be returned to the support 190 within system 150, where the angle of tilting is decreased to values as described above, affording the use of DEP to move the biological micro-objects out of the sequestration pens. Further examples of the use of gravitational forces induced by tilting are described in U.S. Pat. No. 9,744,533 (Breinlinger et al.), the contents of which are herein incorporated by reference in its entirety.

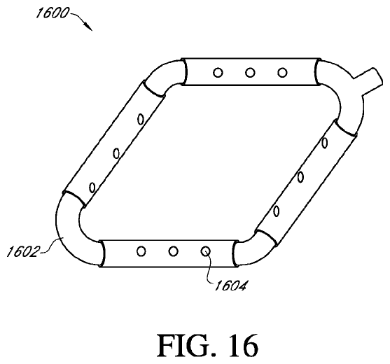

In some embodiments, the support structure 190 is further configured to support and/or hold an oxygen delivery module, such as an oxygen delivery module described herein with respect to FIG. 16. In some embodiments, the support structure is configured to support and/or hold the oxygen delivery module in proximity to the microfluidic device. In some embodiments, the support structure is configured to support and/or hold the oxygen delivery module such that the oxygen delivery module surrounds the microfluidic device. In some embodiments, the support structure is configured to support and/or hold the oxygen delivery module a distance of at least about 1 millimeter (mm), 2 mm, 3 mm, 4 mm, 5 mm, 6 mm, 7 mm, 8 mm, 9 mm, 10 mm, or more from the microfluidic device. In some embodiments, the support structure is configured to support and/or hold the oxygen delivery module a distance of at most about 10 mm, 9 mm, 8 mm, 7 mm, 6 mm, 5 mm, 4 mm, 3 mm, 2 mm, 1 mm, or less from the microfluidic device. In some embodiments, the support structure is configured to support and/or hold the oxygen delivery module a distance from the microfluidic device that ranges between any two of the preceding values.

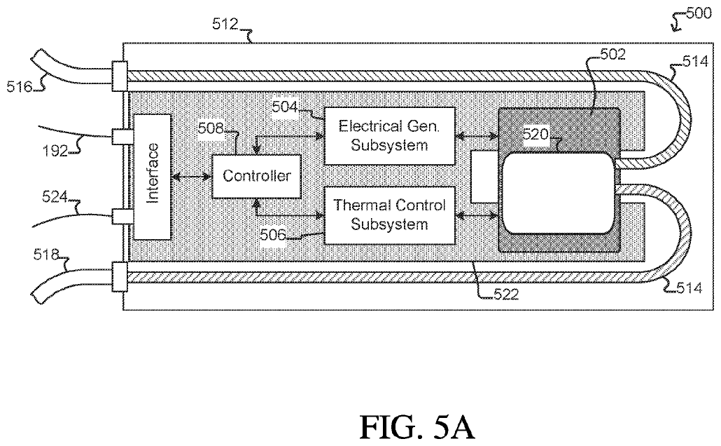

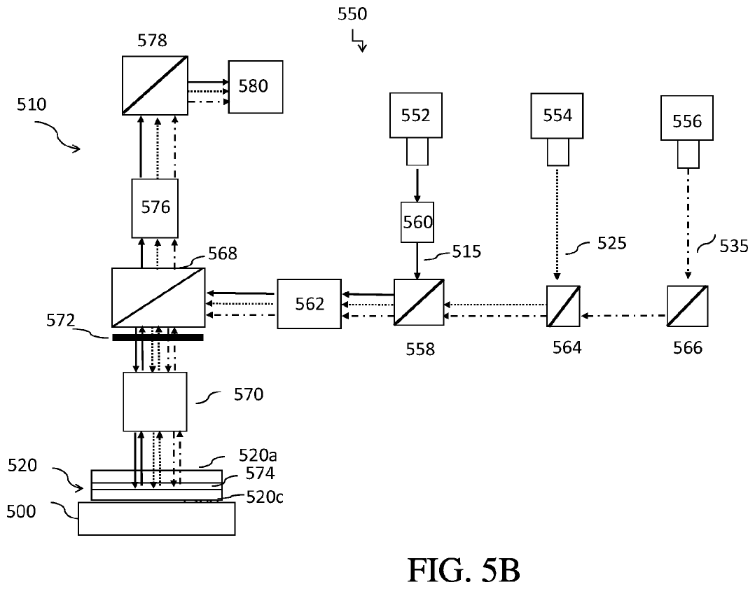

Nest. Turning now to FIG. 5A, the system 150 can include a structure (also referred to as a “nest”) 500 configured to hold a microfluidic device 520, which may be like microfluidic device 100, 200, or any other microfluidic device described herein. The nest 500 can include a socket 502 capable of interfacing with the microfluidic device 520 (e.g., an optically actuated electrokinetic device 100, 200, etc.) and providing electrical connections from power source 192 to microfluidic device 520. The nest 500 can further include an integrated electrical signal generation subsystem 504. The electrical signal generation subsystem 504 can be configured to supply a biasing voltage to socket 502 such that the biasing voltage is applied across a pair of electrodes in the microfluidic device 520 when it is being held by socket 502. Thus, the electrical signal generation subsystem 504 can be part of power source 192. The ability to apply a biasing voltage to microfluidic device 520 does not mean that a biasing voltage will be applied at all times when the microfluidic device 520 is held by the socket 502. Rather, in most cases, the biasing voltage will be applied intermittently, e.g., only as needed to facilitate the generation of electrokinetic forces, such as dielectrophoresis or electro-wetting, in the microfluidic device 520.

As illustrated in FIG. 5A, the nest 500 can include a printed circuit board assembly (PCBA) 522. The electrical signal generation subsystem 504 can be mounted on and electrically integrated into the PCBA 522. The exemplary support includes socket 502 mounted on PCBA 522, as well.

In some embodiments, the nest 500 can comprise an electrical signal generation subsystem 504 configured to measure the amplified voltage at the microfluidic device 520 and then adjust its own output voltage as needed such that the measured voltage at the microfluidic device 520 is the desired value. In some embodiments, the waveform amplification circuit can have a +6.5V to −6.5V power supply generated by a pair of DC-DC converters mounted on the PCBA 322, resulting in a signal of up to 13 Vpp at the microfluidic device 520.

In certain embodiments, the nest 500 further comprises a controller 508, such as a microprocessor used to sense and/or control the electrical signal generation subsystem 504. Examples of suitable microprocessors include the Arduino™ microprocessors, such as the Arduino Nano™. The controller 508 may be used to perform functions and analysis or may communicate with an external master controller 154 (shown in FIG. 1A) to perform functions and analysis. In the embodiment illustrated in FIG. 3A the controller 308 communicates with the master controller 154 (of FIG. 1A) through an interface (e.g., a plug or connector).

As illustrated in FIG. 5A, the support structure 500 (e.g., nest) can further include a thermal control subsystem 506. The thermal control subsystem 506 can be configured to regulate the temperature of microfluidic device 520 held by the support structure 500. For example, the thermal control subsystem 506 can include a Peltier thermoelectric device (not shown) and a cooling unit (not shown). In the embodiment illustrated in FIG. 5A, the support structure 500 comprises an inlet 516 and an outlet 518 to receive cooled fluid from an external reservoir (not shown) of the cooling unit, introduce the cooled fluid into the fluidic path 514 and through the cooling block, and then return the cooled fluid to the external reservoir. In some embodiments, the Peltier thermoelectric device, the cooling unit, and/or the fluidic path 514 can be mounted on a casing 512 of the support structure 500. In some embodiments, the thermal control subsystem 506 is configured to regulate the temperature of the Peltier thermoelectric device so as to achieve a target temperature for the microfluidic device 520. Temperature regulation of the Peltier thermoelectric device can be achieved, for example, by a thermoelectric power supply, such as a Pololu™ thermoelectric power supply (Pololu Robotics and Electronics Corp.). The thermal control subsystem 506 can include a feedback circuit, such as a temperature value provided by an analog circuit. Alternatively, the feedback circuit can be provided by a digital circuit.

The nest 500 can include a serial port 524 which allows the microprocessor of the controller 508 to communicate with an external master controller 154 via the interface. In addition, the microprocessor of the controller 508 can communicate (e.g., via a Plink tool (not shown)) with the electrical signal generation subsystem 504 and thermal control subsystem 506. Thus, via the combination of the controller 508, the interface, and the serial port 524, the electrical signal generation subsystem 504 and the thermal control subsystem 506 can communicate with the external master controller 154. In this manner, the master controller 154 can, among other things, assist the electrical signal generation subsystem 504 by performing scaling calculations for output voltage adjustments. A Graphical User Interface (GUI) (not shown) provided via a display device 170 coupled to the external master controller 154, can be configured to plot temperature and waveform data obtained from the thermal control subsystem 506 and the electrical signal generation subsystem 504, respectively. Alternatively, or in addition, the GUI can allow for updates to the controller 508, the thermal control subsystem 506, and the electrical signal generation subsystem 504.

Optical sub-system. FIG. 5B is a schematic of an optical sub-system 550 having an optical apparatus 510 for imaging and manipulating micro-objects in a microfluidic device 520, which can be any microfluidic device described herein. The optical apparatus 510 can be configured to perform imaging, analysis and manipulation of one or more micro-objects within the enclosure of the microfluidic device 520.

The optical apparatus 510 may have a first light source 552, a second light source 554, and a third light source 556. The first light source 552 can transmit light to a structured light modulator 560, which can include a digital mirror device (DMD) or a microshutter array system (MSA), either of which can be configured to receive light from the first light source 552 and selectively transmit a subset of the received light into the optical apparatus 510. Alternatively, the structured light modulator 560 can include a device that produces its own light (and thus dispenses with the need for a light source 552), such as an organic light emitting diode display (OLED), a liquid crystal on silicon (LCOS) device, a ferroelectric liquid crystal on silicon device (FLCOS), or a transmissive liquid crystal display (LCD). The structured light modulator 560 can be, for example, a projector. Thus, the structured light modulator 560 can be capable of emitting both structured and unstructured light. In certain embodiments, an imaging module and/or motive module of the system can control the structured light modulator 560.

In embodiments when the structured light modulator 560 includes a mirror, the modulator can have a plurality of mirrors. Each mirror of the plurality of mirrors can have a size of about 5 microns×5 microns to about 10 microns×10 microns, or any values therebetween. The structured light modulator 560 can include an array of mirrors (or pixels) that is 2000×1000, 2580×1600, 3000×2000, or any values therebetween. In some embodiments, only a portion of an illumination area of the structured light modulator 560 is used. The structured light modulator 560 can transmit the selected subset of light to a first dichroic beam splitter 558, which can reflect this light to a first tube lens 562.

The first tube lens 562 can have a large clear aperture, for example, a diameter larger than about 40 mm to about 50 mm, or more, providing a large field of view. Thus, the first tube lens 562 can have an aperture that is large enough to capture all (or substantially all) of the light beams emanating from the structured light modulator 560.

The structured light 515 having a wavelength of about 400 nm to about 710 nm, may alternatively or in addition, provide fluorescence excitation illumination to the microfluidic device.

The second light source 554 may provide unstructured brightfield illumination. The brightfield illumination light 525 may have any suitable wavelength, and in some embodiments, may have a wavelength of about 400 nm to about 760 nm. The second light source 554 can transmit light to a second dichroic beam splitter 564 (which also may receive illumination light 535 from the third light source 556), and the second light, brightfield illumination light 525, may be transmitted therefrom to the first dichroic beam splitter 558. The second light, brightfield illumination light 525, may then be transmitted from the first dichroic beam splitter 558 to the first tube lens 562.

The third light source 556 can transmit light through a matched pair relay lens (not shown) to a mirror 566. The third illumination light 535 may therefrom be reflected to the second dichroic beam splitter 5338 and be transmitted therefrom to the first beam splitter 5338, and onward to the first tube lens 5381. The third illumination light 535 may be a laser and may have any suitable wavelength. In some embodiments, the laser illumination 535 may have a wavelength of about 350 nm to about 900 nm. The laser illumination 535 may be configured to heat portions of one or more sequestration pens within the microfluidic device. The laser illumination 535 may be configured to heat fluidic medium, a micro-object, a wall or a portion of a wall of a sequestration pen, a metal target disposed within a microfluidic channel or sequestration pen of the microfluidic channel, or a photoreversible physical barrier within the microfluidic device, and described in more detail in U. S. Application Publication Nos. 2017/0165667 (Beaumont, et al.) and 2018/0298318 (Kurz, et al.), each of which disclosure is herein incorporated by reference in its entirety. In other embodiments, the laser illumination 535 may be configured to initiate photocleavage of surface modifying moieties of a modified surface of the microfluidic device or photocleavage of moieties providing adherent functionalities for micro-objects within a sequestration pen within the microfluidic device. Further details of photocleavage using a laser may be found in International Application Publication No. WO2017/205830 (Lowe, Jr. et al.), which disclosure is herein incorporated by reference in its entirety.

The light from the first, second, and third light sources (552, 554, 556) passes through the first tube lens 562 and is transmitted to a third dichroic beam splitter 568 and filter changer 572. The third dichroic beam splitter 568 can reflect a portion of the light and transmit the light through one or more filters in the filter changer 572 and to the objective 570, which may be an objective changer with a plurality of different objectives that can be switched on demand. Some of the light (515, 525, and/or 535) may pass through the third dichroic beam splitter 568 and be terminated or absorbed by a beam block (not shown). The light reflected from the third dichroic beam splitter 568 passes through the objective 570 to illuminate the sample plane 574, which can be a portion of a microfluidic device 520 such as the sequestration pens described herein.

The nest 500, as described in FIG. 5A, can be integrated with the optical apparatus 510 and be a part of the apparatus 510. The nest 500 can provide electrical connection to the enclosure and be further configured to provide fluidic connections to the enclosure. Users may load the microfluidic apparatus 520 into the nest 500. In some other embodiments, the nest 500 can be a separate component independent of the optical apparatus 510.

Light can be reflected off and/or emitted from the sample plane 574 to pass back through the objective 570, through the filter changer 572, and through the third dichroic beam splitter 568 to a second tube lens 576. The light can pass through the second tube lens 576 (or imaging tube lens 576) and be reflected from a mirror 578 to an imaging sensor 580. Stray light baffles (not shown) can be placed between the first tube lens 562 and the third dichroic beam splitter 568, between the third dichroic beam splitter 568 and the second tube lens 576, and between the second tube lens 576 and the imaging sensor 580.

Objective. The optical apparatus can comprise the objective lens 570 that is specifically designed and configured for viewing and manipulating of micro-objects in the microfluidic device 520. For example, conventional microscope objective lenses are designed to view micro-objects on a slide or through 5 mm of aqueous fluid, while micro-objects in the microfluidic device 520 are inside the plurality of sequestration pens within the viewing plane 574 which have a depth of 20, 30, 40, 50, 60 70, 80 microns or any values therebetween. In some embodiments, a transparent cover 520a, for example, glass or ITO cover with a thickness of about 750 microns, can be placed on top of the plurality of sequestration pens, which are disposed above a microfluidic substrate 520c. Thus, the images of the micro-objects obtained by using the conventional microscope objective lenses may have large aberrations such as spherical and chromatic aberrations, which can degrade the quality of the images. The objective lens 570 of the optical apparatus 510 can be configured to correct the spherical and chromatic aberrations in the optical apparatus 1350. The objective lens 570 can have one or more magnification levels available such as, 4×, 10×, 20×.

Modes of illumination. In some embodiments, the structured light modulator 560 can be configured to modulate light beams received from the first light source 552 and transmits a plurality of illumination light beams 515, which are structured light beams, into the enclosure of the microfluidic device, e.g., the region containing the sequestration pens. The structured light beams can comprise the plurality of illumination light beams. The plurality of illumination light beams can be selectively activated to generate a plurality of illuminations patterns. In some embodiments, the structured light modulator 560 can be configured to generate an illumination pattern, similarly as described for FIGS. 4A-4B, which can be moved and adjusted. The optical apparatus 560 can further comprise a control unit (not shown) which is configured to adjust the illumination pattern to selectively activate the one or more of the plurality of DEP electrodes of a substrate 520c and generate DEP forces to move the one or more micro-objects inside the plurality of sequestration pens within the microfluidic device 520. For example, the plurality of illuminations patterns can be adjusted over time in a controlled manner to manipulate the micro-objects in the microfluidic device 520. Each of the plurality of illumination patterns can be shifted to shift the location of the DEP force generated and to move the structured light for one position to another in order to move the micro-objects within the enclosure of the microfluidic apparatus 520.

In some embodiments, the optical apparatus 510 may be configured such that each of the plurality of sequestration pens in the sample plane 574 within the field of view is simultaneously in focus at the image sensor 580 and at the structured light modulator 560. In some embodiments, the structured light modulator 560 can be disposed at a conjugate plane of the image sensor 580. In various embodiments, the optical apparatus 510 can have a confocal configuration or confocal property. The optical apparatus 510 can be further configured such that only each interior area of the flow region and/or each of the plurality of sequestration pens in the sample plane 574 within the field of view is imaged onto the image sensor 580 in order to reduce overall noise to thereby increase the contrast and resolution of the image.

In some embodiments, the first tube lens 562 can be configured to generate collimated light beams and transmit the collimated light beams to the objective lens 570. The objective 570 can receive the collimated light beams from the first tube lens 562 and focus the collimated light beams into each interior area of the flow region and each of the plurality of sequestration pens in the sample plane 574 within the field of view of the image sensor 580 or the optical apparatus 510. In some embodiments, the first tube lens 562 can be configured to generate a plurality of collimated light beams and transmit the plurality of collimated light beams to the objective lens 570. The objective 570 can receive the plurality of collimated light beams from the first tube lens 562 and converge the plurality of collimated light beams into each of the plurality of sequestration pens in the sample plane 574 within the field of view of the image sensor 580 or the optical apparatus 510.

In some embodiments, the optical apparatus 510 can be configured to illuminate the at least a portion of sequestration pens with a plurality of illumination spots. The objective 570 can receive the plurality of collimated light beams from the first tube lens 562 and project the plurality of illumination spots, which may form an illumination pattern, into each of the plurality of sequestration pens in the sample plane 574 within the field of view. For example, each of the plurality of illumination spots can have a size of about 5 microns×5 microns; 10 microns×10 microns; 10 microns×30 microns, 30 microns×60 microns, 40 microns×40 microns, 40 microns×60 microns, 60 microns×120 microns, 80 microns×100 microns, 100 microns×140 microns and any values there between. The illumination spots may individually have a shape that is circular, square, or rectangular. Alternatively, the illumination spots may be grouped within a plurality of illumination spots (e.g., an illumination pattern) to form a larger polygonal shape such as a rectangle, square, or wedge shape. The illumination pattern may enclose (e.g., surround) an unilluminated space that may be square, rectangular or polygonal. For example, each of the plurality of illumination spots can have an area of about 150 to about 3000, about 4000 to about 10000, or 5000 to about 15000 square microns. An illumination pattern may have an area of about 1000 to about 8000, about 4000 to about 10000, 7000 to about 20000, 8000 to about 22000, 10000 to about 25000 square microns and any values there between.

The optical system 510 may be used to determine how to reposition micro-objects and into and out of the sequestration pens of the microfluidic device, as well as to count the number of micro-objects present within the microfluidic circuit of the device. Further details of repositioning and counting micro-objects are found in U. S. Application Publication No. 2016/0160259 (Du); U.S. Pat. No. 9,996,920 (Du et al.); and International Application Publication No. WO2017/102748 (Kim, et al.). The optical system 510 may also be employed in assay methods to determine concentrations of reagents/assay products, and further details are found in U.S. Pat. No. 8,921,055 (Chapman), U.S. Pat. No. 10,010,882 (White et al.), and U.S. Pat. No. 9,889,445 (Chapman et al.); International Application Publication No. WO2017/181135 (Lionberger, et al.); and International Application Serial No. PCT/US2018/055918 (Lionberger, et al.). Further details of the features of optical apparatuses suitable for use within a system for observing and manipulating micro-objects within a microfluidic device, as described herein, may be found in WO2018/102747 (Lundquist, et al), the disclosure of which is herein incorporated by reference in its entirety.

Additional system components for maintenance of viability of cells within a chamber of a microfluidic device. In order to promote growth and/or expansion of cell populations, environmental conditions conducive to maintaining healthy, functional cells may be provided by additional components of the system. For example, such additional components can provide nutrients, cell growth signaling species, pH modulation, gas exchange, temperature control, and removal of waste products from cells.

Assaying levels of dissolved oxygen in a microfluidic device. In some embodiments, the disclosure provides methods, systems and devices for determining the level of oxygen in a medium disposed within a microfluidic device.

In the bioproduction industry, large-scale bioreactors, including fed-batch bioreactors, are commonly used to expand and maintain cell populations that are producing a biomolecule of interest. Such large-scale bioreactors typically allow the user to monitor conditions within the bioreactor, so that they can be adjusted to maintain an optimal growth environment. Many challenges exist, however, in the selection of cell populations that will perform optimally when grown at scale, in part because selection of the cell populations happens at very small scales, when it is difficult to monitor the conditions under which the cells are growing. As a result, there is considerable expense, time and difficulty associated with identifying clonal populations having desired levels of production and growth habits when employing the currently available instrumentation and workflows. For example, developing a new antibody production line can take many months of work and cost millions of dollars in personnel, equipment and materials. The ability to screen and identify promising clones within a microfluidic device, very early in expanding populations, such as 3, 4, 5, 6, or 7 days after seeding individual founding cells, as described herein, can offer significant time and cost advantages. It has been discovered by Applicant, that the nanofluidic environment, particularly one based on sequestration pens, as described herein, provides exemplary isolation of clonal populations from each other, while permitting manipulation of the isolated populations in a manner similar to fed-batch bioreactors and the ability to obtain assay results from each individual clonal population without contamination from other clonal populations located within the microfluidic device. It has also been discovered that assays to determine the relative or absolute amount of dissolved oxygen in the medium in which the biological cells using the methods described herein, provide insights into oxygen consumption which, even when performed at an early stage of clonal expansion, can be correlated to productivity of the biological cells at more typical macroscale scale of expansion (e.g, shake flasks, etc.). Further, the ability to screen individual clones at such an early stage can also permit identification of desired clones meeting specific requirements of growth rate and/or more robust production (for example, highly productive clones which are more resistant to levels of a material in the culturing environment such as metabolic waste products or exhausted nutrients). The productivity of the biological cells can be measured in terms of grams of a biomolecule of interest produced per liter of culture, or any comparable metric suitable quantifying productivity.

Another advantage discovered by Applicant is that more complete exploration of a plurality of cells as potential founding cells for a clonal population can be made without use of excessive resources because the nanofluidic chambers (e.g., sequestration pens) described here permit simultaneous growth/assay for up to thousands of individual founding cells at the same time in extremely small volumes.

Additionally, the nanofluidic environment described here permits examination of the effects of specific conditions upon cells, with feedback from repeated assays. For example, conditions and materials, such as culture medium, more closely related to large scale production of a cellular product may be used to find and characterize the most suitable clones for further examination. In another example, diverse stimulation protocols for B-cell antibody stimulation may be examined in a more reproducible manner, and may be assayed in order to more comparably assess the benefits of one protocol over another.

Biomolecules of interest. A biomolecule of interest can comprise any molecule produced by a biological cell that a user of the methods, systems, and kits disclosed herein may wish to utilize for a particular purpose. A biomolecule of interest can include a cellular product generated and used internally or targeted to the cell membrane (in both cases “non-secreted”) or secreted by a biological micro-object, and may be a protein, a saccharide, a nucleic acid, an organic molecule having a molecular weight of less than 3 kDa, a vesicle, a virus, or any combination thereof. A biomolecule of interest may be a naturally expressed biomolecule (e.g., natively expressed) or may be a bioengineered biomolecule (e.g., a product resulting from gene insertion, deletion, modification and the like). A biomolecule of interest that is a nucleic acid may be a ribonucleic or a deoxynucleic acid, may include natural or unnatural nucleotides. A biomolecule of interest that is a virus may be a viral particle, a vector or a phage. A biomolecule of interest that is a saccharide may be a mono-, di- or polysaccharide. Non-limiting examples of saccharides may include glucose, trehalose, mannose, arabinose, fructose, ribose, xanthan or chitosan. A small, organic molecule may include but is not limited to biofuels, oils, polymers, or pharmaceutics such as macrolide antibiotics. A biomolecule of interest that is a protein can be an antibody or fragment of an antibody. A biomolecule of interest that is a protein can be a blood protein, such as an albumin, a globulin (e.g., alpha2-macroglobulin, gamma globulin, beta-2 microglobulin, haptoglobulin), a complement protein (e.g., component 3 or 4), transferrin, prothrombin, alpha 1 antitrypsin, and the like; a hormone, such as insulin, glucagon, somatostatin, growth hormone, growth factors (e.g., FGF, HGF, NGF, EGF, PDGF, TGF, Erythropoietin, IGF, TNF), follicle stimulating hormone, luteinizing hormone, leptin, and the like; a fibrous protein, such as a silk or an extracellular matrix protein (e.g., a fibronectin, laminin, collagen, elastin, vitronectin, tenascin, versican, bone sialoprotein); an enzyme, such as a metalloprotease (e.g., matrix metalloproteinase (MMP)) or other type of protease (e.g., serine protease, cysteine protease, threonine protease, aspartic protease, glutamic protease, asparagine peptide lyase), an amylase, a cellulase, a catalase, a pectinase, and the like; a bacterial, yeast, or protozoan protein; a plant protein; or a viral protein, such as a capsid or envelope protein. A biomolecule of interest that is a protein can be an antibody, fragment of an antibody, an enzyme (including but not limited to a proteolytic enzyme), an engineered (normally intracellular protein) protein, such as for example, albumin, and/or a structural protein including but not limited to silkworm silk or spider silk). This list is not limiting and any protein that may be engineered may be produced by cells that are evaluated by the methods. The biomolecule of interest may be an antibody-drug conjugate. A non-limiting example of a biomolecule of interest that may have a combination of a protein, a saccharide, a nucleic acid, an organic molecule having a molecular weight of less than 3 kDa, and/or a virus, can include a proteoglycan or glycoprotein.

The methods according to the various embodiments herein can allow for subcloning and comparative analysis of subclones, by further expanding and assaying the resultant subclone populations selected according to their oxygen consumption levels using the methods and systems described herein. This may be accomplished, for example, by moving one or more selected clonal populations to other sets of chambers (e.g., sequestration pens) within the microfluidic device and expanding each individual cell of the selected population again. In various embodiments, the method may further include a step of exporting the selected biological micro-object or the population of biological micro-objects generated therefrom to the flow region (or channel) and, optionally, out of the microfluidic device. In various embodiments, the step of export from either the chamber (e.g., sequestration pens) to the channel or from the chamber and/or channel out of the microfluidic device may be performed on each selected chamber individually (e.g., cells from a set of selected chamber may be exported in a series of export steps, one chamber at a time). Alternatively, biological micro-objects from multiple chambers can be exported simultaneously. In various embodiments, the cells which are disposed within a chamber can come from a previously assayed chamber, allowing for subcloning and comparative analysis of subclones. For example, an absolute or relative value of oxygen consumption may be used to select and expand cells. In various embodiments, all the cells from a chamber associated with a relative or absolute value representing the amount of oxygen consumption can be selected and expanded in the same chamber or other contained area of the chip. In various embodiments, one or more of the cells from the same chamber associated with a relative or absolute value representing the amount of oxygen consumption will be selected and expanded in different chambers. In various embodiments, generating a relative or absolute value of oxygen consumption may be repeatedly performed (1×, 2×, 3×, 4×, or more times) on the expanded cells.

In another embodiment, application of the disclosed methods may permit examination of the effects of specific conditions upon cells, with feedback from repeated assays. For example, conditions and materials related to large scale production of a biomolecule of interest may be used, in order to find and characterize the most suitable clones for further examination. In another example, diverse stimulation protocols for B-cell antibody stimulation may be examined in a more reproducible manner, and may be assayed in order to more comparably assess the benefits of one protocol over another.

### Dissolved Oxygen

In accordance with various embodiments, systems and methods of monitoring dissolved oxygen (DO) within a microfluidic device are disclosed. In accordance with various embodiments, the disclosed systems for monitoring dissolved oxygen during cell culture and disclosed methods for using analytical and measurement results from the assay are used for optimizing culturing and perfusion parameters for a given cell culture. Monitoring dissolved oxygen is performed by monitoring fluorescence of an oxygen sensitive dye, which is environmentally sensitive to oxygen. In various embodiments, the oxygen sensitive dye may provide a detectable signal or change in fluorescence upon binding to oxygen. However, the oxygen sensitive dye need not require a discrete binding reaction by oxygen in order to obtain a detectable signal or change in fluorescence.

The disclosed systems and methods can be applied to microfluidic-based cell cultures, e.g., a microfluidic device having chambers (e.g., sequestration pens) which open to flow regions or channels comprised by such flow regions. In various embodiments, determining the dissolved oxygen within an individual chamber (e.g., sequestration pen) in the microfluidic device, relative to that in the flow region, e.g., channel, to which the chamber is fluidically connected can be an indicator of relative amounts of oxygen consumption. Measurements of such oxygen consumption levels can be used to provide a correlation with the size of the clonal population within the chamber, e.g., more consumption correlates with more cells using oxygen. In some instances, there may be cells visible, or there may be sufficient biofouling or other biomass that may obscure how many live cells there are. In such scenario, a correlation between the amount of oxygen being consumed by the living cells can provide a quick metric for detecting the largest number of viable, growing cells. In various embodiments, the results from such correlation can help focus a cell culture experiment, for example, by guiding unpenning and export of cells from only the pens having oxygen demand above a user-defined level, e.g., according to the expected phenotype of the cells being cultured. In various embodiments, the results obtained using the aforementioned approach can be applied to maintain a certain dissolved oxygen concentration level to enhance cell expansion and viability in a given cell culture, for example, through feedback control of a rate of media perfusion, increasing the rate of media perfusion if the DO drops below a first setpoint DO level or decreasing the rate if the DO rises above a second setpoint DO level, wherein the first setpoint is lower than the second setpoint, and wherein the first and second setpoint levels may be determined according to required environmental conditions in the microfluidic device, for example, for growing cells.

In accordance with various embodiments, a method for determining oxygen consumption level in a population of biological micro-objects (e.g., cells) is provided. For example, the oxygen consumption level can be detected by diffusing soluble reporter molecules, such as a dye molecule, into the population of biological micro-objects. In accordance with various embodiments, the dye can include, but is not limited to oxygen sensitive dye “RTDP” (2 mg/L; Aldrich Cat. No. 544981-1G; CAS Registry No. 50525-27-4; Tris(2,2′-bipyridyl)-dichlororuthenium(II) hexahydrate; (Ru(BPY)₃)). In accordance with various embodiments, the dye can be used as the soluble, diffusible reporter molecule. The RTDP ruthenium complex is oxygen sensitive. The RTDP complex is a lumiphore and produces fluorescence when not quenched by local concentrations of oxygen. In various embodiments, the dye's fluorescence is diminished in the presence of dissolved oxygen, via a radiationless deactivation involving molecular interaction between oxygen and the ruthenium complex, e.g., collisional quenching, which is diffusion limited. When a sufficient concentration of oxygen is present in the local environment, e.g., in proximity to the dye, the dye's fluorescence is disrupted or quenched. Moreover, fluorescence of the dye changes based on availability of oxygen proximate to the dye. Thus, the oxygen consumption level may be measured by noting the difference in fluorescence intensity observed between a region (such as a chamber or sequestration pen described herein) in which biological micro-objects are growing (and therefore consuming oxygen) and a region (such as a flow region or channel described herein) in which biological micro-objects are not growing.

In accordance with various embodiments, the dye can include RTDP, a polycyclic aromatic hydrocarbon, a fluoranthene, a pyrene, a decacyclene, a camphorquinone, an erythrosine, a fullerene, pyrene-1-butyric acid, pyrenedecanoic acid, perfluorodecanoic acid, perylenedibutyrate, erythrosine B, fluorescent yellow, C₆₀ fullerene, C₇₀ fullerene, a ligand-metal complex, a ruthenium(II) ligand-metal complex, an iridium(III) ligand-metal complex, an osmium(II) ligand-metal complex, a rhenium(II) ligand-metal complex, a trivalent lanthanide, a metalloporphyrin, 8-hydroxy-7-iodo-5-quinolinesulfonate (“ferron”) chelated with a metal such as aluminum(III), zirconium(IV), gallium(III), or niobium(V), copper(I) complexed with pyridine or triphenylphosphine, a platinum(II) porphyrin, an intrinsically luminescent nanomaterial, a quantum dot, a carbon dot, a silicon dot, a luminescent conjugated polymer dot, a luminescent noble-metal nanoparticle, a luminescent graphene, a photon upconversion nanoparticle, a trypaflavine, a bromonaphythyl ketone, or any combination thereof.

In accordance with various embodiments, the disclosed method begins with introducing the population of biological micro-objects into a chamber of a microfluidic device having a flow region (which may include a channel) and the chamber. In accordance with various embodiments, the chamber is connected or opens to the flow region/channel. In accordance with various embodiments, the microfluidic device can include a single chamber or a plurality of chambers, and/or a single flow region/channel or a plurality of channels. In accordance with various embodiments, the chamber can be a sequestration pen or any form or type of container.

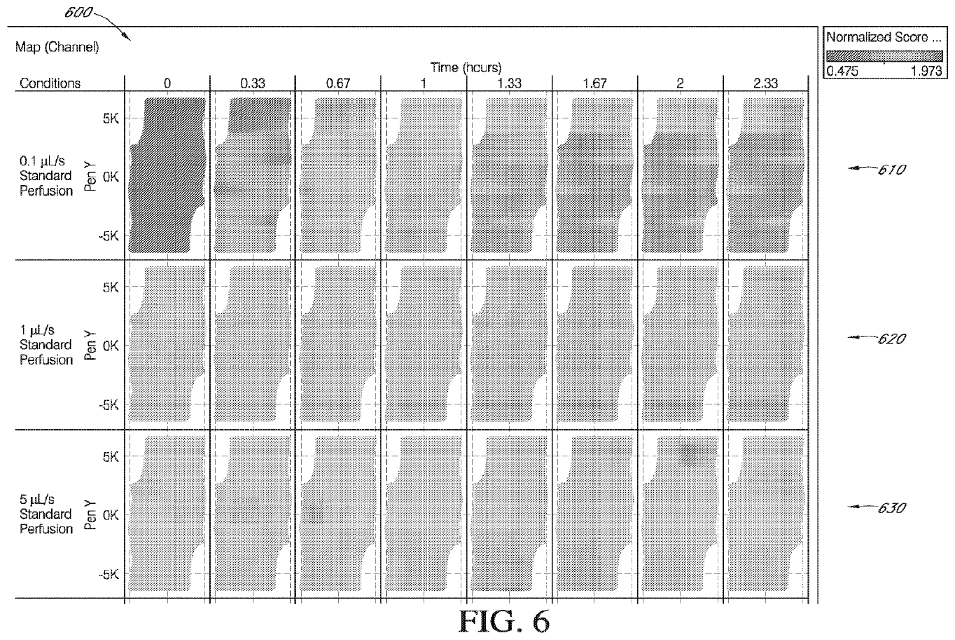

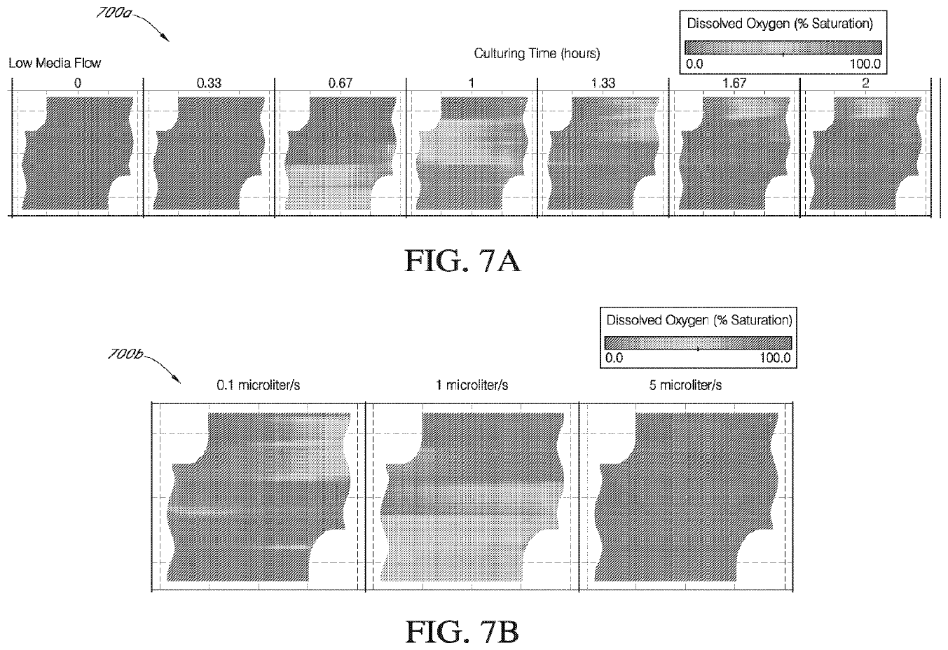

In accordance with various embodiments, the method also includes flowing a fluidic medium containing a dye, such as RTDP, and a supplied partial pressure of oxygen into the microfluidic device for a period of time. In accordance with various embodiments, fluorescence of the dye changes when the dye is in proximity to a local concentration of oxygen, for example, fluorescence level changes depending on the amount of dissolved oxygen. In accordance with various embodiments, fluorescence of the dye diminishes when the dye is quenched by a local concentration of oxygen molecules. In accordance with various embodiments, the method includes taking a fluorescence image of an area of interest (AOI) within the chamber at a time associated with a particular timestamp. The area of interest may comprise one or more portions of a fluorescence image. “Heatmaps”, as shown and described with respect to FIGS. 6, 7A, and 7B, may include data from such fluorescence images.

FIG. 6 illustrates heatmaps 600 showing normalized fluorescence intensity categorized by perfusion conditions at various times/timestamps for a microfluidic device including a plurality of chambers (e.g. sequestration pens) and channels according to some embodiments of the disclosure. The heatmaps 600 shown in FIG. 6 are of a microfluidic device that is similar to the microfluidic device shown in FIG. 1B.

In accordance with various embodiments, the culture media perfused into the microfluidic device may include any suitable culture medium, as is known in the art, for the cells under investigation, which may be any type of cells described elsewhere in this disclosure, for example animal, mammalian, human, immunological, bacterial or fungal cells. In various embodiments, perfusion may include flowing a gaseous medium. The gaseous medium may include a specified percentage of oxygen or other gases providing either optimized or test conditions for culturing the cells of interest. In some variations, the gaseous medium may include a percentage of oxygen similar to that of a standard atmosphere, e.g. about 21% oxygen (Clean Dry Air, CDA). In other variations, the gaseous medium may include a concentration of oxygen that is greater than that of CDA, such as about 25%, about 30%, about 35%, about 40%, about 45%, about 50% or more oxygen in the gaseous medium. Further, in some variations, perfusion may be performed using a mixture of liquid medium and gaseous medium. The mixture may include a mixture of liquid:gaseous media that may have a ratio of about 90:10; about 80:20; about 70:30; about 60:40; about 50:50; about 40:60; about 30:70; about 20:80, about 10:90 v/v. In some embodiments, perfusion may be performed with a mixture of liquid medium and gaseous medium which includes 80% CDA, or any percentage of oxygen as described above. In some embodiments, perfusion may be performed by performing one or more alternating perfusions of liquid medium and gaseous medium. In some embodiments, the alternating perfusions may have a duty cycle of at least about 10%, 20%, 30%, 40%, 50%, 60%, 70%, 80%, 90%, or more, wherein the percentage given represents the “on time” for perfusion of liquid medium. In some embodiments, the alternating perfusions may have a duty cycle of at most about 90%, 80%, 70%, 60%, 50%, 40%, 30%, 20%, 10%, or less, wherein the percentage given represents the “on time” for perfusion of liquid medium. In some embodiments, the alternating perfusions may have a duty cycle that ranges between any two of the preceding values. Additional details can be found below in the Experimental section, in Example 1. For example, the alternating perfusions may produce a 10% liquid to 90% gas mixture by flowing liquid medium for a period of 1 minute and then flowing gas for 9 minutes. Such a mixture would constitute a 10% duty cycle. The liquid medium may be sparged or bubbled with gas in a reagent bay to equilibrate it to a proper set point (such as 2% O₂, 21% O₂, or 40% O₂) prior to flowing the liquid medium into the microfluidic device. In various embodiments, the media source 178 comprises a sparging component in fluidic communication with a gas source providing a gas mixture with a supplied partial pressure of O₂, wherein the sparging component is operable to sparge the liquid medium with the gas mixture to provide sparged liquid medium.

As shown in FIG. 6, the different perfusion conditions shown in the heatmaps 600 include various perfusion rates, including perfusion at 0.1 microliter/s at top row 610, 1 microliter/s at middle row 620, and 5 microliter/s at bottom row 630, introducing a culture medium which causes an increase in metabolic rate in the cells under culture, e.g., causing an increase in oxygen demand by the cells. The various timestamps illustrated in the heatmaps 600 are taken at time 0 hour, 0.33 hour, 0.67 hour, 1 hour, 1.33 hour, 1.67 hour, 2 hour, and 2.33 hour. As is shown in row 610, after changing the media source, the change in metabolic rate becomes apparent after about 1.5 hour of perfusion. As observed in the top row 610, the normalized fluorescence intensity increases sharply from left to right, indicating that the perfusion rate at 0.1 microliter/s does not adequately provide sufficient oxygen content to the living cells, having more oxygen demand. The lack of oxygen becomes more severe as the experiment progresses. As observed in the middle row 620, when the flow rate was increased to 1.0 microliter/sec, the normalized fluorescence intensity does not vary as sharply from left to right and is less intense than that of the fluorescence seen in row 610 at 2.33 h. This indicates that the perfusion rate at 1 microliter/s corrects some of the deficiency and provides a more suitable amount of oxygen content to the living cells. As observed in the bottom row 630, when the flow rate is increased to 5 microliters/sec, the normalized fluorescence intensity is decreased relative to the heatmaps of rows 610 and 620, e.g., shading more towards lower fluorescence levels (“greener”). Therefore, perfusing at 5 microliter/s provides better oxygen content to the living cells when the cells are growing in a metabolic state demanding greater amounts of oxygen.

FIG. 7A illustrates the same data from FIG. 6, e.g., row 610 but the heat map presents the extent of oxygen saturation. As shown in the heatmaps 700a, perfusion at 0.1 microliter/s, using the culture medium introduced as described above, is not sufficient to provide uniform dissolved oxygen source for chambers across the entire microfluidic device, and the cells are clearly in an oxygen-deprived state by time=2 h. Additional details of the conversion of detected fluorescence to % O₂ saturation (% oxygen saturation) can be found below in the Experimental Section, in Example 1.

FIG. 7B illustrates heatmaps 700b of normalized oxygen level/consumption at various perfusion conditions at a fixed perfusion period of time of a microfluidic device including a plurality of chambers (e.g. sequestration pens) and channels according to some embodiments of the disclosure. The heatmaps 700b show dissolved oxygen levels across the microfluidic device for a period of time (e.g., 1.33 h) for different perfusion rates: 0.1 microliter/s (left heatmap), 1 microliter/s (middle heatmap), and 5 microliter/s (right heatmap), taken from the data of rows 610, 620, and 630 respectively. The trend illustrates that as the flow rate increases, the biological micro-objects are supplied with more sufficient and uniform dissolved oxygen across the microfluidic device. It is also observed that the lack of oxygen was mildly mitigated when media perfused through the side of the chip where oxygen diffused through the microfluidic circuit material, showing the horizontal stripes from the sides. These figures show that imaging the oxygen sensitive dye within the microfluidic chip can be used to monitor the sufficiency of culturing conditions, and permit adjustment of perfusion conditions and materials as needed.

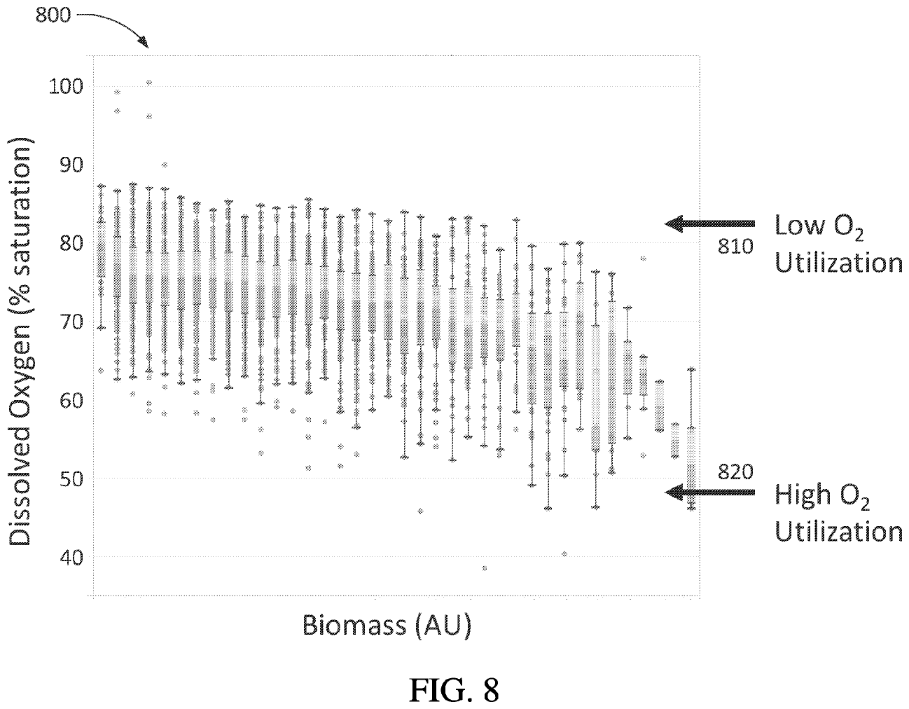

FIG. 8 is a graphical representation 800 showing normalized oxygen level/consumption as a function of biomass (e.g., a population of biological micro-objects) for each of the chambers (e.g. sequestration pens) of a microfluidic device according to some embodiments of the disclosure. The graphical representation 800 includes the dissolved oxygen level shown as a function of the biomass of the colonies in the corresponding sequestration pen, where a high dissolved oxygen level indicates a low oxygen utilization (810) by the biomass and a low dissolved oxygen level indicates a high oxygen utilization (820). In accordance with various embodiments, a clear pattern of oxygen consumption can be correlated with greater biomass, e.g., greater viable clonal population, as illustrated for example in FIG. 8. However, selection of preferred cell populations may be made using other criteria. The cells from preferred sequestration pens may be either the colonies having the highest biomass or may be from sequestration pens where the cells have the highest O₂ consumption per mass unit, e.g. where the colony may not have the most number of cells but the individual cells are consuming oxygen at the highest rate per cell. Oxygen consumption may be related to the rate of growth per cell or may be related to the rate of a cellular process such as production of a gene product or other cellular product.

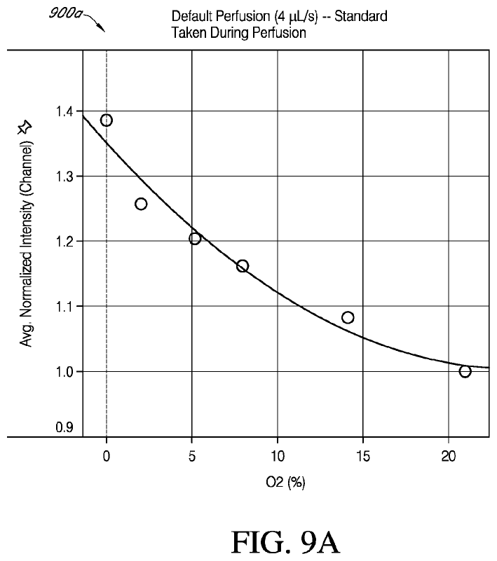

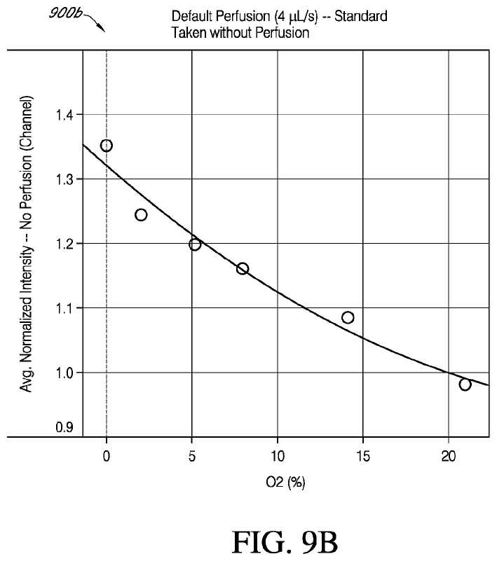

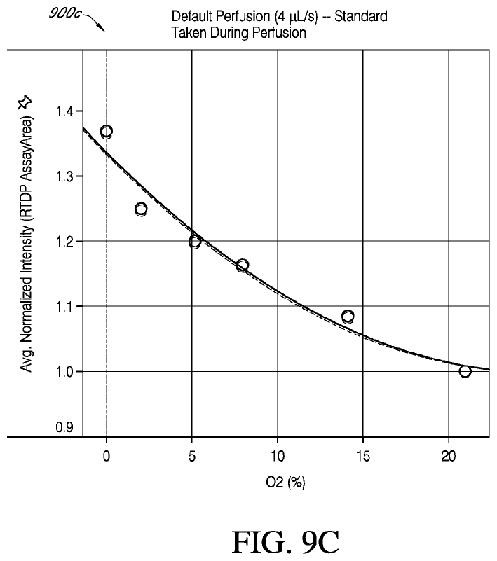

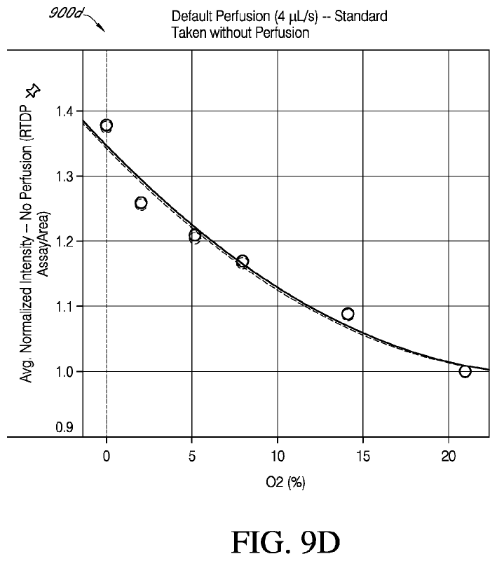

FIGS. 9A-9D illustrate the changes in observed normalized fluorescence intensity depending on perfusion conditions between the chambers and the channels. FIGS. 9A-9B illustrate normalized fluorescence intensity as a function of oxygen level in the channels of a microfluidic device according to some embodiments of the disclosure. FIG. 9A shows a plot 900a of normalized fluorescence intensity taken during standard perfusion, whereas FIG. 9B shows a plot 900b of normalized fluorescence intensity taken without perfusion.

FIGS. 9C-9D illustrate normalized fluorescence intensity as a function of oxygen level in sequestration pens of a microfluidic device according to some embodiments of the disclosure. The x axis for FIGS. 9C and 9D are oxygen level in percentage, similar to those of FIGS. 9A and 9B. FIG. 9C shows a plot 900c of normalized fluorescence intensity taken during standard perfusion, whereas FIG. 9D shows a plot 900d of normalized fluorescence intensity taken without perfusion.

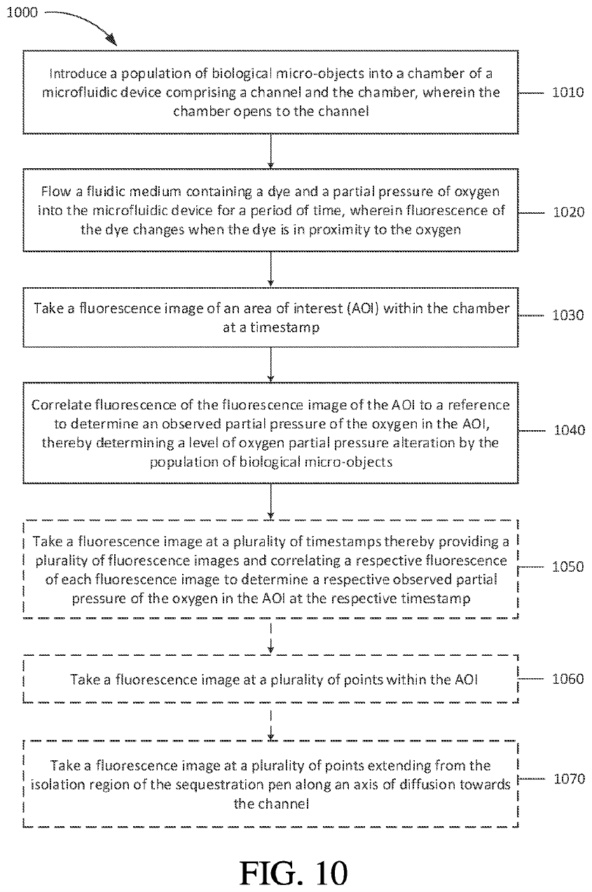

FIG. 10 illustrates a flow chart for an example method 1000 of determining oxygen consumption level in a population of biological micro-objects, according to various embodiments of the present disclosure. In accordance with various embodiments, the population of biological micro-objects are located in sequestration pens. In accordance with various embodiments, the method 1000 includes locating the population of biological micro-objects into a chamber of a microfluidic device comprising a channel and the chamber, wherein the chamber opens to the channel, at step 1010. In accordance with various embodiments, locating the population of biological micro-objects into the chamber comprises introducing the population of biological micro-objects into the chamber. In accordance with various embodiments, the method 1000 includes flowing a fluidic medium containing a dye and a supplied partial pressure of oxygen into the microfluidic device for a period of time, wherein fluorescence emitted by the dye changes when the dye encounters oxygen in the local environment, at step 1020. In accordance with various embodiments, the method 1000 includes taking a fluorescence image of an area of interest (AOI) within the chamber, at step 1030. In accordance with various embodiments, the method 1000 includes correlating fluorescence intensity of the fluorescence image of the AOI to a reference to determine an observed partial pressure of the oxygen in the AOI, thereby determining the oxygen consumption level, at step 1040. In accordance with various embodiments, the fluorescence intensity may comprise a sum of the fluorescence intensity over the AOI, a mean of the fluorescence intensity over the AOI, a median of the fluorescence intensity over the AOI, a maximum of the fluorescence intensity over the AOI, a minimum of the fluorescence intensity over the AOI, a gradient of fluorescence intensity over the AOI, or any function of fluorescence intensity over the AOI.

In accordance with various embodiments, the dye includes a soluble and diffusible dye. In accordance with various embodiments, the dye is a ruthenium complex. In accordance with various embodiments, the dye is RTDP, as described herein. In accordance with various embodiments, the dye is any dye described herein. In accordance with various embodiments, fluorescence emitted by the dye is quenched when the dye encounters oxygen in the local environment and fluoresces when the dye is not experiencing collisional quenching by oxygen in its local environment. In accordance with various embodiments, the oxygen consumption level corresponds to a decrease in the observed partial pressure of the oxygen compared to the supplied partial pressure. In accordance with various embodiments, the supplied partial pressure is measured in an area of the microfluidic device in which biological micro-objects are not growing, such as a channel described herein. In accordance with various embodiments, the fluidic medium is flowed at a flow rate of at least about 0.1 microliters per second (μL/s), 0.2 μL/s, 0.3 μL/s, 0.4 μL/s, 0.5 μL/s, 0.6 μL/s, 0.7 μL/s, 0.8 μL/s, 0.9 μL/s, 1 μL/s, 2 μL/s, 3 μL/s, 4 μL/s, 5 μL/s, 6 μL/s, 7 μL/s, 8 μL/s, 9 μL/s, 10 μL/s, or more. In accordance with various embodiments, the fluidic medium is flowed at a flow rate of at most about 10 μL/s, 9 μL/s, 8 μL/s, 7 μL/s, 6 μL/s, 5 μL/s, 4 μL/s, 3 μL/s, 2 μL/s, 1 μL/s, 0.9 μL/s, 0.8 μL/s, 0.7 μL/s, 0.6 μL/s, 0.5 μL/s, 0.4 μL/s, 0.3 μL/s, 0.2 μL/s, 0.1 μL/s, or less.

In accordance with various embodiments, the fluidic medium is flowed at a flow rate ranging between any two of the preceding values. In accordance with various embodiments, the fluidic medium is flowed at a flow rate ranging between 0.1 microliter/s and 10 microliters/s. In accordance with various embodiments, the population of biological micro-objects in the chamber of the microfluidic device is cultured under aerobic conditions comprising flowing a fluidic medium comprising a supplied partial pressure of oxygen. In accordance with some embodiments, the supplied partial pressure of oxygen is at least about 0.01 bar, 0.02 bar, 0.3 bar, 0.04 bar, 0.05 bar, 0.06 bar, 0.07 bar, 0.08 bar, 0.09 bar, 0.1 bar, 0.15 bar, 0.2 bar, 0.25 bar, 0.3 bar, 0.4 bar, 0.5 bar, 0.6 bar, 0.7 bar, 0.8 bar, 0.9 bar, 1 bar, or more. In accordance with various embodiments, the supplied partial pressure of oxygen is at most about 1 bar, 0.9 bar, 0.8 bar, 0.7 bar, 0.6 bar, 0.5 bar, 0.4 bar, 0.3 bar, 0.25 bar, 0.2 bar, 0.15 bar, 0.1 bar, 0.09 bar, 0.08 bar, 0.07 bar, 0.06 bar, 0.05 bar, 0.04 bar, 0.03 bar, 0.02 bar, 0.01 bar, or less. In accordance with various embodiments, the supplied partial pressure of oxygen ranges between any two of the preceding values.

In accordance with various embodiments, the AOI is disposed within the chamber at a location wherein transference of components of the fluidic medium flowing in the channel is dominated by diffusion. In accordance with various embodiments, “dominated by diffusion” means that diffusion is the primary mechanism for transference of components of the fluidic medium flowing in the channel, as compared to all other non-diffusive transport mechanisms. In accordance with various embodiments, “dominated by diffusion” means that diffusion contributes at least about 75%, 80%, 85%, 90%, 95%, 99%, or more of the transference of components of the fluidic medium flowing in the channel. In accordance with various embodiments, “dominated by diffusion” means that diffusion contributes at most about 99%, 95%, 90%, 85%, 80%, 75%, or less of the transference of components of the fluidic medium flowing in the channel. In accordance with various embodiments, “dominated by diffusion” means that diffusion contributes a range of the transference of components of the fluidic medium flowing in the channel that is defined by any two of the preceding values. In accordance with various embodiments, “dominated by diffusion” means that transference of components of the fluidic medium flowing in the channel occurs substantially only by diffusion.

In accordance with various embodiments, the AOI may contain no biological micro-objects. In accordance with various embodiments, the fluidic medium includes a liquid medium, a gaseous medium or a mixture thereof. In accordance with various embodiments, the fluidic medium includes a mixture of a liquid medium and a gaseous medium. In accordance with various embodiments, the mixture of the liquid medium and the gaseous medium includes at least a 10:90, 15:85, 20:80, 25:75, 30:70, 35:65, or 40:60 v/v ratio of the liquid medium to the gaseous medium. In accordance with various embodiments, the mixture of the liquid medium and the gaseous medium includes at most a 40:60, 35:65, 30:70, 25:75, 20:80, 15:85, or 10:90 v/v ratio of the liquid medium to the gaseous medium. In accordance with various embodiments, the mixture of the liquid medium and the gaseous medium includes a v/v ratio that ranges between any two of the preceding values. In accordance with various embodiments, the medium includes a liquid medium saturated with a selected supplied partial pressure of the oxygen. In some embodiments, alternating perfusions of liquid medium and gaseous medium are supplied. In some embodiments, the alternating perfusions may have a duty cycle of at least about 10%, 20%, 30%, 40%, 50%, 60%, 70%, 80%, 90%, or more. In some embodiments, the alternating perfusions may have a duty cycle of at most about 90%, 80%, 70%, 60%, 50%, 40%, 30%, 20%, 10%, or less. In some embodiments, the alternating perfusions may have a duty cycle that ranges between any two of the preceding values. Additional details can be found below in the Experimental section, in Example 1.

In accordance with various embodiments, correlating fluorescence of the fluorescence image of the AOI to determine an observed partial pressure of the oxygen at the AOI further comprises identifying an intensity of the fluorescence on a reference curve correlating fluorescence intensities of the dye with supplied partial pressures of the oxygen. In accordance with various embodiments, the method 1000 further includes constructing a reference curve correlating an observed intensity of fluorescence of the dye with a supplied partial pressure of oxygen. In accordance with various embodiments, constructing the reference curve further includes flowing a liquid medium through the channel of the microfluidic device for a first reference period of time, wherein the liquid medium comprises the dye and a first supplied partial pressure of the oxygen, detecting a first fluorescence intensity of the dye within a selected region of the microfluidic device, flowing a second liquid medium through the channel of the microfluidic device for a second reference period of time, wherein the second liquid medium comprises the dye and a second supplied partial pressure of the oxygen, detecting a second fluorescence intensity of the dye within the selected region of the microfluidic device, and correlating each of the first and the second fluorescence intensities with the supplied partial pressure of the oxygen.

In accordance with various embodiments, constructing the reference curve further includes flowing liquid media comprising the dye and successive supplied partial pressures of the oxygen at successive points in time; detecting successive fluorescence intensities of the dye at the successive points in time; and correlating each of the fluorescence intensities with a supplied partial pressure of the oxygen.

In accordance with various embodiments, the microfluidic device does not contain any biological micro-objects while constructing the reference curve. In accordance with various embodiments, the selected supplied partial pressure of oxygen is at least about 0.01 bar, 0.02 bar, 0.03 bar, 0.04 bar, 0.05 bar, 0.06 bar, 0.07 bar, 0.08 bar, 0.09 bar, 0.1 bar, 0.11 bar, 0.12 bar, 0.13 bar, 0.14 bar, 0.15 bar, 0.16 bar, 0.17 bar, 0.18 bar, 0.19 bar, 0.2 bar, 0.21 bar, 0.22 bar, 0.23 bar, 0.24 bar, 0.25 bar, or more. In accordance with various embodiments, the selected supplied partial pressure of oxygen is at most about 0.25 bar, 0.24 bar, 0.23 bar, 0.22 bar, 0.21 bar, 0.2 bar, 0.19 bar, 0.18 bar, 0.17 bar, 0.16 bar, 0.15 bar, 0.14 bar, 0.13 bar, 0.12 bar, 0.11 bar, 0.1 bar, 0.09 bar, 0.08 bar, 0.07 bar, 0.06 bar, 0.05 bar, 0.04 bar, 0.03 bar, 0.02 bar, 0.01 bar, or less. In accordance with various embodiments, the selected supplied partial pressure of oxygen ranges between any two of the preceding values. In accordance with various embodiments, the selected supplied partial pressure of oxygen ranges from about 0.02 bar to about 0.21 bar. In accordance with various embodiments, the method 1000 includes detecting fluorescence intensities associated with at least about three, four, five, or more different supplied partial pressures of the oxygen. In accordance with various embodiments, the method 1000 includes detecting fluorescence intensities associated with at most about five, four, three, or fewer supplied different partial pressures of the oxygen. In accordance with various embodiments, the method 1000 includes detecting a number of fluorescence intensities associated with a number of different supplied partial pressures of the oxygen that ranges between any two of the preceding values. In accordance with various embodiments, the fluorescence image is taken under a perfusion condition. In accordance with various embodiments, the perfusion condition comprises a constant flow, a steady state flow, a pre-programmed flow with one or more defined flow rates, or zero flow after equilibrium.

In accordance with various embodiments, the microfluidic device includes a plurality of chambers, and the method 1000 further includes introducing the population of biological micro-objects into the plurality of chambers. In accordance with various embodiments, the microfluidic device includes a plurality of channels, and the method 1000 further includes introducing the population of biological micro-objects into the plurality of channels.

In accordance with various embodiments, flowing the fluidic medium and taking the fluorescence image are performed at a selected temperature. In accordance with various embodiments, the temperature is from about 20° C. to about 40° C. In accordance with various embodiments, the temperature is from about 28° C. to about 30° C. However, the method is not so limited. The cells may alternatively be cultured at other temperatures, such as at least about 10° C., 15° C., 20° C., 25° C., 30° C., 35° C., 40° C., 45° C., 50° C., 55° C., 60° C., 65° C., 70° C., or higher. Further, the cells may be cultured at temperature of at most about 70° C., 65° C., 60° C., 55° C., 50° C., 45° C., 40° C., 35° C., 30° C., 25° C., 20° C., 15° C., 10° C., or less. In accordance with various embodiments, the cells may be cultured at a temperature that ranges between any two of the preceding values. In accordance with various embodiments, flowing the fluidic medium and taking the fluorescence image is performed at a selected pH. In accordance with various embodiments, the pH may be at least about 3.0, 4.0, 5.0, 6.0, 7.0, 8.0, 9,0, or more. In accordance with various embodiments, the pH may be at most about 9.0, 8.0, 7.0, 6,0, 5.0, 4.0, 3.0, or less. In accordance with various embodiments, the pH may range between any two of the preceding values. In accordance with various embodiments, the pH is from about 3.0 to about 9.0.

In accordance with various embodiments, the method 1000 optionally includes taking a plurality of fluorescence images at a plurality of times/timestamps correlating a respective fluorescence of each fluorescence image to determine a respective observed partial pressure of the oxygen in the AOI at the respective timestamp, at step 1050. In accordance with various embodiments, the method 1000 optionally includes taking a plurality of fluorescence images at a plurality of points within the AOI, at step 1060.

In accordance with various embodiments, the chamber includes a sequestration pen, wherein the sequestration includes an isolation region and a connection region, and wherein the connection region has a proximal opening to the channel and a distal opening to the isolation region. In accordance with various embodiments, the isolation region includes a single opening to the connection region. In accordance with various embodiments, the population of biological micro-objects is disposed within the isolation region of the sequestration pen.

In accordance with various embodiments, the method 1000 optionally includes taking a plurality of fluorescence images at a plurality of points extending from the isolation region of the sequestration pen along an axis of diffusion towards the channel, at step 1070. In accordance with various embodiments, the AOI includes at least part of the connection region. In various embodiments the area of interest comprises at least a portion of the sequestration pen aligned along an axis of diffusion from within the sequestration pen out into the flow region. In various embodiments the area of interest can be partitioned into a plurality of segments and, in some embodiments, an average signal can be computed for each of the segments.

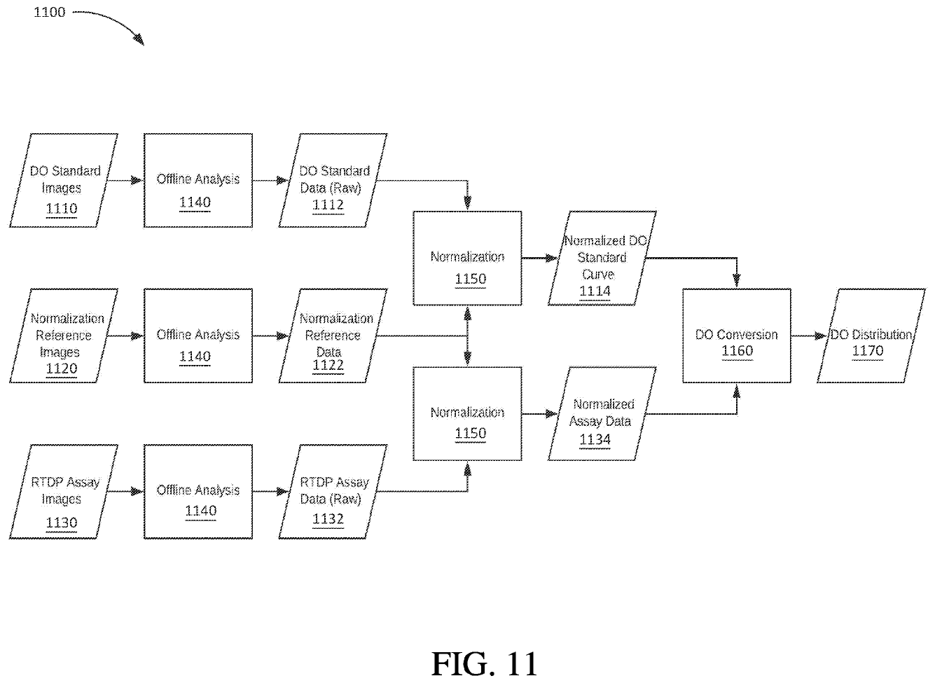

FIG. 11 illustrates an example approach 1100 of converting acquired fluorescence images into data for correlating fluorescence of an AOI to a reference to determine the dissolved oxygen level accordance with some embodiments of the present disclosure. In various embodiments, the approach 1100 includes performing dissolved oxygen (DO) Standard Testing with multiple oxygen setpoints as described with respect to FIGS. 6-10. The fluorescence images taken in the Standard Testing is labeled as DO Standard Images 1110 as shown in FIG. 11. In various implementations, the DO Standard Images of air saturated RTDP media are selected to be used for normalization for imaging correction at a later time. Such images are named Normalization Reference Images 1120.

In various embodiments, the approach 1100 includes performing the DO assay with RTDP during cell culture as described with respect to FIGS. 6-10. Such data are referred to as RTDP Assay Images 1130, as shown in FIG. 11.

In various embodiments, the approach 1100 includes using an Offline Analysis operation 1140 to measure the mean intensities in the AOIs for all fluorescence images. As illustrated in FIG. 11, using the Offline Analysis operation 1140, the DO Standard Images 1110, Normalization Reference Images 1120, and RTDP Assay Images 1130 are converted into DO Standard Data (Raw) 1112, Normalization Reference Data 1122, and RTDP Assay Data (Raw) 1132, respectively. In accordance with various embodiments, the customized AOIs and configurable parameters used in the Offline Analysis operation 1140 are identical so that the processed data from the three individual sequences are comparable and can be further processed and analyzed.

As illustrated in FIG. 11, the approach 1100 further includes normalizing the DO Standard Data (Raw) 1112 and RTDP Assay Data (Raw) 1132 by the Normalization Reference Data 1122 via Normalization 1150. Once the DO Standard Data (Raw) 1112 and RTDP Assay Data (Raw) 1132 are normalized, the approach 1100 further includes dividing the mean intensity of DO Standard Data (Raw) 1112 and RTDP Assay Data (Raw) 1132 in the AOIs in each chamber (e.g., sequestration pen) by the corresponding mean intensity in the Normalization Reference Data 1122 to generate a Normalized DO Standard Curve 1114 and Normalized Assay Data 1134. The normalization process can suppress most of the imaging artifacts, such as for example, nonuniform illumination, flat-field effect, etc.

After normalizing, the approach 1100 further includes conversion of the Normalized Assay Data 1134 into percentage dissolved oxygen (% DO) using the generated Normalized DO Standard Curve 1114 via DO Conversion 1160. Once the DO Conversion 1160 is obtained, the DO Distribution 1170 is calculated.

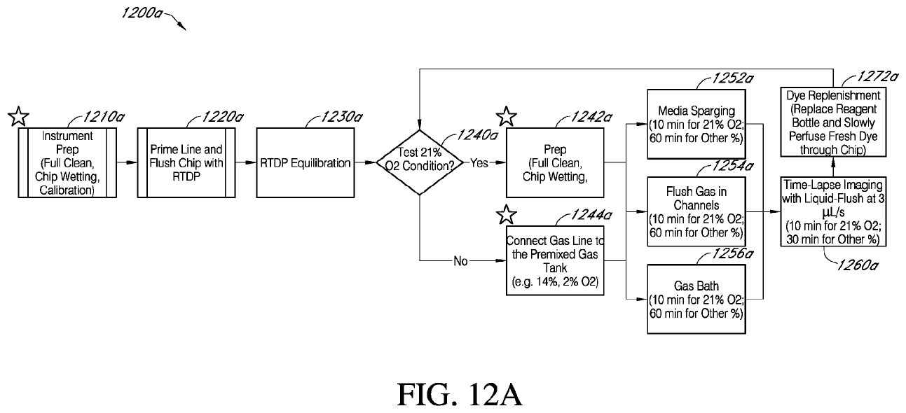

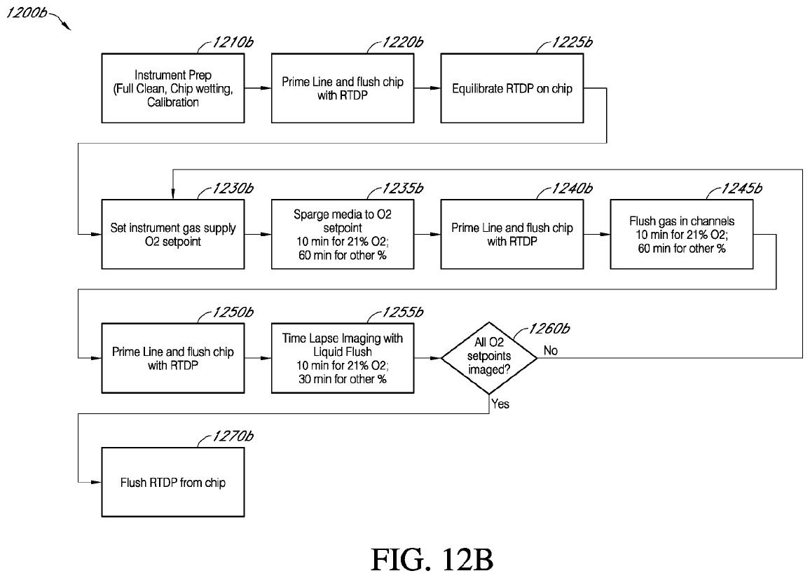

FIG. 12A illustrates a first example approach 1200a of generating a DO standard curve. In accordance with various embodiments, the approach 1200a includes an instrument preparation operation 1210a. In accordance with various embodiments, the instrument preparation operation includes a full clean of the flow system, microfluidic device or chip wetting.

In accordance with various embodiments, after the instrument preparation operation, the approach 1200a includes a priming operation 1220a. In accordance with various embodiments, the priming operation includes a flow system line priming operation and flushing of the microfluidic device or chip with RTDP.

In accordance with various embodiments, after the priming operation, the approach 1200a includes a RTDP equilibration operation 1230a. In accordance with various embodiments, the RTDP equilibration operation includes allowing the RTDP to equilibrate within the microfluidic device or chip.

In accordance with various embodiments, after the RTDP equilibration operation, the approach 1200a includes an oxygen setpoint determination operation 1240a. In accordance with various embodiments, the oxygen setpoint determination operation includes determining whether the current oxygen setpoint is approximately 21%. In accordance with various embodiments, if the current oxygen setpoint is approximately 21%, the flow system is connected to a 21% O₂ source (such as a 21% O₂ gas cylinder) at operation 1242a. In accordance with various embodiments, if the current oxygen setpoint is not approximately 21%, the flow system is connected to an O₂ source having an O₂ concentration different from 21% at operation 1244a.

In accordance with various embodiments, after the oxygen setpoint determination operation, the approach 1200a includes a media sparging operation 1252a, a gas flush operation 1254a, and/or a gas bath operation 1256a. In accordance with various embodiments, the media sparging operation includes sending a gas mixture containing oxygen from the O₂ source to a liquid medium and allowing the gas mixture to bubble into the liquid medium for a first period of time until the liquid medium attains a desired equilibrium oxygen setpoint. In accordance with various embodiments, the first period of time is at least about 1 minute (min), 2 min, 3 min, 4 min, 5 min, 6 min, 7 min, 8 min, 9 min, 10 min, 15 min, 20 min, 25 min, 30 min, 35 min, 40 min, 45 min, 50 min, 55 min, 60 min, or more. In accordance with various embodiments, the first period of time is at most about 60 min, 55 min, 50 min, 45 min, 40 min, 35 min, 30 min, 25 min, 20 min, 15 min, 10 min, 9 min, 8 min, 7 min, 6 min, 5 min, 4 min, 3 min, 2 min, 1 min, or less. In accordance with various embodiments, the first period of time ranges between any two of the preceding values. In accordance with various embodiments, the gas flush operation includes flushing O₂ through channels of the microfluidic device or chip for a second period of time. In accordance with various embodiments, the second period of time is at least about 1 minute (min), 2 min, 3 min, 4 min, 5 min, 6 min, 7 min, 8 min, 9 min, 10 min, 15 min, 20 min, 25 min, 30 min, 35 min, 40 min, 45 min, 50 min, 55 min, 60 min, or more. In accordance with various embodiments, the second period of time is at most about 60 min, 55 min, 50 min, 45 min, 40 min, 35 min, 30 min, 25 min, 20 min, 15 min, 10 min, 9 min, 8 min, 7 min, 6 min, 5 min, 4 min, 3 min, 2 min, 1 min, or less. In accordance with various embodiments, the second period of time ranges between any two of the preceding values. In accordance with various embodiments, the gas bath operation comprises surrounding the microfluidic device or chip in an O₂ gas bath, as described herein, for a third period of time. In accordance with various embodiments, the third period of time is at least about 1 minute (min), 2 min, 3 min, 4 min, 5 min, 6 min, 7 min, 8 min, 9 min, 10 min, 15 min, 20 min, 25 min, 30 min, 35 min, 40 min, 45 min, 50 min, 55 min, 60 min, or more. In accordance with various embodiments, the third period of time is at most about 60 min, 55 min, 50 min, 45 min, 40 min, 35 min, 30 min, 25 min, 20 min, 15 min, 10 min, 9 min, 8 min, 7 min, 6 min, 5 min, 4 min, 3 min, 2 min, 1 min, or less. In accordance with various embodiments, the third period of time ranges between any two of the preceding values.

In accordance with various embodiments, after the media sparging operation, gas flush operation, and gas bath operation, the approach 1200a includes a time-lapse imaging operation 1260a. In accordance with various embodiments, the time-lapse imaging operation comprises acquiring a plurality of fluorescence images of the microfluidic device or chip in the presence of a liquid flush. In accordance with various embodiments, the liquid flush includes flowing liquid through the microfluidic device or chip at a flow rate for a fourth period of time. In accordance with various embodiments, the flow rate is at least about 1 microliter per second (μL/s), 2 μL/s, 3 μL/s, 4 μL/s, μL/s, 5 μL/s, 6 μL/s, 7 μL/s, 8 μL/s, 9 μL/s, 10 μL/s, or more. In accordance with various embodiments, the flow rate is at most about 10 μL/s, 9 μL/s, 8 μL/s, 7 μL/s, 6 μL/s, 5 μL/s, 4 μL/s, 3 μL/s, 2 μL/s, 1 μL/s, or less. In accordance with various embodiments, the flow rate ranges between any two of the preceding values. In accordance with various embodiments, the fourth period of time is at least about 1 minute (min), 2 min, 3 min, 4 min, 5 min, 6 min, 7 min, 8 min, 9 min, 10 min, 15 min, 20 min, 25 min, 30 min, 35 min, 40 min, 45 min, 50 min, 55 min, 60 min, or more. In accordance with various embodiments, the fourth period of time is at most about 60 min, 55 min, 50 min, 45 min, 40 min, 35 min, 30 min, 25 min, 20 min, 15 min, 10 min, 9 min, 8 min, 7 min, 6 min, 5 min, 4 min, 3 min, 2 min, 1 min, or less. In accordance with various embodiments, the fourth period of time ranges between any two of the preceding values.

In accordance with various embodiments, after the time-lapse imaging operation, the process 1200a includes a dye replenishment operation 1272a. In accordance with various embodiments, the dye replenishment operation comprises perfusing fresh dye (such as RTDP) through the microfluidic device or chip.

In accordance with various embodiments, after the dye replenishment operation, the process 1200a includes repeating any of operations 1240a, 1242a, 1244a, 1252a, 1254a, 1260a, 1262a, and 1272a. In accordance with various embodiments, the process includes repeating any of operations 1240a, 1242a, 1244a, 1252a, 1254a, 1260a, 1262a, and 1272a at least about 1, 2, 3, 4, 5, 6, 7, 8, 9, 10, or more times. In accordance with various embodiments, the process includes repeating any of operations 1240a, 1242a, 1244a, 1252a, 1254a, 1260a, 1262a, and 1272a at most about 10, 9, 8, 7, 6, 5, 4, 3, 2, or 1 times. In accordance with various embodiments, the process includes repeating any of operations 1240a, 1242a, 1244a, 1252a, 1254a, 1260a, 1262a, and 1272a a number of times that ranges between any two of the preceding values. In this manner, the RTDP fluorescence signal may be imaged for a variety of desired oxygen setpoints. The fluorescence signals associated with each oxygen setpoint may be collected and used to fit a DO standard curve. The RTDP fluorescence signals in areas surrounding biological micro-objects may then be compared to this DO standard curve to determine the associated DO level in the areas surrounding the biological micro-objects, as described herein.

FIG. 12B illustrates a second example approach 1200b of generating a DO standard curve. In accordance with various embodiments, the approach 1200b includes an instrument preparation operation 1210b. In accordance with various embodiments, the instrument preparation operation 1210b is similar to instrument preparation operation 1210a described herein with respect to FIG. 12A. In accordance with various embodiments, the instrument preparation operation includes a full clean of the flow system, microfluidic device or chip wetting, and calibration.

In accordance with various embodiments, after the instrument preparation operation, the approach 1200b includes a priming operation 1220b. In accordance with various embodiments, the priming operation 1220b is similar to priming operation 1220a described herein with respect to FIG. 12A. In accordance with various embodiments, the priming operation includes a flow system line priming operation and flushing of the microfluidic device or chip with RTDP.

In accordance with various embodiments, after the priming operation, the approach 1200b includes a RTDP equilibration operation 1225b. In accordance with various embodiments, the RTDP equilibration operation 1225b is similar to RTDP equilibration operation 1230a described herein with respect to FIG. 12A. In accordance with various embodiments, the RTDP equilibration operation includes allowing the RTDP to equilibrate within the microfluidic device or chip. In accordance with various embodiments, the RTDP equilibration operation includes slowly perfusing RTDP through the microfluidic device or chip to allow RTDP to diffuse into sequestration pens until equilibrium RTDP concentration is reached.

In accordance with various embodiments, after the RTDP equilibration operation, the approach 1200b includes an oxygen setpoint operation 1230b. In accordance with various embodiments, the oxygen setpoint operation 1230b includes receiving an oxygen setpoint supplied by a user. In accordance with various embodiments, the oxygen setpoint operation includes sending a signal to a multi-gas controller to mix gases from supply tanks until the oxygen content of the gas mixture reaches the desired oxygen setpoint. In accordance with various embodiments, the oxygen setpoint operation includes sending the gas mixture to the microfluidic device or chip, or elsewhere in the system, for example, for liquid media sparging.

In accordance with various embodiments, after the oxygen setpoint operation 1230b, the approach 1200b includes a media sparging operation 1235b. In accordance with various embodiments, the media sparging operation 1235b is similar to media sparging operations 1252a described herein with respect to FIG. 12A. In accordance with various embodiments, the media sparging operation includes sending the gas mixture to a liquid medium (which includes RTDP) and allowing the gas mixture to bubble into the liquid medium for a first period of time until the liquid medium attains a desired equilibrium oxygen setpoint. In accordance with various embodiments, the first period of time is at least about 1 minute (min), 2 min, 3 min, 4 min, 5 min, 6 min, 7 min, 8 min, 9 min, 10 min, 15 min, 20 min, 25 min, 30 min, 35 min, 40 min, 45 min, 50 min, 55 min, 60 min, or more. In accordance with various embodiments, the first period of time is at most about 60 min, 55 min, 50 min, 45 min, 40 min, 35 min, 30 min, 25 min, 20 min, 15 min, 10 min, 9 min, 8 min, 7 min, 6 min, 5 min, 4 min, 3 min, 2 min, 1 min, or less. In accordance with various embodiments, the first period of time ranges between any two of the preceding values.

In accordance with various embodiments, after the media sparging operation 1235b, the approach 1200b includes a first chip flush operation 1240b. In accordance with various embodiments, the chip flush operation comprises flushing the liquid medium (which has been oxygenated by the gas sparging operation) through the microfluidic device or chip.

In accordance with various embodiments, after the chip flush operation 1240b, the approach 1200b includes a gas flush operation 1245b. In accordance with various embodiments, the gas flush operation 1245b is similar to gas flush operation 1254a described herein with respect to FIG. 12A. In accordance with various embodiments, the gas flush operation includes flushing the gas mixture through channels of the microfluidic device or chip for a second period of time. In accordance with various embodiments, the second period of time is at least about 1 minute (min), 2 min, 3 min, 4 min, 5 min, 6 min, 7 min, 8 min, 9 min, 10 min, 15 min, 20 min, 25 min, 30 min, 35 min, 40 min, 45 min, 50 min, 55 min, 60 min, or more. In accordance with various embodiments, the second period of time is at most about 60 min, 55 min, 50 min, 45 min, 40 min, 35 min, 30 min, 25 min, 20 min, 15 min, 10 min, 9 min, 8 min, 7 min, 6 min, 5 min, 4 min, 3 min, 2 min, 1 min, or less. In accordance with various embodiments, the second period of time ranges between any two of the preceding values.

In accordance with various embodiments, after the gas flush operation 1245b, the approach 1200b includes a second chip flush operation 1250b. In accordance with various embodiments, the second chip flush operation 1250b is similar to first chip flush operation 1240b described herein with respect to FIG. 12B.

In accordance with various embodiments, after the second chip flush operation 1250b, the approach 1200b comprises a time-lapse imaging operation 1255b. In accordance with various embodiments, the time-lapse imaging operation 1255b is similar to time-lapse imaging operation 1260a described herein with respect to FIG. 12A. In accordance with various embodiments, the time-lapse imaging operation comprises acquiring a plurality of fluorescence images of the microfluidic device or chip in the presence of a liquid flush. In accordance with various embodiments, the liquid flush includes flowing liquid through the microfluidic device or chip at a flow rate for a third period of time. In accordance with various embodiments, the flow rate is at least about 1 microliter per second (μμL/s), 2 μL/s, 3 μL/s, 4 μL/s, μL/s, 5 μL/s, 6 μL/s, 7 μL/s, 8 μL/s, 9 μL/s, 10 μL/s, or more. In accordance with various embodiments, the flow rate is at most about 10 μL/s, 9 μL/s, 8 μL/s, 7 μL/s, 6 μL/s, 5 μL/s, 4 μL/s, 3 μL/s, 2 μL/s, 1 μL/s, or less. In accordance with various embodiments, the flow rate ranges between any two of the preceding values. In accordance with various embodiments, the third period of time is at least about 1 minute (min), 2 min, 3 min, 4 min, 5 min, 6 min, 7 min, 8 min, 9 min, 10 min, 15 min, 20 min, 25 min, 30 min, 35 min, 40 min, 45 min, 50 min, 55 min, 60 min, or more. In accordance with various embodiments, the third period of time is at most about 60 min, 55 min, 50 min, 45 min, 40 min, 35 min, 30 min, 25 min, 20 min, 15 min, 10 min, 9 min, 8 min, 7 min, 6 min, 5 min, 4 min, 3 min, 2 min, 1 min, or less. In accordance with various embodiments, the third period of time ranges between any two of the preceding values.

In accordance with various embodiments, after the time-lapse imaging operation 1255b, the approach 1200b includes an oxygen setpoint feedback operation 1260b. In accordance with various embodiments, the oxygen setpoint feedback operation includes determining whether all desired oxygen setpoints have been imaged. If no, the operations 1230b, 1235b, 1240b, 1245b, 1250b, and 1255b are repeated one or more times for one or more desired oxygen setpoints. In this manner, the RTDP fluorescence signal may be imaged for a variety of desired oxygen setpoints. If yes, the microfluidic device or chip may be flushed with RTDP at operation 1270b. The fluorescence signals associated with each oxygen setpoint may be collected and used to fit a DO standard curve.

The RTDP fluorescence signals in areas of interest proximate to biological micro-objects may then be compared to this DO standard curve to determine the associated DO level in the areas surrounding the biological micro-objects, as described herein.

The approaches 1200a and 1200b described herein with respect to FIGS. 12A and 12B represent two possible approaches to generating a DO standard curve. One skilled in the art will recognize that other methods may also be effective for generating the DO standard curve.

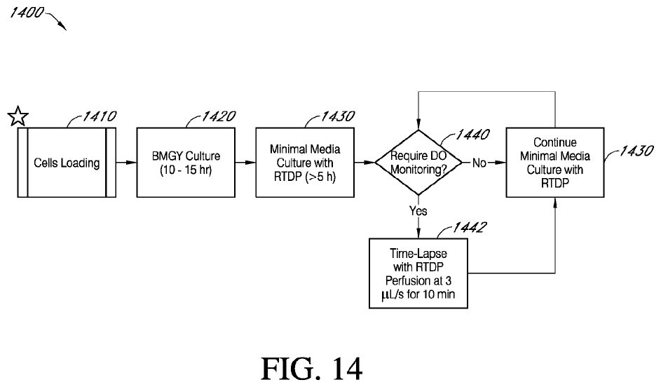

FIG. 14 illustrates a first example approach 1400 of performing a DO perfusion assay. In accordance with various embodiments, the approach 1400 includes a cell loading operation 1410. In accordance with various embodiments, the cell loading operation comprises loading cells into sequestration pens of the microfluidic device or chip.

In accordance with various embodiments, after the cell loading operation, the approach 1400 includes a first culture operation 1420. In accordance with various embodiments, the first culture operation comprises culturing the cells in the presence of BMGY growth medium, or other liquid growth medium sufficient to support growth of cells. In accordance with various embodiments, BMGY growth medium comprises peptone, yeast extract, biotin, yeast nitrogen base, potassium phosphate monobasic, potassium phosphate dibasic, and glycerol. In accordance with various embodiments, the first culture operation comprises culturing the cells in the presence of BMMY growth medium. In accordance with various embodiments, BMMY growth medium comprises peptone, yeast extract, biotin, yeast nitrogen base, potassium phosphate monobasic, potassium phosphate dibasic, and methanol. In accordance with various embodiments, the first culture operation comprises culturing the cells in the presence of Bird growth medium. In accordance with various embodiments, Bird growth medium comprises ammonium sulfate, monopotassium phosphate, magnesium sulfate heptahydrate, succinic acid, biotin, calcium pantothenate, nicotinic acid, myoinositol, thiamine hydrochloride, pyridoxol hydrochloride, p-aminobenzoic acid, ethylenediaminetetraacetic acid (EDTA), zinc sulfate heptahydrate, copper sulfate anhydrous, manganese chloride tetrahydrate, cobalt chloride hexahydrate, sodium molybdenite dihydrate, iron sulfate heptahydrate, iron chloride hexahydrate, calcium chloride dihydrate, and lysine. In accordance with various embodiments, the first culture operation comprises culturing the cells in the presence of Delft growth medium. In accordance with various embodiments, Delft growth medium comprises ammonium sulfate, monopotassium phosphate, magnesium sulfate heptahydrate, glucose, EDTA, zinc sulfate heptahydrate, manganese chloride dihydrate, cobalt chloride hexahydrate, copper sulfate pentahydrate, sodium molybdenite dihydrate, calcium chloride dihydrate, iron sulfate heptahydrate, boric acid, potassium iodide, biotin, p-aminobenzoic acid, nicotinic acid, calcium pantothenate, pyridoxine hydrochloride, thiamine hydrochloride, and myoinositol. In accordance with various embodiments, the first culture operation comprises culturing the cells in LSM growth medium. In accordance with various embodiments, LSM growth medium comprises potassium phosphate monobasic, ammonium sulfate, calcium sulfate dihydrate, magnesium sulfate heptahydrate, sodium citrate, glycerol or methanol, vitamin mix, and PTM4 solution. In accordance with various embodiments, the first culture operation comprises culturing the cells in FM22 growth medium. In accordance with various embodiments, FM22 growth medium comprises potassium phosphate monobasic, ammonium sulfate, calcium sulfate dihydrate, magnesium sulfate heptahydrate, PTM4 solution, and dextrose or glycerol.

In accordance with various embodiments, the first culture operation comprises culturing the cells for at least about 1 hour (h), 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, or more. In accordance with various embodiments, the first culture operation comprises culturing the cells for at most about 24 h, 23 h, 22 h, 21 h, 20 h, 19 h, 18 h, 17 h, 16 h, 15 h, 14 h, 13 h, 12 h, 11 h, 10 h, 9 h, 8 h, 7 h, 6 h, 5 h, 4 h, 3 h, 2 h, 1 h, or less. In accordance with various embodiments, the first culture operation comprises culturing the cells for a period of time that ranges between any two of the preceding values.

In accordance with various embodiments, after the first culture operation, the approach 1400 includes a second culture operation 1430. In accordance with various embodiments, the second culture operation comprises culturing the cells in minimal growth medium with RTDP. In accordance with various embodiments, the second culture operation comprises culturing the cells for at least about 1 hour (h), 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, or more. In accordance with various embodiments, the second culture operation comprises culturing the cells for at most about 24 h, 23 h, 22 h, 21 h, 20 h, 19 h, 18 h, 17 h, 16 h, 15 h, 14 h, 13 h, 12 h, 11 h, 10 h, 9 h, 8 h, 7 h, 6 h, 5 h, 4 h, 3 h, 2 h, 1 h, or less. In accordance with various embodiments, the second culture operation comprises culturing the cells for a period of time that ranges between any two of the preceding values.

In accordance with various embodiments, after the second culture operation, the process 1400 includes determining whether the cells require DO monitoring at operation 1440. In accordance with various embodiments, if the cells require DO monitoring, the approach 1400 includes a time-lapse imaging operation 1442. In accordance with various embodiments, the time-lapse imaging operation 1442 comprises the time-lapse imaging operation 1260 described herein with respect to FIG. 12.

In accordance with various embodiments, after operation 1440 (and after operation 1442, if relevant), the approach 1400 includes continuing the second culture operation 1430.

In accordance with various embodiments, after the second culture operation 1430, the approach 1400 includes repeating any of operations 1440, 1442, and 1430. In accordance with various embodiments, the approach 1400 includes repeating any of operations 1440, 1442, and 1430 at least about 1, 2, 3, 4, 5, 6, 7, 8, 9, 10, or more times. In accordance with various embodiments, the approach 1400 includes repeating any of operations 1440, 1442, and 14300 at most about 10, 9, 8, 7, 6, 5, 4, 3, 2, or 1 times. In accordance with various embodiments, the approach 1400 includes repeating any of operations 1440, 1442, and 1430 a number of times that ranges between any two of the preceding values.

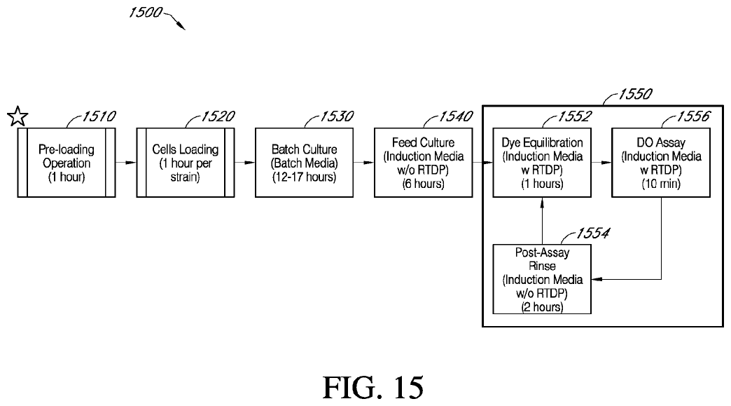

FIG. 15 illustrates a second example approach 1500 of performing a DO perfusion assay. In accordance with various embodiments, the approach 1500 includes a pre-loading operation 1510. In accordance with various embodiments, the pre-loading operation comprises wetting and optically calibrating a microfluidic device or chip, flushing the microfluidic device or chip with RTDP solution, equilibrating the microfluidic device or chip to an oxygen setpoint, and imaging the microfluidic device or chip to create a normalization reference image.

In accordance with various embodiments, the approach 1500 includes a cell loading operation 1520. In accordance with various embodiments, the cell loading operation comprises loading cells into sequestration pens of the microfluidic device or chip. The cell loading operation may be similar to the cell loading operation 1410 described herein with respect to FIG. 14.

In accordance with various embodiments, after the cell loading operation, the approach 1500 includes a batch culture operation 1530. In accordance with various embodiments, the batch culture operation comprises culturing the cells in the presence of BMGY growth medium, BMMY growth medium, Bird growth medium, Delft growth medium, LSM growth medium, or FM22 growth medium, or other liquid growth medium sufficient to support growth of cells. In accordance with various embodiments, the batch culture operation comprises culturing the cells for at least about 1 hour (h), 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, or more. In accordance with various embodiments, the batch culture operation comprises culturing the cells for at most about 24 h, 23 h, 22 h, 21 h, 20 h, 19 h, 18 h, 17 h, 16 h, 15 h, 14 h, 13 h, 12 h, 11 h, 10 h, 9 h, 8 h, 7 h, 6 h, 5 h, 4 h, 3 h, 2 h, 1 h, or less. In accordance with various embodiments, the batch culture operation comprises culturing the cells for a period of time that ranges between any two of the preceding values.

In accordance with various embodiments, after the batch culture operation, the approach 1500 includes a feed culture operation 1540. In accordance with various embodiments, the feed culture operation comprises culturing the cells in induction medium without RTDP. In accordance with various embodiments, the feed culture operation comprises culturing the cells for at least about 1 hour (h), 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, or more. In accordance with various embodiments, the feed culture operation comprises culturing the cells for at most about 24 h, 23 h, 22 h, 21 h, 20 h, 19 h, 18 h, 17 h, 16 h, 15 h, 14 h, 13 h, 12 h, 11 h, 10 h, 9 h, 8 h, 7 h, 6 h, 5 h, 4 h, 3 h, 2 h, 1 h, or less. In accordance with various embodiments, the feed culture operation comprises culturing the cells for a period of time that ranges between any two of the preceding values.

In accordance with various embodiments, after the feed culture operation, the approach 1500 includes an assay operation 1550. In accordance with various embodiments, the assay operation includes a dye equilibration operation 1552, a DO assay operation 1556, and a post-assay rinse operation 1554. In accordance with various embodiments, the dye equilibration operation comprises culturing the cells in induction medium with RTDP for a first period of time. In accordance with various embodiments, the first period of time is at least about 1 min, 2 min, 3 min, 4 min, 5 min, 6 min, 7 min, 8 min, 9 min, 10 min, 15 min, 20 min, 25 min, 30 min, 35 min, 40 min, 45 min, 50 min, 55 min, 1 h, 2 h, 3 h, 4 h, or more. In accordance with various embodiments, the first period of time is at most about 4 h, 3 h, 2 h, 1 h, 55 min, 50 min, 45 min, 40 min, 35 min, 30 min, 25 min, 20 min, 15 min, 10 min, 9 min, 8 min, 7 min, 6 min, 5 min, 4 min, 3 min, 2 min, 1 min, or less. In accordance with various embodiments, the first period of time ranges between any two of the preceding values. In accordance with various embodiments, the DO assay operation comprises culturing the cells in induction medium with RTDP for a second period of time, obtaining fluorescence images of the microfluidic device or chip, as described herein, and determining a DO level across the microfluidic device or chip from the fluorescence images, as described herein. In accordance with various embodiments, the second period of time is at least about 1 min, 2 min, 3 min, 4 min, 5 min, 6 min, 7 min, 8 min, 9 min, 10 min, 15 min, 20 min, 25 min, 30 min, 35 min, 40 min, 45 min, 50 min, 55 min, 1 h, 2 h, 3 h, 4 h, or more. In accordance with various embodiments, the second period of time is at most about 4 h, 3 h, 2 h, 1 h, 55 min, 50 min, 45 min, 40 min, 35 min, 30 min, 25 min, 20 min, 15 min, 10 min, 9 min, 8 min, 7 min, 6 min, 5 min, 4 min, 3 min, 2 min, 1 min, or less. In accordance with various embodiments, the second period of time ranges between any two of the preceding values. In accordance with various embodiments, the post-assay rinse operation comprises rinsing the cells for a third period of time. In accordance with various embodiments, the third period of time is at least about 1 min, 2 min, 3 min, 4 min, 5 min, 6 min, 7 min, 8 min, 9 min, 10 min, 15 min, 20 min, 25 min, 30 min, 35 min, 40 min, 45 min, 50 min, 55 min, 1 h, 2 h, 3 h, 4 h, or more. In accordance with various embodiments, the third period of time is at most about 4 h, 3 h, 2 h, 1 h, 55 min, 50 min, 45 min, 40 min, 35 min, 30 min, 25 min, 20 min, 15 min, 10 min, 9 min, 8 min, 7 min, 6 min, 5 min, 4 min, 3 min, 2 min, 1 min, or less. In accordance with various embodiments, the third period of time ranges between any two of the preceding values.

In accordance with various embodiments, the approach 1500 includes repeating any of operations 1550, 1552, 1554, and 1556. In accordance with various embodiments, the approach comprises repeating any of operations 1550, 1552, 1554, and 1556 at least about 1, 2, 3, 4, 5, 6, 7, 8, 9, 10, 11, 12, 13, 14, 15, 16, 17, 18, 19, 20, or more times. In accordance with various embodiments, the approach comprises repeating any of operations 1550, 1552, 1554, and 1556 at most about 20, 19, 18, 17, 16, 15, 14, 13, 12, 11, 10, 9, 8, 7, 6, 5, 4, 3, 2, or 1 times. In accordance with various embodiments, the approach comprises repeating any of operations 1550, 1552, 1554, and 1556 a number of times that ranges between any two of the preceding values.

### DO Edge Effect Mitigation

In accordance with various embodiments, the microfluidic device used to culture the biological micro-objects is permeable to gas flow. In accordance with various embodiments, oxygen may permeate from an environment surrounding the microfluidic device, or vice versa. In accordance with various embodiments, oxygen may permeate from one area of the microfluidic device to another area of the microfluidic device. In accordance with various embodiments, this “DO edge effect” may lead to non-uniform DO supply across the microfluidic device. In accordance with various embodiments, the DO edge effect may be mitigated using a variety of approaches.

In accordance with various embodiments, the DO edge effect is mitigated by coating exterior surfaces of the microfluidic device with an oxygen-impermeable film. In accordance with various embodiments, the microfluidic device comprises a plurality of exterior surfaces and at least a portion of one or more surfaces of the plurality are coated with an oxygen-impermeable film. In accordance with various embodiments, a portion of at least about 1, 2, 3, 4, 5, 6, or more exterior surfaces of the microfluidic device are coated with the oxygen-impermeable film. In accordance with various embodiments, a portion of at most about 6, 5, 4, 3, 2, or 1 exterior surfaces of the microfluidic device are coated with the oxygen-impermeable film. In accordance with various embodiments, a portion of a number of exterior surfaces of the microfluidic device that ranges between any two of the preceding values are coated with the oxygen-impermeable film. In accordance with various embodiments, the portion of the surface is at least about 10%, 20%, 30%, 40%, 50%, 60%, 70%, 80%, 90%, or more of the surface. In accordance with various embodiments, the portion of the surface is at most about 90%, 80%, 70%, 60%, 50%, 40%, 30%, 20%, 10%, or less of the surface. In accordance with various embodiments, the portion of the surface ranges between any two of the preceding values. In accordance with various embodiments, the portion of the surface comprises substantially all of the surface. In accordance with various embodiments, the portion of the one or more surfaces comprises those portions of the one or more surfaces which are permeable to oxygen if the oxygen-impermeable film is omitted.

In accordance with various embodiments, the oxygen-impermeable film has an oxygen permeability at 25° C. of at least about 1 cm³ mm·m⁻² day⁻¹atm⁻¹, 2 cm³ mm·m⁻² day⁻¹atm⁻¹, 3 cm³ mm·m⁻² day⁻¹atm⁻¹, 4 cm³ mm·m⁻² day⁻¹atm⁻¹, 5 cm³ mm·m⁻² day⁻¹atm⁻¹, 6 cm³ mm·m⁻² day⁻¹atm⁻¹, 7 cm³ mm·m⁻² day⁻¹atm⁻¹, 8 cm³ mm·m⁻² day⁻¹atm⁻¹, 9 cm³ mm·m⁻² day⁻¹atm⁻¹, 10 cm³ mm·m⁻² day⁻¹atm⁻¹, 20 cm³ mm·m⁻² day⁻¹atm⁻¹, or more. In accordance with various embodiments, the oxygen-impermeable film has an oxygen permeability at 25° C. of at most about 20 cm³ mm·m⁻² day⁻¹atm⁻¹, 10 cm³ mm·m⁻² day⁻¹atm⁻¹, 9 cm³ mm·m⁻² day⁻¹atm⁻¹, 8 cm³ mm·m⁻² day⁻¹atm⁻¹, 7 cm³ mm·m⁻² day⁻¹atm⁻¹, 6 cm³ mm·m⁻² day⁻¹atm⁻¹, 5 cm³mm·m⁻² day⁻¹atm⁻¹, 4 cm³ mm·m⁻² day⁻¹atm⁻¹, 3 cm³ mm·m⁻² day⁻¹atm⁻¹, 2 cm³ mm·m⁻² day⁻¹atm⁻¹, 1 cm³ mm·m⁻² day⁻¹atm⁻¹, or less. In accordance with various embodiments, the oxygen-impermeable film has an oxygen permeability at 25° C. that ranges between any two of the preceding values.

In accordance with various embodiments, the oxygen-impermeable film has a thickness of at least about 0.01 micrometers (μm), 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. In accordance with various embodiments, the oxygen-impermeable film has a thickness of at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm, 0.1 μm, or less. In accordance with various embodiments, the oxygen-impermeable film has a thickness that ranges between any two of the preceding values.

In accordance with various embodiments, the oxygen-impermeable film comprises any suitable material that can be applied to the one or more exterior surfaces of the microfluidic device and that reduces oxygen transfer from outside the microfluidic device to inside the microfluidic device. In accordance with various embodiments, the oxygen-impermeable film comprises Parylene N (poly(p-xylene)), Parylene C (poly(2-chloro-1,4-dimethylbenzene), Parylene D (poly(2,5-dichloro-1,4-dimethylbenzene)), Parylene HT (poly(1,4-Bis(difluoromethyl)benzene)), epoxy, Torr-Seal epoxy, or any combination thereof. In accordance with various embodiments, the oxygen-impermeable film is applied to a microfluidic device (such as microfluidic device 200 described herein with respect to FIG. 2A) using chemical vapor deposition, a conformal coating process, physical application of the film material such as by brushing, spraying, dipping, or dispensing from an applicator, or any other application process that is effective to provide the oxygen-impermeable film on the microfluidic device. In accordance with various embodiments, portions of the exterior surfaces of the microfluidic device are masked off with a masking material before the oxygen-impermeable film is applied to the microfluidic device, which is followed by removal of the masking material to provide microfluidic device having portions of the exterior surfaces that are not covered by the oxygen-impermeable film and portions of the exterior surfaces that are covered by the oxygen-impermeable film. In various embodiments, the portions of the exterior surfaces that are not covered by the oxygen-impermeable film can include portions of the microfluidic device that provide a functional interface to the microfluidic device, such as a port, inlet, outlet, electrical contact, or optical interface (for example, an optically transparent cover, or the like, for imaging chambers or flow regions or areas of interest (AOIs) or for projecting structured light onto a surface of the device to activate DEP forces within a DEP substrate), or can include portions of the microfluidic device that are constructed of materials which are oxygen impermeable. In various embodiments, the portions of the exterior surfaces that are covered by the oxygen-impermeable film can include portions of the microfluidic device that are constructed of materials that are not oxygen-impermeable or are oxygen permeable, or can include portions of the exterior surfaces of the microfluidic device that, in the absence of the oxygen-impermeable film, would permit diffusion of oxygen from the exterior of the device into a flow region or chamber of the microfluidic device, or can include other portions of the exterior surfaces.

In accordance with various embodiments, the DO edge effect is mitigated using an oxygen delivery system (such as oxygen delivery system 1600 described herein with respect to FIG. 16) to deliver a supplied partial pressure of oxygen to at least a portion of one or more exterior surfaces of the plurality. In accordance with various embodiments, the oxygen delivery module is configured to couple to any microfluidic device described herein (such as microfluidic device 200 described herein with respect to FIG. 2A).

In accordance with various embodiments, the DO edge effect is mitigated by delivering a supplied partial pressure of oxygen to at least a portion of one or more exterior surfaces of the plurality. In accordance with various embodiments, the supplied partial pressure of oxygen is at least about 0.01 bar, 0.02 bar, 0.03 bar, 0.04 bar, 0.05 bar, 0.06 bar, 0.07 bar, 0.08 bar, 0.09 bar, 0.1 bar, 0.11 bar, 0.12 bar, 0.13 bar, 0.14 bar, 0.15 bar, 0.16 bar, 0.17 bar, 0.18 bar, 0.19 bar, 0.2 bar, 0.21 bar, 0.22 bar, 0.23 bar, 0.24 bar, 0.25 bar, 0.3 bar, 0.4 bar, 0.5 bar, 0.6 bar, 0.7 bar, 0.8 bar, 0.9 bar, 1 bar, or more. In accordance with various embodiments, the supplied partial pressure of oxygen is at most about 1 bar, 0.9 bar, 0.8 bar, 0.7 bar, 0.6 bar, 0.5 bar, 0.4 bar, 0.3 bar, 0.25 bar, 0.24 bar, 0.23 bar, 0.22 bar, 0.21 bar, 0.2 bar, 0.19 bar, 0.18 bar, 0.17 bar, 0.16 bar, 0.15 bar, 0.14 bar, 0.13 bar, 0.12 bar, 0.11 bar, 0.1 bar, 0.09 bar, 0.08 bar, 0.07 bar, 0.06 bar, 0.05 bar, 0.04 bar, 0.03 bar, 0.02 bar, 0.01 bar, or less. In accordance with various embodiments, the supplied partial pressure of oxygen ranges between any two of the preceding values.

In accordance with various embodiments, the supplied partial pressure of oxygen is delivered to at least the portion of the one or more exterior surfaces by one or more tubes placed in proximity to the one or more exterior surface. FIG. 16 shows an oxygen delivery system 1600 comprising one or more tubes 1602 with one or more holes (lumens) 1604. The one or more tubes are configured to be connected to a gas source for receiving a gaseous medium comprising the supplied partial pressure of oxygen. In accordance with various embodiments, the one or more tubes comprise one or more holes configured to allow the supplied partial pressure of oxygen to flow therethrough. In accordance with various embodiments, the one or more tubes comprise at least about 1, 2, 3, 4, 5, 6, 7, 8, 9, 10, 20, 30, 40, 50, 60, 70, 80, 90, 100, or more holes. In accordance with various embodiments, the one or more tubes comprise at most about 100, 90, 80, 70, 60, 50, 40, 30, 20, 10, 9, 8, 7, 6, 5, 4, 3, 2, or 1 holes. In accordance with various embodiments, the one or more tubes comprise a range of tubes defined by any two of the preceding values.

In accordance with various embodiments, the DO edge effect is mitigated by surrounding the microfluidic device in an oxygen bath. In accordance with various embodiments, the supplied partial pressure of oxygen is delivered to the at least the portion of the one or more exterior surfaces by an oxygen bath surrounding the microfluidic device. In accordance with various embodiments, the supplied partial pressure of oxygen is at least about 0.01 bar, 0.02 bar, 0.03 bar, 0.04 bar, 0.05 bar, 0.06 bar, 0.07 bar, 0.08 bar, 0.09 bar, 0.1 bar, 0.11 bar, 0.12 bar, 0.13 bar, 0.14 bar, 0.15 bar, 0.16 bar, 0.17 bar, 0.18 bar, 0.19 bar, 0.2 bar, 0.21 bar, 0.22 bar, 0.23 bar, 0.24 bar, 0.25 bar, 0.3 bar, 0.4 bar, 0.5 bar, 0.6 bar, 0.7 bar, 0.8 bar, 0.9 bar, 1 bar, or more. In accordance with various embodiments, the supplied partial pressure of oxygen is at most about 1 bar, 0.9 bar, 0.8 bar, 0.7 bar, 0.6 bar, 0.5 bar, 0.4 bar, 0.3 bar, 0.25 bar, 0.24 bar, 0.23 bar, 0.22 bar, 0.21 bar, 0.2 bar, 0.19 bar, 0.18 bar, 0.17 bar, 0.16 bar, 0.15 bar, 0.14 bar, 0.13 bar, 0.12 bar, 0.11 bar, 0.1 bar, 0.09 bar, 0.08 bar, 0.07 bar, 0.06 bar, 0.05 bar, 0.04 bar, 0.03 bar, 0.02 bar, 0.01 bar, or less. In accordance with various embodiments, the supplied partial pressure of oxygen ranges between any two of the preceding values.

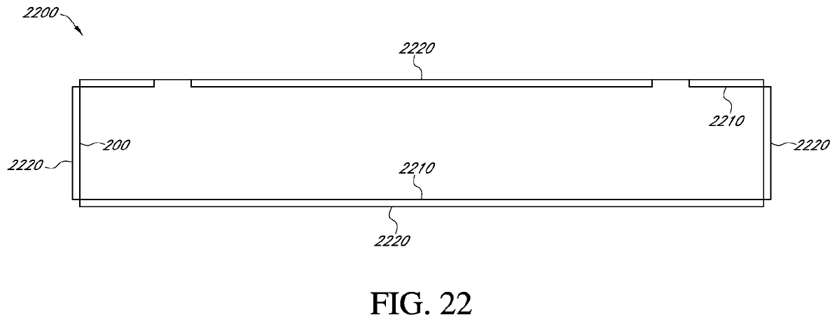

FIG. 22 shows a system 2200 configured to implement the methods described herein. In accordance with various embodiments, the system comprises a microfluidic device 200. In accordance with various embodiments, the microfluidic device is similar to any microfluidic device described herein, such as microfluidic device 200 described herein with respect to FIG. 2A. In accordance with various embodiments, the microfluidic device comprises a plurality of exterior surfaces 2210. In accordance with various embodiments, at least a portion of one or more exterior surfaces of the plurality are coated with an oxygen-impermeable film 2220. In accordance with various embodiments, the oxygen-permeable film is similar to any oxygen-impermeable film described herein.

In accordance with various embodiments, the oxygen-impermeable film has an oxygen permeability of at most 20 cm³ mm·m⁻² day⁻¹atm⁻¹. In accordance with various embodiments, the oxygen-impermeable film comprises Parylene N, Parylene C, Parylene D, Parylene HT, epoxy, Torr-Seal epoxy, or any combination thereof. In accordance with various embodiments, the oxygen-impermeable film has a thickness of at least 1 nanometer (nm). In accordance with various embodiments, the oxygen-impermeable film has a thickness of at most 10 micrometers (μm).

In accordance with various embodiments, the system further comprises an oxygen delivery module (not shown in FIG. 22) configured to deliver a supplied partial pressure of oxygen to at least a portion of one or more exterior surfaces of the plurality. In accordance with various embodiments, the oxygen delivery module comprises one or more tubes placed in proximity to the one or more exterior surfaces, the one or more tubes comprising one or more holes (lumens) configured to allow the supplied partial pressure of oxygen to flow therethrough. In accordance with various embodiments, the one or more tubes are similar to system 1600 described herein with respect to FIG. 16. In accordance with various embodiments, the oxygen delivery module comprises an oxygen bath surrounding the microfluidic device.

Further disclosed herein is a first kit configured to implement the methods described herein. In accordance with various embodiments, the kit comprises: any microfluidic device described herein (such as microfluidic device 200 described herein with respect to FIG. 2A, or such as the microfluidic device wherein at least a portion of one or more exterior surfaces are coated with an oxygen-impermeable film as described herein with respect to FIG. 22) and a buffer. In accordance with various embodiments, the kit further comprises a fluidic medium containing a dye. In accordance with various embodiments, the dye comprises a soluble and diffusible dye. In accordance with various embodiments, the dye comprises a ruthenium complex. In accordance with various embodiments, the dye comprises any dye described herein.

Further disclosed herein is a second kit configured to implement the methods described herein. In accordance with various embodiments, the kit comprises: any microfluidic device described herein (such as microfluidic device 200 described herein with respect to FIG. 2A, or such as the microfluidic device wherein at least a portion of one or more exterior surfaces are coated with an oxygen-impermeable film as described herein with respect to FIG. 22); and a fluidic medium containing a dye. In accordance with various embodiments, the dye comprises a soluble and diffusible dye. In accordance with various embodiments, the dye comprises a ruthenium complex. In accordance with various embodiments, the dye comprises any dye described herein. In accordance with various embodiments, the kit further comprises a buffer.

### EXPERIMENTAL

### Example 1. Assay for Detection of a Level of Dissolved Oxygen in a Fluid Located within a Microfluidic Device

System and Microfluidic device: The foregoing experiments were performed using an OptoSelect™ microfluidic (or nanofluidic) device manufactured by Berkeley Lights, Inc. and controlled by an optical instrument which was also manufactured by Berkeley Lights, Inc. The instrument included: a mounting stage for the microfluidic device coupled to a temperature controller; a pump and fluid medium conditioning component; an optical train including a camera and a structured light source suitable for activating phototransistors within the microfluidic device; and software for controlling the instrument, including performing image analysis and automated detection and repositioning of micro-objects. The OptoSelect™ device included a substrate configured with OptoElectroPositioning (OEP™) technology, which provides a phototransistor-activated dielectrophoresis (DEP) force. The device also included a plurality of microfluidic channels, each having a plurality of NanoPen™ chambers (or sequestration pens) fluidically connected thereto. The volume of each sequestration pen was around 1×10⁶ cubic microns. The microfluidic device included conditioned interior surfaces, which are described in U.S. Patent Application Publication No. US2016/0312165 (Lowe, Jr., et al.), International Patent Application Publication WO2017/205830 (Lowe, Jr., et al.), and International Patent Application Publication WO2019/01880 (Beemiller et al.), each of which disclosures is herein incorporated by reference in its entirety.

Priming regime: 250 microliters of 100% carbon dioxide was flowed in at a rate of 12 microliters/sec. This was followed by 250 microliters of a wetting solution including surface conditioning reagents to provide conditioned surfaces as described in the referenced publications above.

Culture medium: a minimal phosphate buffered aqueous medium was used in each of Examples 1-5.

Cells: Yeast of the Saccharomycetaceae family were used in each of Examples 1-5.

Reference Curve for Dissolved Oxygen (DO) Assay: A reference curve was generated for use in the DO assay for image normalization. A primed microfluidic device as described above, was used, without any cells present within the sequestration pens. An initial calibration image was acquired using the 4X objective, before inputting any fluidic medium, and used to correct raw images obtained throughout the experiment.

The reference curve was generated using at least five different oxygen concentration levels, ranging from 21% O₂, e.g., Clean Dry Air (CDA) to 2.02% O₂, using custom pre-mixed gas (Praxair). The five concentrations used were: CDA, 14.14%, 7.98%, 5.19%, and 2.02%. In this experiment, a sixth gas, N₂ (e.g., 0% O₂), was also used. Generally, however, the five-point reference curve from 21% O₂ to 2.02% O₂ provided sufficiently reproducible and representative reference curves for the Dissolved Oxygen assay. In some variations, a reference curve may be obtained using four, three or two O₂ reference points and still provide robust detection of dissolved oxygen levels. In other variations, a reference curve may be obtained using at least six, seven, eight, nine, ten, or more, reference points.

For each O₂ concentration, the following steps were performed. Culture medium containing RTDP (2 mg/ml; Aldrich Cat. No. 544981-1G. Tris(2,2′-bipyridyl)-dichlororuthenium(II) hexahydrate; (Ru(BPY)3) was sparged with the specific gas mixture prior to introduction into the system. The microfluidic device was flushed with the sparged culture medium at 1 microliter/sec for 250 sec. The microfluidic device was then flushed with the specific gas mixture for 60 min at 5 microliters/sec. Sparged culture medium with RTDP (2 mg/ml) was perfused at 3 microliters/sec for at least 20 min, preferably 30 min. After this period of equilibration, the flow rate was increased to 4 microliters/sec. The cycle of perfusion provides periods of perfusion punctuated by intervals of no flow. Images were obtained at one minute intervals during both portions of the perfusion cycle over a period of at least 5 min, with excitation at 455 nm. Images centered on an Area of Interest (AOI) centered mid-pen within the sequestration pens as well as a set of images centered within the channel were obtained at 625 nm with 50 msec illumination (15% power) using a custom bandpass filter combination. While this experiment uses a mid-pen location for the AOI, a useful AOI for the reference curve generation and for the DO assay may be any region within the sequestration pen where medium transference is dominated by diffusion. The values obtained were normalized against the data obtained from the 21% O₂ (CDA) images. The average normalized intensity across the microfluidic device in the channel region for each O₂ concentration was plotted and is shown in FIG. 9A (flow) and FIG. 9B (no flow). The respective average normalized intensity across all of the sequestration pens were plotted against each O₂ concentration and is shown in FIG. 9C (flow) and FIG. 9D (no flow). What can be noted is that the normalized values of O₂ concentration within the mid-pen assay areas are very similar, despite the different conditions of flow or no flow in the channel, implying that the mid-pen region is a stable AOI.

In variations of this reference curve experiment, a liquid flush sparged with the respective concentration of O₂ replaced the gas flush performed at each O₂ concentration, prior to introduction of the sparged RTDP-containing culture medium present during imaging. Additionally, the use of N₂ gas as a value for zero O₂ concentration was eliminated. The lowest concentration of O₂ included generally in reference curve generation was 2.02%.

Dissolved Oxygen Assay. Cells of interest were then loaded into the sequestration pens of the microfluidic device and cultured 15 h at 30° C. with a sparged culture medium (CDA gas) after an initial ten minute period perfusing a CDA gas/CDA-sparged culture medium mixture (80:20 v/v) at 0.1 microliter/sec. After this initial period, the CDA gas/CDA-sparged culture medium mixture (80:20 v/v) perfusion also included RTDP at a concentration of 2 mg/ml, at 1 microliter/sec at 27° C. followed, for a 5 h period.

At the end of the 5 hr culture period, the sequestration pens were examined to identify each sequestration pen having no cells within the AOI. The optical density (OD) of each identified sequestration pen was obtained under brightfield, and quantified by comparison to the OD of that sequestration pen when empty under brightfield.

Fluorescence images at 625 nm were obtained across the microfluidic device, in the channel region and within each identified sequestration pen at the mid-pen AOI, either at a single timepoint or over a ten minute period, using 50 msec illumination, 15% power as above. For each identified sequestration pen, e.g., a pen having no cells within the mid-pen AOI, the raw fluorescence values were averaged if more than one image per AOI was taken. The raw fluorescence was normalized against the fluorescence observed in that pen prior to cell importation at 21% O₂ concentration to remove pen to pen aberrations. The normalized (optionally averaged) fluorescence value was finally correlated to the dissolved O₂ level from the reference curve generated above, which can be represented as O₂ saturation percentage. When each correlated dissolved O₂ level of the respective sequestration pen was plotted against the OD obtained for that pen (correlating with Biomass of the sequestration pen), the relationship was observed as shown in FIG. 8, where values of biomass along the x-axis are binned for easier review. The relationship is roughly linear, but there was clonal variation observable. For a given biomass point, a range of dissolved oxygen concentrations for a set of sequestration pens is shown in FIG. 8, extending in the y-space. Colonies in the sequestration pens having a higher concentration of O₂ (greater amount of O₂ saturation) are consuming less oxygen than the sequestration pens having an equivalent biomass (OD) where lower concentration of O₂ was imaged, e.g., more fluorescence signal). The more fluorescent, less O₂ saturated, faster O₂ consuming colonies can be selected for further analysis, as the sequestration pen identification was maintained throughout the assay. The cells from preferred sequestration pens may be either the colonies having the highest biomass or may be pens where the cells have the highest O₂ consumption per mass unit where the colony may not have the most number of cells but the individual cells are consuming oxygen at the highest rate per cell.

### Example 2. DO Standard Curve

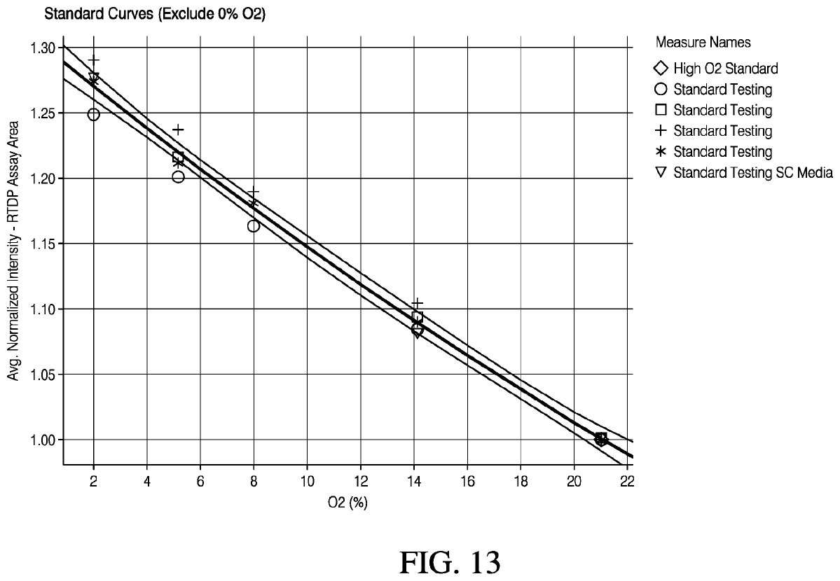

DO standard curve. FIG. 13 illustrates exemplary DO standard curves generated by the process 1200. As shown in FIG. 13, the dynamic range included air saturation values from 9.5% air saturation to 100% air saturation. The normalized RTDP was converted to a DO value and achieved a coefficient of variation (CV) of 1.4%.

### Example 3. RTDP Concentration Testing

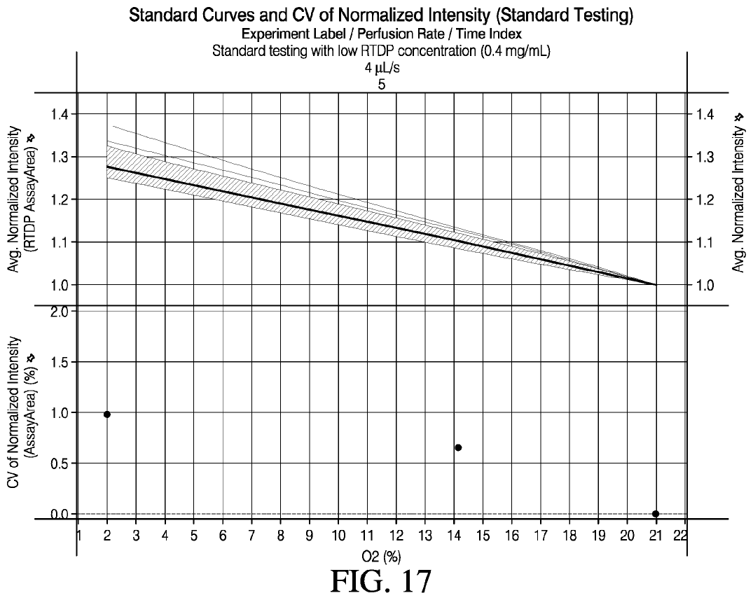

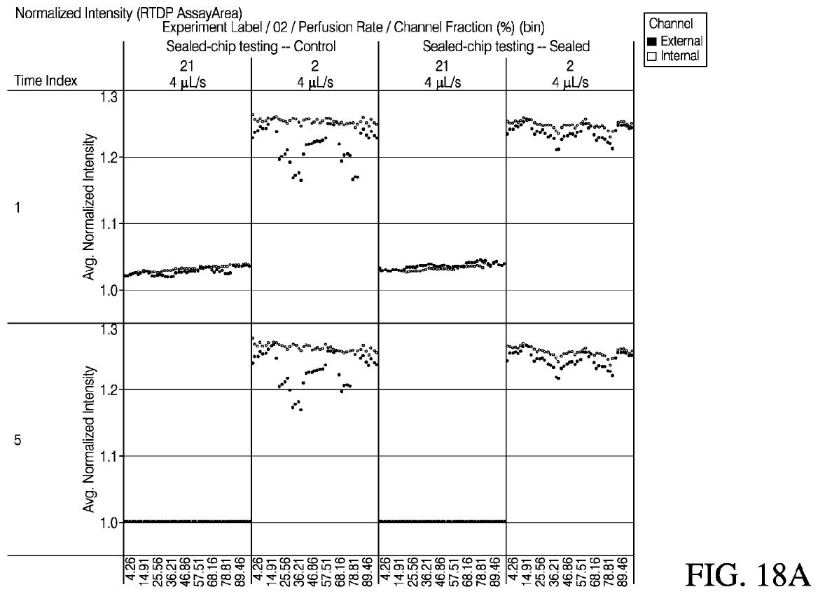

RTDP concentration testing. The RTDP concentration was decreased from a standard value of 2 mg/mL to determine whether RTDP concentration affected DO assay performance. A RTDP concentration of 0.4 mg/mL was prepared and testing was conducted at nominal O₂ concentrations of 21%, 14%, and 2%. The fluorescence exposure was set to 150 ms with 15% illumination. FIG. 17 shows the variability of the normalized fluorescence intensity at a 0.4 mg/mL RTDP concentration. As shown in FIG. 17, the variability of the normalized intensity for the 0.4 mg/mL RTDP concentration was within tolerable limits and within the same range as a 2 mg/mL RTDP concentration.

### Example 4. Torr-Seal Epoxy Sealing of Microfluidic Devices

Torr-seal chip sealing. Mitigation of the DO edge effect was tested using Torr-Seal epoxy sealing, as described herein. The results of an unsealed microfluidic device and a microfluidic device sealed using Torr-Seal epoxy were compared. The RTDP concentration was 0.4 mg/mL and a fluorescence exposure of 150 ms with 15% illumination was utilized. The nominal O₂ concentration was varied between 21% and 2%. The sparging and gas flush time was varied between 15 minutes and 60 minutes. A first perfusion of RTDP was performed at 3 μL/s and the time of the first perfusion was varied between 5 minutes and 25 minutes. A second perfusion of RTDP was performed at 4 μL/s and the time of the second perfusion was 5 minutes.

### Example 5: Parylene Sealing of Microfluidic Devices

Parylene chip sealing. Mitigation of the DO edge effect was tested using Parylene sealing, as described herein. The results of an unsealed microfluidic device surrounded by an O₂ supply and a microfluidic device sealed using Parylene were compared. The RTDP concentration was 0.4 mg/mL and a fluorescence exposure of 150 ms with 15% illumination was utilized. The nominal O₂ concentration was varied between 21% and 2%. The sparging and gas flush time was varied between 15 minutes and 60 minutes. A first perfusion of RTDP was performed at 3 μL/s and the time of the first perfusion was varied between 15 minutes and 25 minutes. A second perfusion of RTDP was performed at 4 μL/s and the time of the second perfusion was 10 minutes.

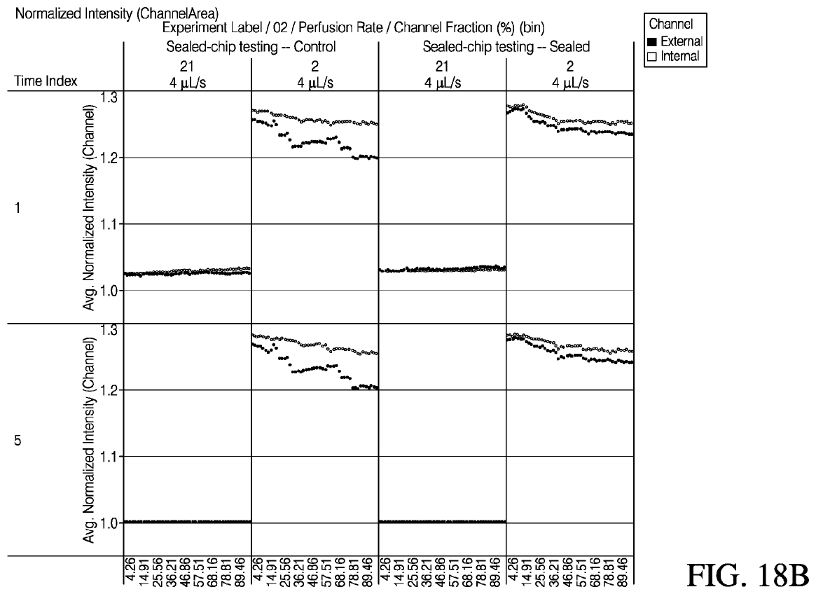

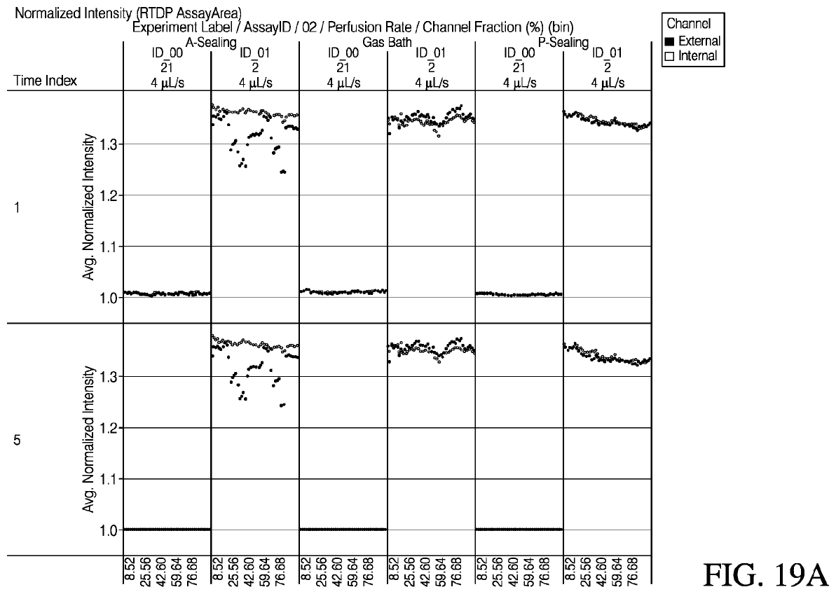

FIGS. 18A-18B shows the improvement in dissolved oxygen uniformity achieved by sealing the microfluidic chip from external gas exchange using Parylene. The x-axis shows distance along the channel (from inlet to outlet) expressed as a 100% of the total length of the channel from inlet to outlet. The y-axis is an average normalized intensity of fluorescence signal that correlates with dissolved oxygen level. Decreased signal correlates with increased oxygen. Along the top axis is the uniformity of signal for 21% oxygen (ambient air) and 2% oxygen in both control chips (no sealing) and Parylene-sealed chips, with FIGS. 18A and 18B being the measurement in the pen (AssayArea) and channel (ChannelArea), respectively. At the 21% condition, there was relatively uniform signal across the channel length for all conditions since the oxygen level inside the chip was pumped (at 4 μL/s as noted in the top axis) to match the ambient level. However, when 2% was utilized within the chip, there was more severe non-uniformities in the unsealed chip as the signal in the external channels (dark circle) decreases to reflect gas diffusing into the channels closest to the edge. The Parylene sealing reduces this and brings external and internal channel signal together. The greatest nonuniformity was observed right after turns where the external channels were closest to the edge of the chip. This was true over time as shown in the two different time indexes 1 and 5 (after 1 minute and after 5 minutes, respectively).

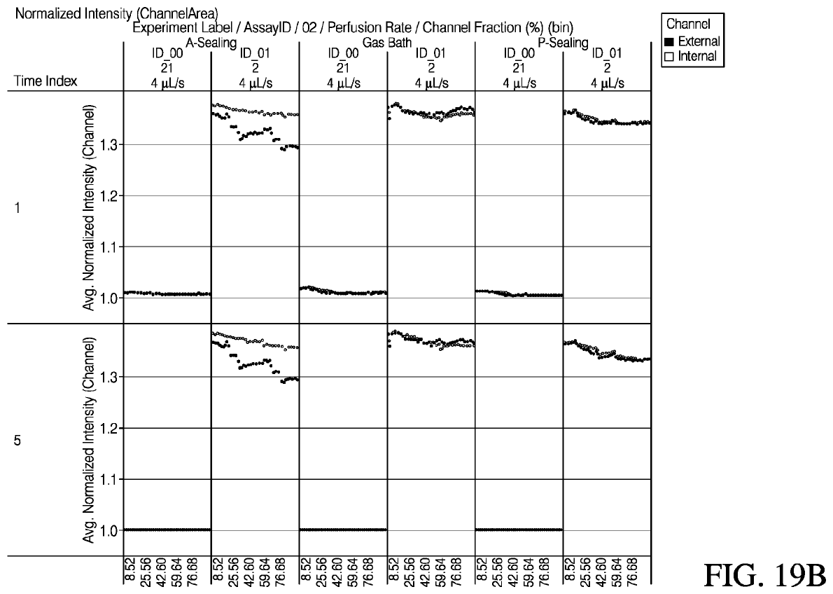

FIGS. 19A-19B shows the different performance levels of various sealing techniques in limiting external gas exchange to improve dissolved oxygen uniformity. The x-axis shows distance along the channel (from inlet to outlet) expressed as a 100% of the total length of the channel from inlet to outlet. The y-axis is an average normalized intensity of fluorescence signal that correlates with dissolved oxygen level. Decreased signal correlates with increased oxygen. Along the top axis is the uniformity of signal for 21% oxygen (ambient air) and 2% oxygen in 3 different sealing conditions: A-sealed (sealed using Torr-Seal), gas bath, and P-sealed (sealed using Parylene), with FIGS. 19A and 19B being the measurement in the pen (AssayArea) and channel (Channel Area), respectively. At the 21% condition, there was relatively uniform signal across the channel length for all conditions since the oxygen level inside the chip was pumped (at 4 μL/s as noted in the top axis) to match the ambient level. However, when 2% was utilized within the chip, there non-uniformities in the external channels (dark circle) of several conditions. Non-uniform signal (drop in signal associated with increased oxygen as it diffuses through the outside wall of the chip) was most severe in A-sealed chips, somewhat present with the gas bath, and not present with the P-sealed chip. Again, note that the greatest nonuniformity was observed right after turns when the external channels are closest to the edge of the chip. This was true over time as we see in the two different time indexes 1 and 5 (after 1 minute and after 5 minutes, respectively).

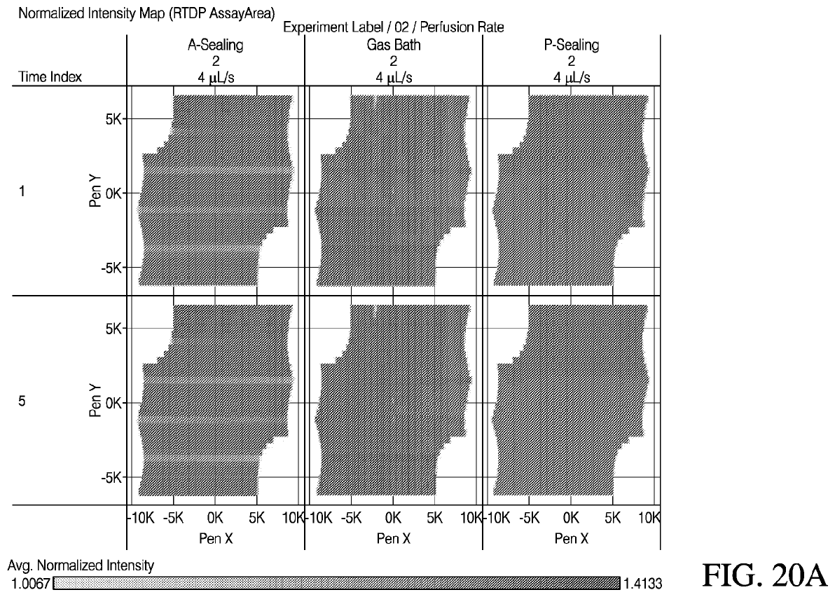

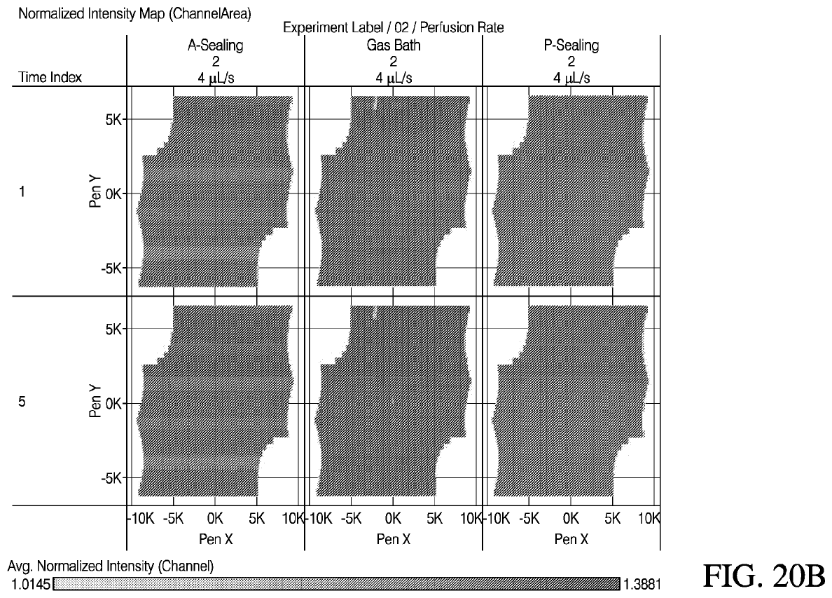

FIGS. 20A-20B show an example of how the above-described non-uniformities in external gas exchange impact the dissolved oxygen signal as observed over the whole chip. The lower legends provide an intensity map from white to black, where white is the lowest average normalized intensity. As described above, decreased signal correlates with increased oxygen (i.e. the whiter bands are gas exchange with the external channels). Along the top axis is the uniformity of signal across the chip for 2% oxygen (at 4 μL/s) in 3 different sealing conditions: A-sealed, gas bath, and P-sealed, with FIGS. 20A and 20B being the measurement in the pen (AssayArea) and channel (Channel Area), respectively. As shown in the quantification above, A-Sealed chips have stark white bands associated with the channels that face the external surface when turning, pick up oxygen and decrease the assay signal. Gas-bath non-uniformities were less severe and absent from the P-sealed chip.

### Example 6: Pen-Level DO Measurements

Pen-level DO measurements. Growth medium with oxygen-sensitive dye RTDP was flushed through the chip at a high flow rate while fluorescence images were captured. The constant flow allowed the channel area to act as an oxygen source. Live cell colonies in the pen bottoms consumed oxygen at a rate determined by the number of cells and the cells' biological characteristics, thereby acting as an oxygen sink. The difference in oxygen consumption between sink and source creates a steady-state fluorescence gradient in the oxygen-sensitive dye between the cell colony and the top of the pen meeting the channel.

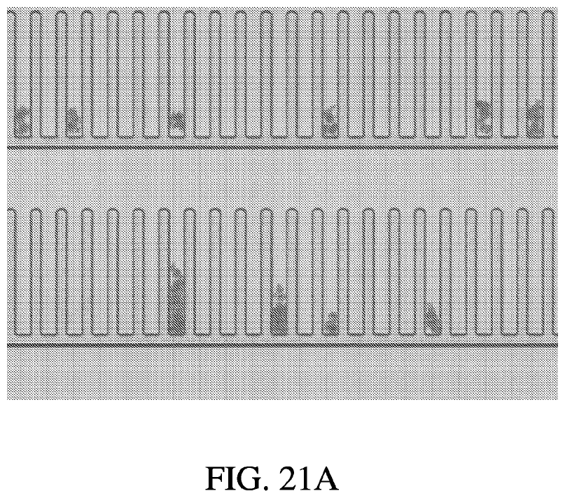

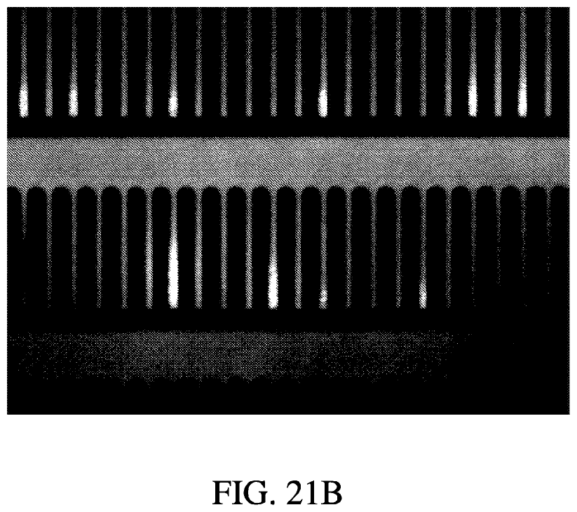

FIG. 21A shows an exemplary brightfield image of cells in sequestration pens. The boundary of the cell colony was determined by automated image analysis of the brightfield image. The fluorescence intensity of the RTDP signal in the DO consumption assay was quantified in a region of interest dynamically defined by the boundary of the cell colony, allowing quantification of the differential fluorescence between the cell colony and the top of the pen. The magnitude of this differential fluorescence signal was normalized against the spatial variation of illumination intensity in the brightfield image, the size of the cell colony, and the autofluorescence signal from the colony, giving a measure of the characteristic oxygen consumption of the strain in that pen.

The RTDP was dissolved in the same media that was used for the induction culture period of the workflow described herein. Because the DO assay can be repeated, the cells were alternatingly cultured in the regular induction media and the RTDP media for the assay. These cells had been culturing for approximately 30 hours total on chip since they were loaded.

FIG. 21B shows an exemplary fluorescence image of dissolved oxygen in the sequestration pens. As shown in FIG. 21B, sequestration pens containing cells produced fluorescence signals in proportion to the number of cells contained therein, while sequestration pens containing no cells did not produce fluorescence signals.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical, FLASH memory and non-optical data storage devices. The computer readable medium can also be distributed over a network coupled computer systems so that the computer readable code is stored and executed in a distributed fashion.

### Listing of Embodiments

Embodiment 1. A method of determining a level of oxygen in a medium disposed within a microfluidic device comprising a flow region and one or more chambers fluidically coupled to the flowing region, the method comprising:
flowing a fluidic medium containing a dye and a supplied partial pressure of oxygen into the microfluidic device for a period of time, wherein fluorescence emitted by the dye changes based on availability of oxygen proximate to the dye;
taking a fluorescence image of an area of interest (AOI) within the flow region or one or more of the chambers; and
correlating fluorescence detected in the fluorescence image of the AOI with a reference to determine an observed level (e.g., a partial pressure) of oxygen in the AOI.

Embodiment 2. The method of embodiment 1, further comprising:
determining a level of oxygen consumption by a biological micro-object or a population of biological micro-objects (e.g., a clonal population) disposed within one of the one or more chambers.

Embodiment 3. The method of embodiment 2, further comprising:
comparing the determined level of oxygen consumption with a threshold value; and
selecting the biological micro-object or the population of biological micro-objects (e.g., a clonal population) if the determined level of oxygen consumption is above the threshold value.

Embodiment 4. The method of embodiment 2 or 3, further comprising:
forecasting a level of productivity of an expanded population of biological micro-objects expanded from the biological micro-object or the population of biological micro-objects based at least in part upon the determined level of oxygen consumption.

Embodiment 5. The method of embodiment 4, further comprising:
determining a number of biological micro-objects present in the chamber, wherein the forecast level of productivity is based at least in part on the determined number of biological micro-objects in the chamber.

Embodiment 6. The method of embodiment 4 or 5, further comprising:
comparing the forecast level of productivity with a threshold value; and
selecting the biological micro-object or the population of biological micro-objects (e.g., a clonal population) if the forecast level of productivity is above the threshold value.

Embodiment 7. The method of embodiment 3 or 6, wherein the selected biological micro-object or the population of biological micro-objects is removed from the microfluidic device (e.g., exported) and, optionally, cultured so as to produce an expanded population of biological micro-objects.

Embodiment 8. The method of embodiment 7, wherein the expanded population of biological micro-objects is expanded at least partially following export from the microfluidic device (e.g., in a macro-scale culture device, which can be any culture device having a volume that can be used for cell culture of at least 1 mL).

Embodiment 9. A method of determining a level of oxygen consumption by a biological micro-object or a population of biological micro-objects, the method comprising:
optionally disposing the biological micro-object or the clonal population of biological micro-objects in a chamber of a microfluidic device comprising a flow region, wherein the chamber is fluidically connected to the flow region;
flowing a fluidic medium containing a dye and a supplied partial pressure of oxygen into the microfluidic device for a period of time, wherein fluorescence emitted by the dye changes based on availability of oxygen proximate to the dye;
taking a fluorescence image of an area of interest within the flow region and/or the chamber;
correlating fluorescence detected in the fluorescence image of the area of interest with a reference to determine an observed level (e.g., partial pressure) of oxygen in the area of interest; and
determining the level of oxygen consumption by the biological micro-object or the population of biological micro-objects disposed within the chamber.

Embodiment 10. A method of selecting a biological micro-object or a population of biological micro-objects, the method comprising:
optionally disposing the biological micro-object or the clonal population of biological micro-objects in a chamber of a microfluidic device comprising a flow region, wherein the chamber is fluidically connected to the flow region;
flowing a fluidic medium containing a dye and a supplied partial pressure of oxygen into the microfluidic device for a period of time, wherein fluorescence emitted by the dye changes based on availability of oxygen proximate to the dye;
taking a fluorescence image of an area of interest within the flow region and/or the chamber;
correlating fluorescence detected in the fluorescence image of the area of interest with a reference to determine an observed level (e.g., partial pressure) of oxygen in the area of interest;
determining the level of oxygen consumption by the biological micro-object or the population of biological micro-objects disposed within the chamber; and
selecting the biological micro-object or the population of biological micro-objects if the determined level of oxygen consumption is above a threshold value.

Embodiment 11. A method of forecasting a level of productivity of a population of biological micro-objects expanded from a biological micro-object or a clonal population of biological micro-objects, the method comprising:
optionally disposing a biological micro-object or a clonal population of biological micro-objects in a chamber of a microfluidic device comprising a flow region, wherein the chamber is fluidically connected to the flow region;
flowing a fluidic medium containing a dye and a supplied partial pressure of oxygen into the microfluidic device for a period of time, wherein fluorescence emitted by the dye changes based on availability of oxygen proximate to the dye;
taking a fluorescence image of an area of interest within the flow region and/or the chamber;
correlating fluorescence detected in the fluorescence image of the area of interest with a reference to determine an observed level (e.g., partial pressure) of oxygen in the area of interest;
determining a level of oxygen consumption by the biological micro-object or the population of biological micro-objects disposed within the chamber; and
forecasting a level of productivity of the expanded population of biological micro-objects expanded from the biological micro-object or the clonal population of biological micro-objects, wherein the forecast level of productivity is based at least in part upon the determined level of oxygen consumption.

Embodiment 12. The method of embodiment 10 or 11, wherein the microfluidic device comprises a plurality of chambers, each fluidically connected to the flow region, wherein there is a plurality of biological micro-objects and/or populations of biological micro-objects, each one of the biological micro-objects and/or populations of biological micro-objects disposed within a corresponding chamber of the plurality of chambers, and wherein selecting the biological micro-object or the population of biological micro-objects comprises selecting one or more of the plurality of biological micro-objects and/or populations of biological micro-objects.

Embodiment 13. The method of any one of embodiments 1-12, wherein the dye comprises a soluble and diffusible dye.

Embodiment 14. The method of any one of embodiments 1-13, wherein the dye comprises a ruthenium complex.

Embodiment 15. The method of any one of embodiments 1-14, wherein the fluorescence emitted by the dye is quenched when the dye is in proximity to oxygen and fluoresces when the dye is not in proximity to oxygen.

Embodiment 16. The method of any one of embodiments 1-15, wherein the level of oxygen consumption corresponds to a decrease in the observed partial pressure of the oxygen compared to the supplied partial pressure.

Embodiment 17. The method of any one of embodiments 1-16, wherein the fluidic medium is flowed at a flow rate ranging between 0.1 microliter/s and 10 microliters/s.

Embodiment 18. The method of any one of embodiments 1-17, wherein the biological micro-object or population of biological micro-objects in the chamber of the microfluidic device is cultured under aerobic conditions comprising flowing a fluidic medium through the fluidic region, wherein the fluidic medium comprises at least a minimum supplied partial pressure of oxygen of 0.04 bar.

Embodiment 19. The method of any one of embodiments 1-18, wherein the AOI is disposed within the chamber at a location wherein transference of components of the fluidic medium flowing in the flow region (e.g., a channel to which the chamber is fluidically connected) is dominated by diffusion.

Embodiment 20. The method of any one of embodiments 1-19, wherein the AOI is disposed in the flow region (e.g., a channel), at a position proximal to an opening from the chamber to the flow region.

Embodiment 21. The method of any one of embodiments 1-20, wherein the AOI contains no biological micro-objects.

Embodiment 22. The method of any one of embodiments 1-21, wherein the fluidic medium comprises a liquid medium, a gaseous medium, or a mixture thereof.

Embodiment 23. The method of any one of embodiments 1-22, wherein the flowing the fluidic medium containing the dye and the supplied partial pressure of oxygen into the microfluidic device comprises alternately flowing a liquid medium into the microfluidic device and flowing a gaseous medium comprising the supplied partial pressure of oxygen into the microfluidic device.

Embodiment 24. The method of any one of embodiments 1-23, wherein the medium comprises a liquid medium saturated with the supplied partial pressure of the oxygen.

Embodiment 25. The method of any one of embodiments 1-24, wherein the correlating the fluorescence of the fluorescence image of the AOI to a reference to determine the observed partial pressure of the oxygen at the AOI comprises identifying an intensity of the fluorescence on a reference curve correlating fluorescence intensities of the dye with supplied partial pressures of the oxygen.

Embodiment 26. The method of any one of embodiments 1-25, wherein the method further comprises constructing a reference curve correlating an observed intensity of fluorescence of the dye with a supplied partial pressure of oxygen.

Embodiment 27. The method of embodiment 26, wherein the constructing the reference curve comprises:
flowing a liquid medium through the channel of the microfluidic device for a first reference period of time, wherein the liquid medium comprises the dye and a first supplied partial pressure of the oxygen;
detecting a first fluorescence intensity of the dye within a selected region of the microfluidic device;
flowing a second liquid medium through the channel of the microfluidic device for a second reference period of time, wherein the second liquid medium comprises the dye and a second supplied partial pressure of the oxygen;
detecting a second fluorescence intensity of the dye within the selected region of the microfluidic device; and
correlating each of the first and the second fluorescence intensities with the first and second supplied partial pressures of the oxygen, respectively.

Embodiment 28. The method of embodiment 26, wherein the constructing the reference curve further comprises flowing liquid media comprising the dye and successive supplied partial pressures of the oxygen at successive points in time; detecting successive fluorescence intensities of the dye at the successive points in time; and correlating each of the fluorescence intensities with a supplied partial pressure of the oxygen.

Embodiment 29. The method of any one of embodiments 26-28, wherein the microfluidic device does not contain any biological micro-objects while constructing the reference curve.

Embodiment 30. The method of any one of embodiments 26-29, wherein the selected supplied partial pressure of oxygen is from about 0.02 bar to about 0.21 bar.

Embodiment 31. The method of any one of embodiments 26-30, wherein the method further comprises detecting fluorescence intensities associated with at least three, four, five, or more supplied partial pressures of the oxygen.

Embodiment 32. The method of any one of embodiments 1-31, wherein the fluorescence image is taken under a perfusion condition.

Embodiment 33. The method of embodiment 32, wherein the perfusion condition comprises a constant flow, a steady state flow, a pre-programmed flow with one or more defined flow rates, or zero flow after equilibrium.

Embodiment 34. The method of any one of embodiments 1-33, wherein the microfluidic device comprises a plurality of chambers, and wherein the method further comprises:
introducing the population of biological micro-objects into the plurality of chambers.

Embodiment 35. The method of embodiment 34, wherein the flow region of the microfluidic device comprises a plurality of channels, and wherein the method further comprises:
introducing the population of biological micro-objects into the plurality of channels.

Embodiment 36. The method of any one of embodiments 1-35, wherein the flowing the fluidic medium and the taking the fluorescence image are performed at a selected temperature.

Embodiment 37. The method of embodiment 36, wherein the temperature is from about 20° C. to about 40° C.

Embodiment 38. The method of embodiment 36 or 37, wherein the temperature is from about 28° C. to about 30° C.

Embodiment 39. The method of any one of embodiments 1-38, wherein the flowing the fluidic medium and the taking the fluorescence image is performed at a selected pH.

Embodiment 40. The method of embodiment 39, wherein the pH is from about 3.0 to about 9.0.

Embodiment 41. The method of any one of embodiments 1-40, wherein the method further comprises taking a plurality of fluorescence images at a plurality of timestamps and correlating a respective fluorescence of each fluorescence image to determine a respective observed partial pressure of the oxygen in the AOI at the respective timestamp.

Embodiment 42. The method of any one of embodiments 1-41, wherein the method further comprises taking a plurality of fluorescence images at a plurality of points within the AOI.

Embodiment 43. The method of any one of embodiments 1-42, wherein the chamber comprises a sequestration pen, wherein the sequestration comprises an isolation region and a connection region, and wherein the connection region has a proximal opening to the channel and a distal opening to the isolation region.

Embodiment 44. The method of embodiment 43, wherein the isolation region comprises a single opening to the connection region.

Embodiment 45. The method of embodiment 43 or 44, wherein the population of biological micro-objects is disposed within the isolation region of the sequestration pen.

Embodiment 46. The method of any one of embodiments 43-45, wherein the method further comprises taking a plurality of fluorescence images at a plurality of points extending from the isolation region of the sequestration pen along an axis of diffusion towards the channel.

Embodiment 47. The method of any one of embodiments 43-46, wherein the AOI comprises at least part of the connection region.

Embodiment 48. The method of any one of embodiments 1-47, wherein the microfluidic device comprises a plurality of exterior surfaces, wherein each of the plurality of exterior surface is oxygen-impermeable.

Embodiment 49. The method of any one of embodiments 1-48, wherein the microfluidic device comprises a plurality of exterior surfaces and wherein at least a portion of one or more exterior surfaces of the plurality is coated with an oxygen-impermeable film.

Embodiment 50. The method of embodiment 49, wherein the oxygen-impermeable film has an oxygen permeability of 20 cm³ mm·m⁻² day⁻¹atm⁻¹ or less.

Embodiment 51. The method of embodiment 49 or 50, wherein the oxygen-impermeable film has an oxygen permeability of between 1 cm³ mm·m⁻² day⁻¹atm⁻¹ and 20 cm³ mm·m⁻² day⁻¹atm⁻¹.

Embodiment 52. The method of any one of embodiments 49-51, wherein the oxygen-impermeable film comprises Parylene N, Parylene C, Parylene D, Parylene HT, epoxy, Torr-Seal epoxy, or any combination thereof.

Embodiment 53. The method of any one of embodiments 49-52, wherein the oxygen-impermeable film has a thickness of at least 1 nanometer (nm).

Embodiment 54. The method of any one of embodiments 49-53, wherein the oxygen-impermeable film has a thickness of at most 10 micrometers (μm).

Embodiment 55. The method of any one of embodiments 1-54, wherein the microfluidic device comprises a plurality of exterior surfaces and wherein the method further comprises delivering a supplied partial pressure of oxygen to at least a portion of one or more exterior surfaces of the plurality.

Embodiment 56. The method of embodiment 55, wherein the supplied partial pressure of oxygen is delivered to at least the portion of the one or more exterior surfaces by one or more tubes placed in proximity to the one or more exterior surfaces, the one or more tubes comprising one or more holes (or lumens) configured to allow the supplied partial pressure of oxygen to flow therethrough.

Embodiment 57. The method of embodiment 55 or embodiment 56, wherein the supplied partial pressure of oxygen is delivered to the at least the portion of the one or more exterior surfaces by an oxygen bath surrounding the microfluidic device.

Embodiment 58. A system comprising:
a microfluidic device comprising:
a flow region (e.g. comprising a channel);
a chamber configured to receive a population of biological micro-objects therein, wherein the chamber opens to the flow region (or channel); and
a plurality of exterior surfaces, wherein each exterior surface of the plurality is impermeable to oxygen.

Embodiment 59. A system comprising:
a microfluidic device comprising:
a flow region (e.g., comprising a channel);
a chamber configured to receive a population of biological micro-objects therein, wherein the chamber opens to the flow region; and
a plurality of exterior surfaces, wherein at least a portion of one or more exterior surfaces of the plurality are coated with an oxygen-impermeable film.

Embodiment 60. The system of embodiment 59, wherein the oxygen-impermeable film has an oxygen permeability of at least 1 cm³ mm·m⁻² day⁻¹atm⁻¹.

Embodiment 61. The system of embodiment 59 or 60, wherein the oxygen-impermeable film has an oxygen permeability of at most 20 cm³ mm·m⁻² day⁻¹atm⁻¹.

Embodiment 62. The system of any one of embodiments 59-61, wherein the oxygen-impermeable film comprises Parylene N, Parylene C, Parylene D, Parylene HT, epoxy, Torr-Seal epoxy, or any combination thereof.

Embodiment 63. The system of any one of embodiments 59-62, wherein the oxygen-impermeable film has a thickness of at least 1 nanometer (nm).

Embodiment 64. The system of any one of embodiments 59-63, wherein the oxygen-impermeable film has a thickness of at most 10 micrometers (μm).

Embodiment 65. The system of any one of embodiments 59-64, wherein the microfluidic device comprises a plurality of chambers, each chamber of the plurality configured to receive a population of biological micro-objects therein.

Embodiment 66. The system of any one of embodiments 59-65, wherein the flow region of the microfluidic device comprises a plurality of channels.

Embodiment 67. The system of any one of embodiments 59-66, wherein the chamber comprises a sequestration pen, wherein the sequestration pen comprises an isolation region and a connection region, and wherein the connection region has a proximal opening to the flow region (or a channel of the flow region) and a distal opening to the isolation region.

Embodiment 68. The system of embodiment 67, wherein the isolation region comprises a single opening to the connection region.

Embodiment 69. The system of embodiment 67 or 68, wherein the isolation region of the sequestration pen is configured to receive the population of biological micro-objects therein.

Embodiment 70. A system comprising:
an oxygen delivery module;
a nest comprising a support structure configured to support a microfluidic device in proximity to the oxygen delivery module;
a gas source in fluidic communication with the oxygen delivery module; and
a controller configured to control a flow of gas from the gas source to the oxygen delivery module.

Embodiment 71. The system of embodiment 70, wherein the oxygen delivery module comprises one or more tubes, the one or more tubes comprising one or more holes configured to allow a supplied partial pressure of oxygen to flow therethrough.

Embodiment 72. The system of embodiment 70 or 71, wherein the oxygen delivery module comprises an oxygen bath surrounding the microfluidic device.

Embodiment 73. The system of any one of embodiments 70-72, wherein the nest is configured to provide a fluidic connection between the system and said microfluidic device.

Embodiment 74. The system of any one of embodiments 70-73, wherein the nest further comprises a socket configured to provide an electrical interface between the system and said microfluidic device.

Embodiment 75. The system of any one of embodiments 70-74, further comprising a fluidic medium source comprising a sparging component in fluidic communication with the gas source.

Embodiment 76. The system of any one of embodiments 70-75, wherein the system further comprises a structured light source positioned to direct structured light to said microfluidic device and configured to thereby activate phototransistors within said microfluidic device.

Embodiment 77. The system of any one of embodiments 70-76, further comprising a microfluidic device disposed on the support structure, the microfluidic device comprising.
a flow region (e.g., comprising a channel);
a chamber configured to receive a population of biological micro-objects therein, wherein the chamber opens to the flow region.

Embodiment 78. The system of any one of embodiments 70-77, wherein the microfluidic device comprises a plurality of chambers, each chamber of the plurality configured to receive a population of biological micro-objects therein.

Embodiment 79. The system of any one of embodiments 70-78, wherein the flow region of the microfluidic device comprises a plurality of channels.

Embodiment 80. The system of any one of embodiments 70-79, wherein the chamber comprises a sequestration pen, wherein the sequestration comprises an isolation region and a connection region, and wherein the connection region has a proximal opening to the flow region (or a channel of the flow region) and a distal opening to the isolation region.

Embodiment 81. The system of embodiment 80, wherein the isolation region comprises a single opening to the connection region.

Embodiment 82. A kit comprising:
a microfluidic device comprising:
a flow region (e.g., comprising a channel);
a chamber configured to receive a population of biological micro-objects therein, wherein the chamber opens to the flow region (or a channel of the flow region); and
a plurality of exterior surfaces, wherein each exterior surface of the plurality is impermeable to oxygen or wherein at least a portion of one or more exterior surfaces of the plurality are coated with an oxygen-impermeable film; and
a buffer.

Embodiment 83. The kit of embodiment 82, further comprising a fluidic medium containing a dye configured to emit a fluorescence signal that changes based on availability of oxygen proximate to the dye.

Embodiment 84. The kit of embodiment 83, wherein the dye comprises a soluble and diffusible dye.

Embodiment 85. The kit of embodiment 83 or 84, wherein the dye comprises a ruthenium complex.

Embodiment 86. A kit comprising:
a microfluidic device comprising:
a flow region (e.g., comprising a channel);
a chamber configured to receive a population of biological micro-objects therein, wherein the chamber opens to the flow region (or a channel of the flow region); and
a plurality of exterior surfaces, wherein each exterior surface of the plurality is impermeable to oxygen or wherein at least a portion of one or more exterior surfaces of the plurality are coated with an oxygen-impermeable film; and
a fluidic medium containing a dye configured to emit a fluorescence signal that changes based on availability of oxygen proximate to the dye.

Embodiment 87. The kit of embodiment 86, wherein the dye comprises a soluble and diffusible dye.

Embodiment 88. The kit of embodiment 86 or 87, wherein the dye comprises a ruthenium complex.

Embodiment 89. The kit of any one of embodiments 86-88, further comprising a buffer.

1. A method of determining a level of oxygen in a medium disposed within a microfluidic device comprising a flow region and one or more chambers fluidically coupled to the flowing region, the method comprising:
flowing a fluidic medium containing a dye and a supplied partial pressure of oxygen into the microfluidic device for a period of time, wherein fluorescence emitted by the dye changes based on availability of oxygen proximate to the dye;
taking a fluorescence image of an area of interest (AOI) within the flow region or one or more of the chambers; and
correlating fluorescence detected in the fluorescence image of the area of interest with a reference to determine an observed level of oxygen in the area of interest.

2. The method of claim 1, further comprising:
determining a level of oxygen consumption by a biological micro-object or a population of biological micro-objects disposed within one of the one or more chambers.

3. The method of claim 2, further comprising:
comparing the determined level of oxygen consumption with a threshold value; and
selecting the biological micro-object or the population of biological micro-objects if the determined level of oxygen consumption is above the threshold value.

4. The method of claim 2, further comprising:
forecasting a level of productivity of an expanded population of biological micro-objects expanded from the biological micro-object or the population of biological micro-objects based at least in part upon the determined level of oxygen consumption.

5. The method of claim 4, further comprising:
determining a number of biological micro-objects present in the chamber, wherein the forecast level of productivity is based at least in part on the determined number of biological micro-objects in the chamber.

6. The method of claim 4, further comprising:
comparing the forecast level of productivity with a threshold value; and
selecting the biological micro-object or the population of biological micro-objects if the forecast level of productivity is above the threshold value.

7. (canceled)

8. (canceled)

9. (canceled)

10. (canceled)

11. The method of claim 1, wherein the dye comprises a ruthenium complex.

12. The method of claim 1, wherein the area of interest is disposed within the chamber at a location wherein transference of components of the fluidic medium flowing in the flow region is dominated by diffusion.

13. The method of claim 1, wherein the flowing the fluidic medium containing the dye and the supplied partial pressure of oxygen into the microfluidic device comprises alternately flowing a liquid medium into the microfluidic device and flowing a gaseous medium comprising the supplied partial pressure of oxygen into the microfluidic device.

14. (canceled)

15. (canceled)

16. (canceled)

17. (canceled)

18. (canceled)

19. The method of claim 1, wherein the method further comprises taking a plurality of fluorescence images at a plurality of timestamps and correlating a respective fluorescence of each fluorescence image to determine a respective observed partial pressure of the oxygen in the area of interest at the respective timestamp.

20. (canceled)

21. (canceled)

22. (canceled)

23. (canceled)

24. (canceled)

25. The method of claim 1, wherein the microfluidic device comprises a plurality of exterior surfaces and wherein at least a portion of one or more exterior surfaces of the plurality is coated with an oxygen-impermeable film.

26. The method of claim 25, wherein the oxygen-impermeable film has an oxygen permeability of between 1 cm³ mm·m⁻² day⁻¹atm⁻¹ and 20 cm³ mm·m⁻² day⁻¹atm⁻¹.

27. (canceled)

28. (canceled)

29. (canceled)

30. (canceled)

31. A system comprising:
a microfluidic device comprising:
a flow region;
a chamber configured to receive a population of biological micro-objects therein, wherein the chamber opens to the flow region; and
a plurality of exterior surfaces, wherein at least a portion of one or more exterior surfaces of the plurality are coated with an oxygen-impermeable film.

32. The system of claim 31, wherein the oxygen-impermeable film has an oxygen permeability of at most 20 cm³ mm·m⁻² day⁻¹atm⁻¹.

33. The system of claim 31, wherein the oxygen-impermeable film has a thickness of at least 1 nanometer (nm).

34. (canceled)

35. The system of claim 31, wherein the microfluidic device comprises a plurality of chambers, each chamber of the plurality configured to receive a population of biological micro-objects therein.

36. (canceled)

37. A system comprising:
an oxygen delivery module;
a nest comprising a support structure configured to support a microfluidic device in proximity to the oxygen delivery module;
a gas source in fluidic communication with the oxygen delivery module; and
a controller configured to control a flow of gas from the gas source to the oxygen delivery module.

38. The system of claim 37, wherein the oxygen delivery module comprises one or more tubes, the one or more tubes comprising one or more holes configured to allow a supplied partial pressure of oxygen to flow therethrough.

39. The system of claim 37, wherein the oxygen delivery module comprises an oxygen bath surrounding the microfluidic device.

40. The system of claim 37, wherein the nest: is configured to provide a fluidic connection between the system and said microfluidic device; and/or further comprises a socket configured to provide an electrical interface between the system and said microfluidic device.

41. (canceled)

42. The system of claim 37, further comprising a fluidic medium source comprising a sparging component in fluidic communication with the gas source.

43. The system of claim 37, wherein the system further comprises a structured light source positioned to direct structured light to said microfluidic device and configured to thereby activate phototransistors within said microfluidic device.

44. (canceled)

45. (canceled)

46. (canceled)

47. (canceled)

48. (canceled)

49. (canceled)

50. (canceled)
